(19) United States
(12) Patent Application Publication
(10) Pub. No.: US 2024/0024578 A1
(43) Pub. Date: Jan. 25, 2024

(54) AMBULATORY MEDICAMENT PUMP WITH DISTRESS NOTIFICATIONS

(71) Applicant: Beta Bionics, Inc., Irvine, CA (US)

(72) Inventors: Himanshu Patel, Rancho Santa Margarita, CA (US); Michael J. Rosinko, Las Vegas, NV (US); Firasd H. El-Khatib, Allston, MA (US); Edward R. Damiano, Acton, MA (US)

(21) Appl. No.: 18/354,009

(22) Filed: Jul. 18, 2023

Related U.S. Application Data
(60) Provisional application No. 63/368,748, filed on Jul. 18, 2022.

(51) Int. Cl. A61M 5/172

(52) U.S. Cl. CPC A61M 5/1723; A61M 2205/18; A61M 2230/201; A61M 2005/14208

(57) ABSTRACT

A glucose level control system may be configured to determine that certain status information fails to satisfy at least one of a plurality of safety thresholds. The system may receive status information, which may include device information pertaining to a condition of the ambulatory medicament device and/or subject information pertaining to a condition of a subject. The system can determine that the status information fails to satisfy the safety threshold(s). In response to determining that the status information fails to satisfy the at least one of a plurality of safety thresholds, the system can automatically execute a distress action. Example safety thresholds include a glucose level of a subject being less than a minimum threshold, a glucose level of a subject being greater than a maximum threshold, a movement of the subject being less than a minimum movement threshold for a threshold amount of time, and/or other safety thresholds.

### BACKGROUND

### Technical Field

This disclosure relates to glucose control systems, including medical devices that provide glucose control therapy to a subject, glucose level control systems, and ambulatory medicament pumps that deliver medicament to the subject to control blood glucose level in the subject.

### Description of Related Art

Sustained delivery, pump driven medicament injection devices generally include a delivery cannula mounted in a subcutaneous manner through the skin of the subject at an infusion site. The pump draws medicine from a reservoir and delivers it to the subject via the cannula. The injection device typically includes a channel that transmits a medicament from an inlet port to the delivery cannula which results in delivery to the subcutaneous tissue layer where the delivery cannula terminates. The delivery of medicament by the pump is controlled by a controller based on values of one or more control parameters and/or a measured glucose level in the subject. Some infusion devices are configured to deliver one medicament to a subject while others are configured to deliver multiple medicaments to a subject.

### BRIEF DESCRIPTION OF THE DRAWINGS

To easily identify the discussion of any particular element or act, the most significant digit or digits in a reference number refer to the figure number in which that element is first introduced.

FIG. 1A illustrates an example blood glucose control system that provides blood glucose control via an ambulatory medicament pump.

FIG. 1B illustrates another example blood glucose control system that provides blood glucose control via an ambulatory medicament pump.

FIG. 1C illustrates a further example blood glucose control system that provides blood glucose control via an ambulatory medicament pump.

FIG. 2A shows a block diagram of an example blood glucose control system.

FIG. 2B shows a block diagram of another example blood glucose control system.

FIG. 2C shows a block diagram of another example blood glucose control system.

FIG. 2D shows a block diagram of another example blood glucose control system.

FIG. 3 is a schematic of an example glucose control system that includes an electronic communications interface.

FIG. 4A shows a block diagram of an example blood glucose control system in online operation mode.

FIG. 4B shows a block diagram of an example blood glucose control system in offline operation mode.

FIG. 5A illustrates a perspective view of an example ambulatory medical device.

FIG. 5B illustrates a cross sectional view of the ambulatory medical device shown in FIG. 5A.

FIG. 6 illustrates different systems that may be included in an example ambulatory medical pump (AMP).

FIG. 7 illustrates various communication links that the AMP may use to establish a communication connection with an electronic device.

FIG. 8 illustrates the interconnection among modules and procedures in AMD involved in receiving, accepting and/or canceling therapy change request.

FIG. 9 is a flow diagram illustrating an example method that may be used by an AMD to generate an alarm status indicator.

FIG. 10 is a schematic diagram illustrating the interconnection among modules and procedures in AMD involved in monitoring the status of the AMD and/or the subject and generate alarms when an alarm condition is met.

FIG. 11 illustrates a block diagram of a glucose level control system in accordance with certain embodiments.

FIG. 12 illustrates a block diagram of a controller system in accordance with certain embodiments.

FIG. 13 presents a flowchart of an example carbohydrate therapy equivalence tracking process in accordance with certain embodiments.

FIG. 14 illustrates an example backup therapy protocol in accordance with certain embodiments.

FIG. 15 illustrates an example meal selection report that may be included as part of some implementations of the control parameter modification report of FIG. 11 in accordance with certain embodiments.

FIG. 16 presents a flowchart of an example automated glucose control refinement process in accordance with certain embodiments.

FIG. 17A illustrates a simulation of glucose control of a subject with Tmax set to 65 minutes.

FIG. 17B illustrates a simulation of glucose control of a subject with Tmax set to 15 minutes.

FIG. 17C illustrates a simulation of glucose control of a subject with Tmax set to 130 minutes.

FIG. 18 illustrates an example of blood glucose level signal (CGM trace) and some of the parameters associated with glycemic control using a glucose level control system.

FIG. 19 shows a flow diagram illustrating an example method that may be used by a control system to receive status information via a monitoring system interface, according to one embodiment.

FIG. 20 shows a flow diagram illustrating an example method that may be used by a control system for reviewing device operation history of an ambulatory medicament device, according to one embodiment.

FIG. 21 shows an example graph of a subject's blood glucose level (in mg/dL) over a 24-hour period.

FIG. 22 is a flow diagram illustrating an example procedure that may be used by the alert system of an AMD to monitor the operation of an AMD and generate alerts when a device malfunction is detected.

FIG. 23 illustrates an aspect of the subject matter in accordance with one embodiment.

FIG. 24 is a schematic diagram illustrating the interconnection among modules and procedures of an AMD involved in monitoring the status of the AMD and/or the subject and generating alarms when an alarm condition is met.

FIG. 25 is a flow diagram illustrating an example procedure that may be used by the alert system of an AMD to monitor the operation of an AMD and generate alerts when a device malfunction is detected.

FIG. 26 shows a flow diagram illustrating an example method that may be used by a control system to determine that the ambulatory medicament pump fails to meet a manufacturer specification and to transmit a request for a replacement ambulatory medicament pump or other ambulatory medicament device.

FIG. 27 shows a flow diagram illustrating an example method that may be used by a control system to determine that a use state of an ambulatory medicament device satisfies at least one of a plurality of user interaction criteria and to automatically generate a user alert comprising a link to training content.

FIG. 28 shows a flow diagram illustrating an example method that may be used by a control system to transmit a request for a remote support system to contact a user.

FIG. 29 shows a flow diagram illustrating an example method that may be used by a control system to determine that status information fails to satisfy at least one of a plurality of safety thresholds and to automatically generate a distress alert.

### DETAILED DESCRIPTION

### Blood Glucose Control System Overview

A blood glucose control system (BGCS) is used to control blood glucose level in a subject. Blood glucose control systems can include a controller configured to generate dose control signals for one or more glucose control agents that can be infused into the subject. Glucose control agents include regulatory agents that tend to decrease blood glucose level, such as insulin and insulin analogs, and counter-regulatory agents that tend to increase blood glucose level, such as glucagon or dextrose. A blood glucose control system configured to be used with two or more glucose control agents can calculate a dose control signal for each of the agents. In some embodiments, a blood glucose control system can calculate a dose control signal for an agent even though the agent may not be available for dosing via a medicament pump connected to the subject.

Glucose control agents can be delivered to a subject via subcutaneous injection, via intravenous injection, or via another suitable delivery method. In the case of blood glucose control therapy via an ambulatory medicament pump, subcutaneous injection is most common. An ambulatory medicament pump 100 is a type of ambulatory medical device, which is sometimes referred to herein as an ambulatory device, an ambulatory medicament device, a mobile ambulatory device, or an AMD. Ambulatory medical devices include ambulatory medicament pumps and other devices configured to be carried by a subject and to deliver therapy to the subject.

In some examples, the ambulatory medical device (AMD) is an electrical stimulation device, and therapy delivery includes providing electrical stimulation to a subject. An example of an electrical stimulation device is a cardiac pacemaker. A cardiac pacemaker generates electrical stimulation of the cardiac muscle to control heart rhythms. Another example of an electrical stimulation device is a deep brain stimulator to treat Parkinson's disease or movement disorders.

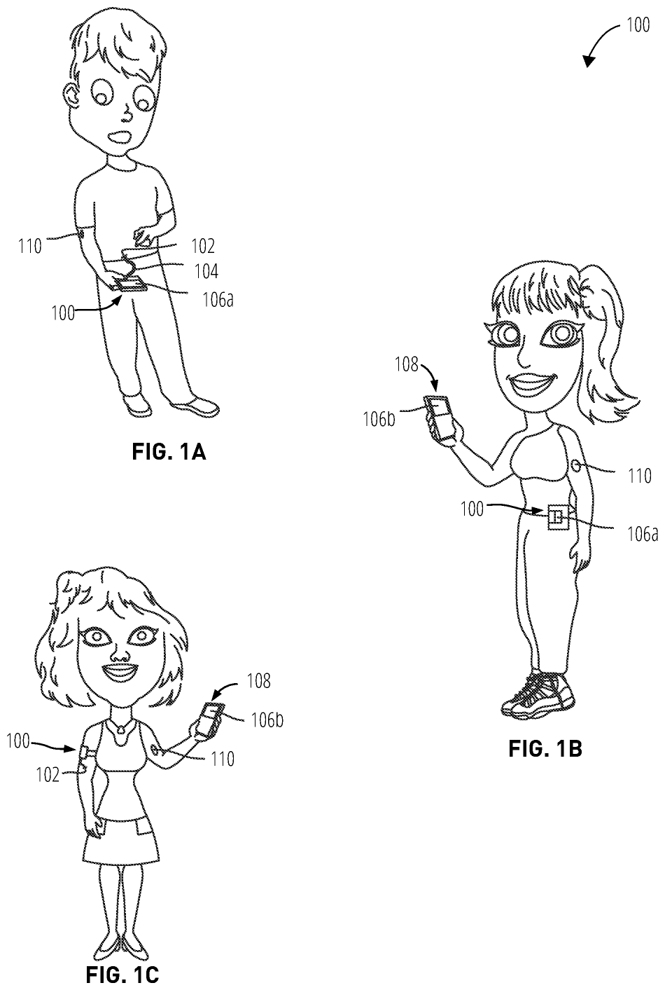

FIG. 1A-FIG. 1C show examples of blood glucose control systems that provide blood glucose control via an ambulatory medical device or AMD, such as a medicament pump connected to a subject. In FIG. 1A, the AMD 100 (medicament pump) is connected to an infusion site 102 using an infusion set 104. The AMD 100 (medicament pump) has integrated pump controls 106a that permit a user to view pump data and change therapy settings via user interaction with the pump controls 106a. A glucose level sensor 110 generates a glucose level signal that is received by the blood glucose control system.

In FIG. 1B, the medicament pump 100 communicates with an external electronic device 108 (such as, for example, a smartphone) via a wireless data connection. At least some of the pump controls 106a and 106b can be manipulated via user interaction with user interface elements of the external electronic device 108. The glucose level sensor 110 can also communicate with the AMD 100 (medicament pump) via a wireless data connection.

In FIG. 1C, the AMD 100 (medicament pump) includes an integrated cannula that inserts into the infusion site 102 without a separate infusion set. At least some of the pump controls 106b can be manipulated via user interaction with user interface elements of an external electronic device 108. In some instances, pump controls can be manipulated via user interaction with user interface elements generated by a remote computing environment (not shown), such as, for example, a cloud computing service, that connects to the AMD 100 (medicament pump) via a direct or indirect electronic data connection.

Glucose control systems typically include a user interface configured to provide one or more of therapy information, glucose level information, and/or therapy control elements capable of changing therapy settings via user interaction with interface controls. The user interface can be implemented via an electronic device that includes a display and one or more buttons, switches, dials, capacitive touch interfaces, or touchscreen interfaces. In some embodiments, at least a portion of the user interface is integrated with an ambulatory medicament pump that can be tethered to a body of a subject via an infusion set configured to facilitate subcutaneous injection of one or more glucose control agents. In certain embodiments, at least a portion of the user interface is implemented via an electronic device separate from the ambulatory medicament pump, such as a smartphone.

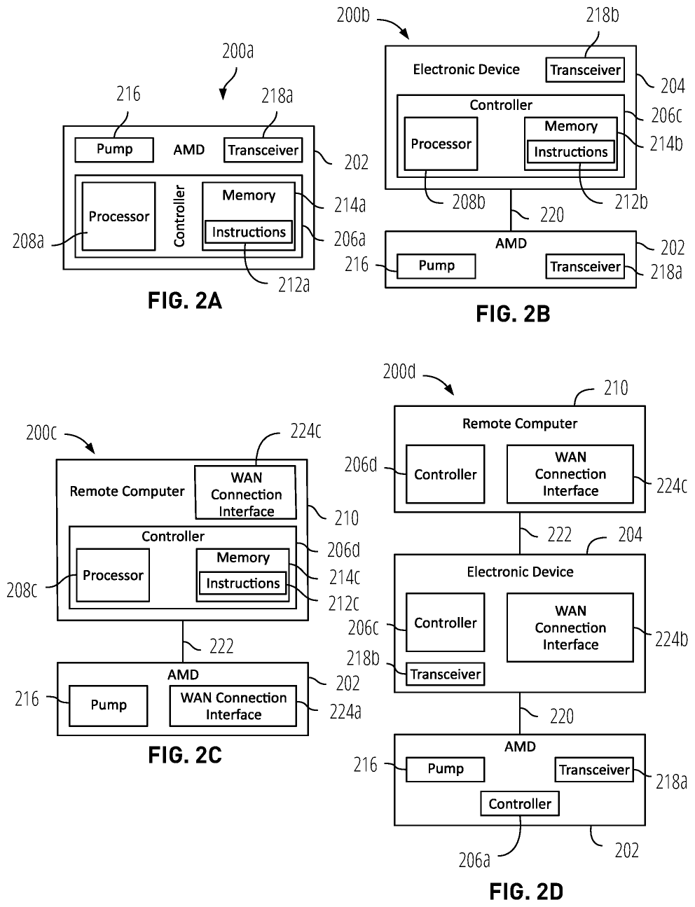

FIG. 2A-FIG. 2D illustrate block diagrams showing example configurations of a glucose control system 200a/200b/200c/200d. As shown in FIG. 2A, a glucose control system 200a can include a controller 206a having an electronic processor 208a and a memory 214a that stores instructions 212a executable by the electronic processor 208a. The controller 206a and a pump 216 can be integrated into an ambulatory medical device (AMD) 202. The AMD 202 can have one or more pumps 216. The AMD 202 can include a transceiver 218a transceiver or wireless electronic communications interface 218a for wireless digital data communications with external electronic devices. When the instructions 212a stored in memory 214a are executed by the electronic processor 208a, the controller 206a can implement at least a portion of a control algorithm that generates dose control signals for one or more glucose control agents based on time-varying glucose levels of the subject (e.g., received from a glucose level sensor 110 that is in communication with the AMD 202, e.g., a medicament pump) and one or more control parameters. The dose control signals, when delivered to the pump 216, result in dosing operations that control the blood glucose of a subject. The pump 216 may be controlled by a pump controller. The pump controller receives the dose control signals and controls the operation of the pump 216 based on the received dose control signals. In some embodiments the pump controller may be integrated with the pump.

As shown in FIG. 2B, a glucose control system 200b can operate at least partially via execution of instructions 212b by an electronic processor 208b of an external electronic device 204 separate from the AMD 202. The external electronic device 204 can include a transceiver 218b capable of establishing a wireless digital data connection to the AMD 202, and a controller 206c can implement at least a portion of a control algorithm via execution of instructions 212b stored in memory 214b. When the instructions 212b stored in memory 214b are executed by the electronic processor 208b, the controller 206c can implement at least a portion of a control algorithm that generates dose control signals for one or more glucose control agents based on time-varying glucose levels of the subject and one or more control parameters. The dose control signals, when delivered to the pump controller of the pump 216, result in dosing operations that control the blood glucose of a subject. In some embodiments, the dose control signals are transmitted from the device transceiver 218b to the AMD transceiver 218a over a short-range wireless data connection 220. The AMD 202 receives the dose control signals and passes them to the pump 216 for dosing operations.

As shown in FIG. 2C, a glucose control system 200c can operate at least partially via execution of instructions 212c on an electronic processor 208c integrated with a remote computer 210, such as, for example, a cloud service. When the instructions 212c stored in memory 214c are executed by the electronic processor 208c, the controller 206d can implement at least a portion of a control algorithm that generates dose control signals for one or more glucose control agents based on time-varying glucose levels of the subject and one or more control parameters. The dose control signals, when delivered to the pump 216, result in dosing operations that control the blood glucose of a subject. In some embodiments, the dose control signals are transmitted from the remote computer WAN connection interface 224c to the AMD WAN connection interface 224a over an end-to-end wireless data connection 222. The AMD 202 receives the dose control signals and passes them to the pump 216 for dosing operations.

As shown in FIG. 2D, a glucose control system 200d can have two or more controllers 206a, 206c, 206d that cooperate to generate a dose control signal for dosing operations by the pump 216. A remote computer 210 can transmit or receive data or instructions passed through a WAN connection interface 224c via an end-to-end wireless data connection 222 to a WAN connection interface 224b of an external electronic device 204. The external electronic device 204 can transmit or receive data or instructions passed through a transceiver 218b via a short-range wireless data connection 220 to a transceiver 218a of an AMD 202. In some embodiments, the electronic device can be omitted, and the controllers 206a, 206d of the AMD 202 and the remote computer 210 cooperate to generate dose control signals that are passed to the pump 216. In such embodiments, the AMD 202 may have its own WAN connection interface 224a to support a direct end-to-end wireless data connection to the remote computer 210.

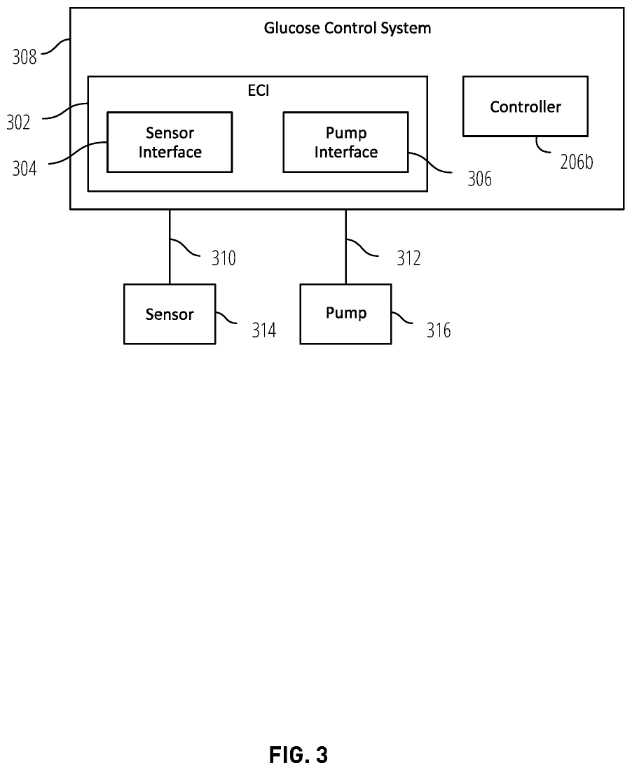

As shown in FIG. 3, in some embodiments, the glucose control system 308 includes circuitry that implements an electronic communications interface (ECI) 302 configured to send and receive electronic data from one or more electronic devices. The ECI includes a sensor interface 304 configured to receive a glucose level signal from a glucose level sensor 314 such as a continuous glucose monitor (CGM). Some CGMs generate the glucose level signal at fixed measurement intervals, such as five-minute intervals. The glucose level sensor 314 can be operatively connected to a subject in order to generate a glucose level signal that corresponds to a blood glucose estimate or measurement of the subject. The glucose level signal can be used by the controller 206b to generate a dose control signal. The dose control signal can be provided to a pump 316 via a pump interface 306. In some embodiments, the sensor interface 304 310 connects to the glucose level sensor 314 via a short-range wireless connection 310. In some embodiments, the pump interface 306 connects to the pump 316 via a short-range wireless connection 312. In other embodiments, the pump interface 306 connects to the pump 316 via a local data bus, such as when the controller 206b, the ECI 302, and the pump 316 are integrated into an AMD 100.

The controller can be configured to generate the dose control signal using a control algorithm that generates at least one of a basal dose, a correction dose, and/or a meal dose. Examples of control algorithms that can be used to generate these doses are disclosed in U.S. Patent Application Publication Nos. 2008/0208113, 2013/0245547, 2016/0331898, and 2018/0220942 (referenced herein as the “Controller Disclosures”), the entire contents of which are incorporated by reference herein and made a part of this specification. The correction dose can include regulatory or counter-regulatory agent and can be generated using a model-predictive control (MPC) algorithm such as the one disclosed in the Controller Disclosures. The basal dose can include regulatory agent and can be generated using a basal control algorithm such as disclosed in the Controller Disclosures. The meal dose can include regulatory agent and can be generated using a meal control algorithm such as disclosed in the Controller Disclosures. Additional aspects and improvements for at least some of these controllers are disclosed herein. The dose control signal can be transmitted to an infusion motor via the ECI 302 or can be transmitted to the infusion motor via an electrical conductor when the controller 206b is integrated in the same housing as the infusion motor.

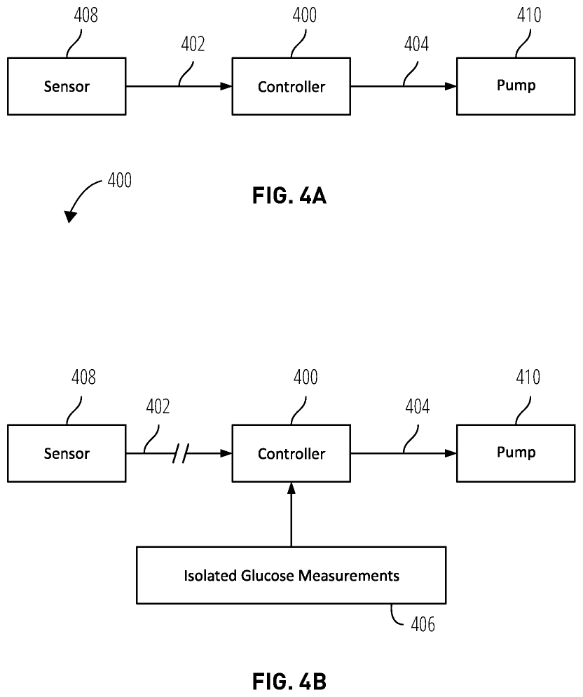

As shown in FIG. 4A, the controller 400 can be configured to operate in “online mode” during time periods when the controller receives a glucose level signal 402 from a glucose level sensor 408. In online mode, the control algorithm generates a dose control signal 404 that implements regular correction doses based on values of the glucose level signal 402 and control parameter s of the control algorithm. The pump 410 is configured to deliver at least correction doses and basal doses to the subject without substantial user intervention while the controller 400 remains in online mode.

As shown in FIG. 4B, the controller 400 can be configured to operate in “offline mode” during time periods when the controller does not receive a glucose level signal 402 from a sensor 408, at least during periods when the glucose level signal 402 is expected but not received. In offline mode, the control algorithm generates a dose control signal 404 that implements correction doses in response to isolated glucose measurements 406 (such as, for example, measurements obtained from the subject using glucose test strips) and based on control parameters of the control algorithm. The pump 410 is configured to deliver basal doses to the subject without substantial user intervention and can deliver correction doses to the subject in response to isolated glucose measurements 406 while the controller 400 remains in offline mode.

Example Ambulatory Medical Device (such as an Ambulatory Medicament Pump)

In some embodiments, the ambulatory medicament device (AMD) can be a portable or wearable device such as an ambulatory medicament pump (AMP) that provides life-saving treatment to a subject or subjects by delivering one or more medicaments (e.g., insulin and/or glucagon) to a subject or subjects. Some AMDs may continuously monitor the health condition of a subject using a sensor and deliver therapy such as one or more medicaments based on the health condition of the subject. For example, an ambulatory medicament pump (e.g., an insulin pump or a bi-hormonal pump) may monitor the blood glucose level in a subject using a Continuous Glucose Monitor (CGM) and adjust the dose or frequency of the medicament delivery (e.g., insulin or glucagon) accordingly. Certain ambulatory medicament devices may be worn by subjects constantly (e.g., all day), or for a large portion of the day (e.g., during waking hours, during sleep hours, when not swimming, etc.) to enable continuous monitoring of the health condition of the subject and to deliver medicament as necessary.

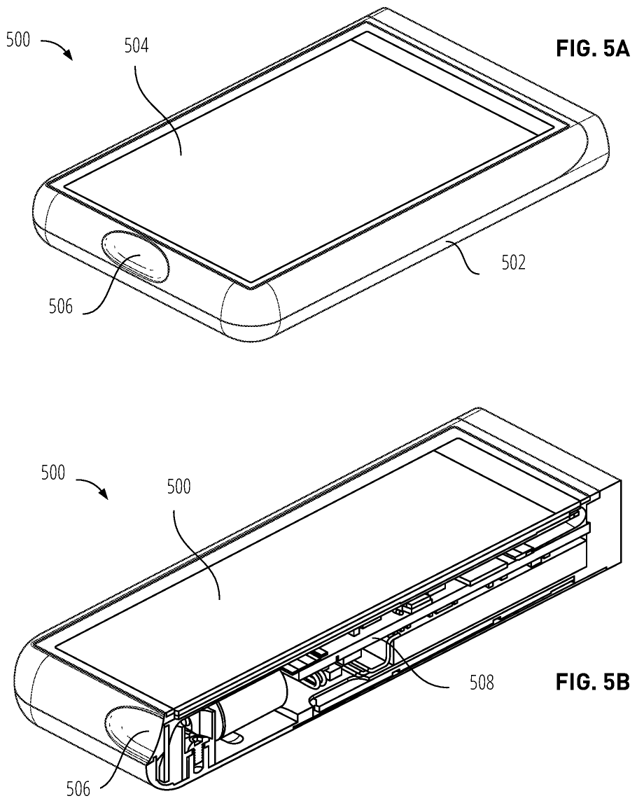

FIG. 5A illustrates a three-dimensional (3D) view of an example ambulatory medical device 500 (such as an ambulatory medicament pump) comprising a housing 502 with a wake button 506 and a touchscreen display 504. FIG. 5B is an illustration of a cross sectional view of the ambulatory medical device 500 shown in FIG. 5A. In this example, all the electronic modules 508 are included inside the housing, for example, as a single integrated electronic board. The wake button 506 may be any type of button (e.g., capacitive, mechanical) that registers an input generated by user interaction with the wake button 506 to generate a wake signal. In some embodiments, the wake signal is generated by a sensor (e.g., a biometric sensor such as a fingerprint reader or a retinal scanner, an optical or RF proximity sensor, and the like). In various embodiments, the wake signal may be generated by user interaction with the touch screen display 504 or with an alphanumeric pad (not shown). In some other examples, wake signal may be generated based on facial recognition or other biometric indicia. In yet other examples, the wake signal may be generated by a wireless signal such as RFID or Bluetooth signals or by detection of movement using one or more motion sensors such as an accelerometer. The wake button 506, if touched, pressed, or held for a certain period of time, may generate a wake signal that activates the touchscreen display 504. In some examples, touches on the touchscreen display 504 are not registered until the wake button activates the touchscreen display. In some such examples, the AMD remains locked from accepting at least certain types of user interaction or settings modification until a gesture (such as, for example, any of the gesture interactions described with reference to any of the embodiments disclosed herein) is received after the touchscreen display 504 is activated by the wake button 506. In some examples, after the touchscreen display 504 has been activated by the wake signal, a passcode may be required to unlock the touchscreen display. In some embodiments, the wake signal is generated by a sensor (e.g., a biometric sensor such as a fingerprint reader or a retinal scanner, an optical or RF proximity sensor, and the like). In various embodiments, the wake signal may be generated by user interaction with the touchscreen display 504 or with an alphanumeric pad (not shown). In some examples, a wake signal may be generated based on facial recognition or other biometric indicia. In some examples, the wake signal may be generated by a wireless signal such as a signal generated by an RFID system or Bluetooth signals received from an electronic device or by detection of movement using one or more motion sensors such as an accelerometer.

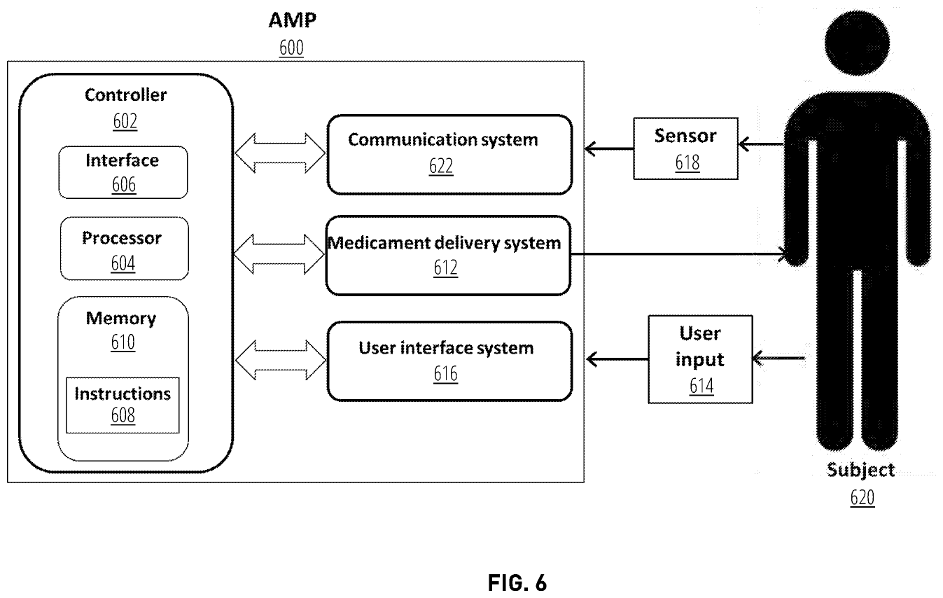

FIG. 6 illustrates different systems and sub-systems that may be included in an example AMP 600 (e.g., a blood glucose control system). As mentioned above, in some examples, the AMP may comprise glucose control system, such as a complete blood glucose control system. In some implementations, the AMP may include systems and sub-systems that can enable monitoring a subject's blood glucose level, managing the subject's diabetes, tracking a condition of the AMP 600, and/or communicating with one or more computing systems. For example, the AMP 600 may include a mono-hormonal or bi-hormonal medicament pump configured to administer one or more types of insulin and, in some cases, counter-regulatory agent (e.g., glucagon or other medicaments that can reduce or address hypoglycemia). As another example, the AMP 600 may include one or more alarm generators, transceivers, touchscreen controllers, display controllers, encryption sub-systems, etc. In some examples, two or more of the systems may be integrated together inside a single housing 502 (as shown in FIG. 5A and FIG. 5B). In some examples, one or more systems or sub-systems may be individual modules contained in separate housings that can communicate with other systems and/or the main unit via a wired or wireless communication link (e.g., Bluetooth). The AMP 600 may include a communication system 622, a medicament delivery system 612, a user interface system 616, and a controller 602 (or control system). In some embodiments, one or more systems may comprise one or more single purpose or multipurpose electronic sub-systems. In some such examples, one or more electronic sub-systems may perform procedures associated with different features of the AMP 600. In some other embodiments, one or more systems or sub-systems may comprise a non-transitory memory that can store machine readable instructions and a processor that executes the instructions stored in the memory. The memory may be a non-volatile memory, such as flash memory, a hard disk, magnetic disk memory, optical disk memory, or any other type of non-volatile memory. Further, types of memory may include but are not limited to random access memory (“RAM”) and read-only memory (“ROM”). In some such examples, a system can be programed to perform different procedures each implemented based on a different set of instructions.

The controller 602 may include one or more processors 604, a memory 610 that may comprise one or more non-transitory and/or non-volatile memories and an interface 606 that enables data and signal communication between the controller 602 and other systems of the AMP (e.g., communication system 622, medicament delivery system 612 or user interface system 616). The memory 610 may be divided into two or more memory segments. The main memory 610 may exchange data with sub-systems within the controller 602 as well as other systems (e.g., via the interface 606). The memory 610 may store data while the controller 602 is powered or unpowered. The processor 604 may be any type of general-purpose central processing unit (“CPU”). In some embodiments, the controller may include more than one processor of any type including, but not limited to complex programmable logic devices (“CPLDs”), field programmable gate arrays (“FPGAs”), application-specific integrated circuits (“ASICs”) or the like. The interface 606 may include data transfer buses and electronic circuits configured to support data exchange among different sub-systems within the controller 602. In some examples, in addition to data exchange between any of the systems and the controller 602, the interface 606 may support data and signal exchange among other systems of the AMP 600.

The interface 606 may include a plurality of interconnected electronic modules for signal conditioning and signal conversion (e.g., A-to-D or ADC conversion and D-to-A conversion or DAC conversion) configured to support communication and data exchange between different systems. For example, the interface 606 may convert an analog signal received from the communication system 622 and convert it to a digital signal that can be processed by the controller 602. As another example, the interface 606 may receive a digital control signal and convert it to a dose signal (e.g., an analog signal) that can be transmitted to the medicament delivery system 612, for example, to control one or more infusion pumps included in the medicament delivery system 612.

In some embodiments, the medicament delivery system 612 may comprise one or more infusion pumps configured to deliver one or more medicaments (e.g., insulin or glucagon) to a subject 620 and a pump controller that may activate the infusion pumps upon receiving does control signals. In some examples, the medicaments may be stored in one or more medicament cartridges that may be housed in or inserted into the medicament delivery system 612. In some examples, the medicament delivery system 612 may include electronic and mechanical components configured to control an infusion pumps based on signals received from controller 602 (e.g., via the interface 606).

The user interface system 616 may include a display to show status information about the AMP 600 and/or medicament. For example, the status information may include medicament type, delivery schedule, software status, and the like. The display may show graphical images and text using any display technology including, but not limited to OLED, LCD, or e-ink. In some embodiments, the AMP 600, may include a user interface (e.g., an alphanumeric pad) that lets a user provide information or interact with the AMP 600 to modify the settings of the AMP 600, respond to request for certain actions (e.g., installing a software) and the like. The alphanumeric pad may include a multitude of keys with numerical, alphabetical, and symbol characters. In different embodiments, the keys of the alphanumeric pad may be capacitive or mechanical. The user may be a subject 620 receiving medicament or therapy, or may be an authorized user, such as a clinician or healthcare provider, or a parent or guardian of the subject 620. In some other embodiments, the AMP 600 may include a touchscreen display that produces output and accepts input enabling a two-way interaction between the user and the AMP 600. The touchscreen display may be any input surface that shows graphic images and text and registers the position of touches on the input surface. The touchscreen display may accept input via capacitive touch, resistive touch, or other touch technology. The input surface of the touchscreen display can register the position of touches on the surface. In some examples, the touchscreen display can register multiple touches at once. In some embodiments, the keypad may be a display of a keypad. For example, an alphanumeric pad comprising user-selectable letters, numbers, and symbols may be displayed on the touchscreen display. In some examples, the touchscreen may present one or more user-interface screens to a user enabling the user to modify one or more therapy settings of the ambulatory medicament device.

In some examples, a user-interface screen may comprise one or more therapy change control elements (e.g., displayed on the touchscreen display) enabling a user or the subject to access therapy change controls and modify therapy settings by interacting with these control elements. For example, the user can modify the therapy settings by changing one or more control parameters using the corresponding therapy change control elements. In some embodiments, a therapy control parameter may be any parameter that controls or affects the volume, duration and/or the frequency of medicament doses delivered to the subject.

In some embodiments the communication system 622 may include one or more digital data interfaces to send or receive digital data via a wired or a wireless link. For example, the communication system may include one or more wireless transceivers, one or more antennas, and/or one or more electronic systems (e.g., front end modules, antenna switch modules, digital signal processors, power amplifier modules, etc.) that support communication over one or more communication links and/or networks. In some examples, each transceiver may be configured to receive or transmit different types of signals based on different wireless standards via the antenna (e.g., an antenna chip). Some transceivers may support communication using a low power wide area network (LPWAN) communication standard. In some examples, one or more transceivers may support communication with wide area networks (WANs) such as a cellular network transceiver that enables 3G, 4G, 4G-LTE, or 5G. Further, one or more transceivers may support communication via a Narrowband Long-Term Evolution (NB-LTE), a Narrowband Internet-of-Things (NB-IoT), or a Long-Term Evolution Machine Type Communication (LTE-MTC) communication connection with the wireless wide area network. In some cases, one or more transceivers may support Wi-Fi® communication. In some cases, one or more transceivers may support data communication via a Blue tooth or Blue tooth Low Energy (BLE) standard. In some examples, one or more transceivers may be capable of down-converting and/or up-converting a baseband or data signal from and/or to a wireless carrier signal. In some examples, the communication system 622 may wirelessly exchange data between other components of the AMP 600 (e.g., a blood glucose level sensor), a mobile device (e.g., smart phone, a laptop and the like), a Wi-Fi network, WLAN, a wireless router, a cellular tower, a Bluetooth device and the like. The antenna may be capable of sending and receiving various types of wireless signals including, but not limited to, Bluetooth, LTE, or 3G.

In some examples, the communication system 622 may support direct end-to-end communication between the AMP 600 and a server or a cloud network. In some examples the AMP 600 may communicate with an intermediary device (e.g., a smart phone or other mobile devices, a personal computer, a notebook, and the like). In some embodiments, the AMP 600 may include an eSIM card that stores information that may be used to identify and authenticate a mobile subscriber. The eSIM card may enable the AMP 600 to function as an Internet of Things (IoT) device that can communicate over a network that supports communication with IoT devices. In other embodiments, the AMP 600 may be configured to transmit data using a narrowband communication protocol such as 2G or EDGE. Using the cellular connection, the AMP 600 may be paired with a mobile device at inception and permit real-time data access to the AMP 600 by a healthcare provider. In certain implementations, the AMP 600 may include a geolocation receiver or transceiver, such as a global positioning system (GPS) receiver. As previously stated, each of the AMPs described herein may include one or more of the embodiments described with respect to the other AMPs unless specifically stated otherwise. In some embodiments the communication system 622 may include a Near Field Communication (NFC) sub-system that enables contactless data exchange between the AMP 600 and an electronic device located in the vicinity of the AMP 600.

### Example Operation of the AMP

In some embodiments, the AMP 600 may continuously, periodically, or intermittently receive data related to a health condition of the subject (e.g., blood glucose level, blood glucose trend, heart rate, body movement indicia, etc.). This information may be encoded to a signal provided to AMP 600 by a sensor 618. In some such embodiments, the sensor 618 can be a continuous glucose monitor (CGM) that is connected to the AMP 600 via a wired or wireless link (e.g., a link based on or implementing the Bluetooth® standard). In some examples, a CGM may be a wearable biomedical sensor that measures a blood glucose level in the interstitial fluid. In some examples, the signal sent by the sensor 618 may be received by the communication system 622 and transmitted to the controller 602 where the signal may be analyzed to determine whether to deliver medicament to the subject 620. In some examples, a second communication system may be included in the AMP 600 to communicate with the sensor 618. If it is determined that medicament should be administered to the subject, the controller 602 may determine the dosage and/or type of medicament to administer to the subject. The determination of medicament dosage and/or type may be based at least in part on the information received from the sensor 618. The controller 602 may generate a dose control signal and send the dose control signal to the medicament delivery system 612 to initiate medicament delivery to the subject. In some examples, the dose control signal may be received by a pump controller that controls operation of an infusion pump.

In some embodiments, the controller 602 may perform one or more procedures using the processor 604 (or a plurality of processors) that execute instructions 608 stored in the memory 610 of the controller 602. These procedures may include, but are not limited to, determining the need for delivering medicament, determining the type of medicament and/or a dose size of medicament, determining the rate of delivery during a therapy session, providing information (e.g., device status, next delivery time, level of certain analytes in the subject's blood, and the like) via the user interface system 616, processing the data received from the sensor 618, or managing access to control parameters (e.g., by controlling one or more therapy change controls that may be provided by the user interface system 616).

In some embodiments, the AMP 600 may deliver multiple types of therapy. The type of therapy to be delivered may be determined automatically by the controller 602 or may be selected by the user. For example, the AMP 600 may deliver the therapy of infusing insulin into a user and may also deliver glucagon into the user. In some examples, the user interface system 616 may allow the user to select the type of medicament infused to the subject 620 during therapy (e.g., insulin, glucagon or both). In other embodiments, other hormones, medicaments, or therapies may be delivered. In some examples, the controller 602, may determine the type of medicament that is delivered to the subject 620 base at least in part on the data received from the communication system 622.

### Communication and Networking

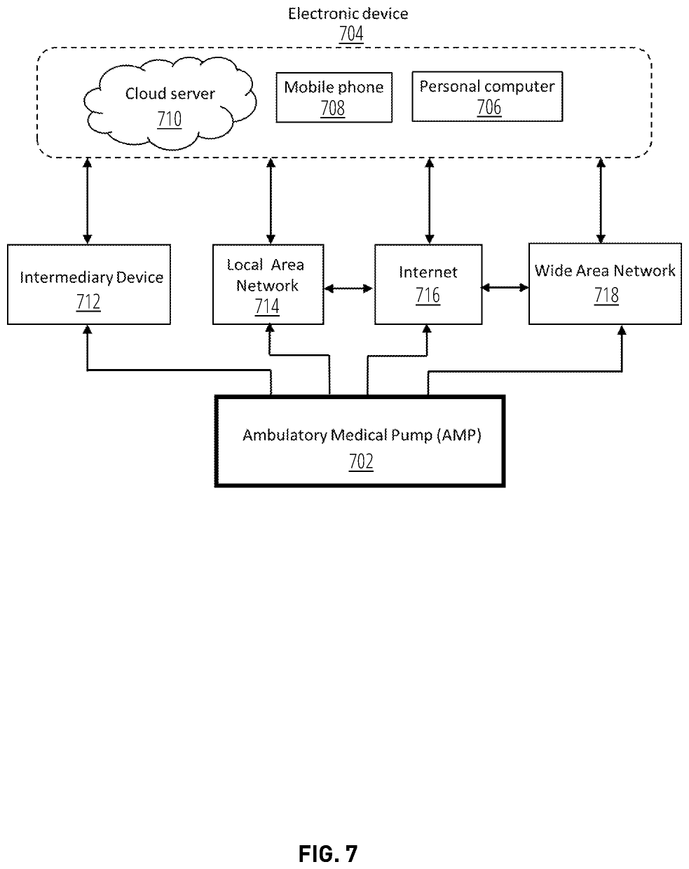

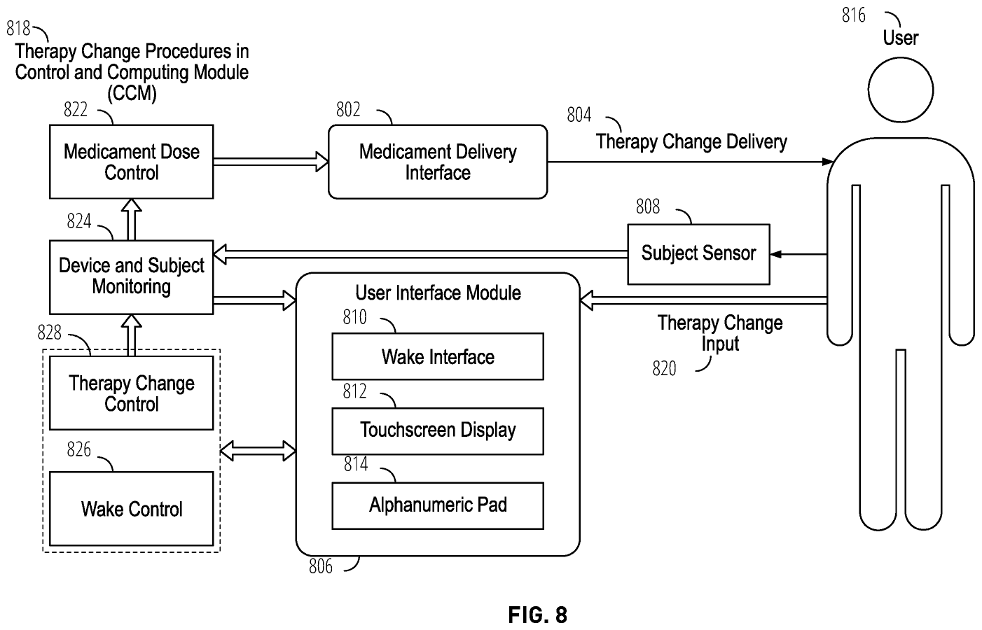

FIG. 7 illustrates various methods, and data links or communication paths that AMP 702 may use to communicate (e.g., exchange digital data) with an electronic device 704. The communication may include obtaining application updates, sending and/or receiving therapy data and/or therapy reports, receiving passcodes or access codes, sending a request (e.g., request for a safe access level or a request for a therapy report), receiving values of control parameters, and the like. The electronic device 704 can be a cloud server 710, a mobile phone 708 (e.g., a mobile phone of a user, the subject 620, a physician, and the like), or a personal computer 706 (e.g., a laptop, desktop, tablet, etc. of a user, the subject 620, a physician, and the like). In some examples, the cloud server 710 may be part of a data center (e.g., the data center of a healthcare provider).

In some embodiments, the AMP 702 may establish a connection (e.g., using the communication system 622) with the electronic device 704 through an intermediary device 712 (e.g., a smart phone or other mobile device, a personal computer, a notebook or the like). In some examples, the AMP 702 may communicate with the electronic device 704 through a local area network 714 (LAN) and/or through a Wi-Fi connection. Alternatively, or in addition, the AMP 702 may establish a communication connection to the electronic device 704 via a wide area network 718 (WAN). In some examples, the communication between the AMP 702 medical device and the electronic device may be encrypted.

In some embodiments, the AMP 702 may establish a direct end-to-end communication connection over a wide area network 718 (e.g., a cellular network) with the electronic device 704. In some cases, a direct-end-to-end communication connection may be a connection that does not involve a local device, a device that is accessible by the user or the subject (besides the AMP 702), a Wi-Fi network, a short range wireless link (e.g., Bluetooth), or the like. In some such cases, the direct end-to-end communication may pass through one or more wireless systems (e.g., receivers, transmitters or antenna) of a wide area network 718. In some examples, the electronic device 704 may establish the end-to-end connection by receiving a public key from the AMP 702. In some examples, the public key and a private key stored in the electronic device 704 can be used to permit the electronic device 704 to decrypt data transmitted by the AMP 702. In some implementations, the electronic device 704 may establish a direct end-to-end data connection with the AMP 702 based on receiving a device identifier associated with the AMP 702. The device identifier may be a unique identifier specific to the AMP 702. In some other implementations, establishing the direct end-to-end data connection may include determining that the AMP 702 is permitted to communicate with the electronic device 704 based at least in part on the device identifier. In some examples, the device identifier may be initially provided to electronic device 704 prior to provisioning of the AMP 702 to the subject. For example, the device identifier may be initially provided to the networked-computing environment as part of a manufacturing process for manufacturing the AMP 702. In some examples, the device identifier may include or may be based on one or more of an Internet Protocol (IP) address, a Media Access Control (MAC) address, a serial number, or a subject identifier of a subject that receives therapy from the AMP 702. In some cases, the subject or a user may establish or initiate establishing the direct end-to-end data connection with the electronic device 704. In some cases, the direct end-to-end data connection may be initiated or established without any action by the subject or the user. For example, the direct end-to-end data connection may be established automatically at particular times and/or when the AMP 702 is in a particular location. In some such cases, this automatic connection may occur using information supplied to the AMP 702 at a time of manufacture, shipment, sale, or prescription to the subject. Alternatively, or in addition, a subject or other user may configure the AMP 702 to automatically connect to the electronic device 704 at particular times and/or locations. In some cases, the wide area network may include, or may communicate with, the Internet 716.

In some embodiments, the AMP 702 may be configured to communicate via the wide area network during manufacture or prior to being provisioned to the subject. For example, a manufacturer can register the AMP 702 with a wireless wide-area network provider (e.g., T-Mobile or Verizon) and provide an International Mobile Equipment Identity (IMEI) number or serial number for the AMP 702 to the network provider. Moreover, fees can be negotiated between the manufacturer and the network provider or between the subject's health insurance and the network provider. Similarly, fees may be paid by the manufacturer or health insurance provider, or other entity, without subject involvement. Thus, the subject's AMP 702 may be configured to communicate via the network of the network provider without any action by the subject or the user. In some cases, the subject may be responsible for obtaining wireless service to connect the AMP 702 to a wide area network 718 (e.g., a cellular network).

In some examples, the AMP 702 may be pre-registered or authenticated with a computing network of the cloud services provider as part of the manufacturing process or before AMP 702 is provided to the subject. This enables the AMP 702 to communicate over the wide area network with the computing system of the cloud services provider from day one without any or with minimal configuration by the subject. In some cases, a user, such as a healthcare provider may register or associate the AMP 702 with the subject at the computing network of the cloud services provider.

In some embodiments, the AMP 702 may use a whitelist, or approved list, that identifies via a unique identifier (e.g., via an IP address, a MAC address, or a URL) one or more permitted cloud servers 710 or computing systems of the cloud computing system that the AMP 702 is permitted to access. By restricting access to an approved set of computing systems, the risk of malicious actors accessing the AMP 702 is reduced. Moreover, in some cases, the AMP 702 may include a blacklist, or restricted list, that identifies systems the AMP 702 is not permitted to access. The blacklist may be updated as more restricted or unsafe websites, network accessible systems, or computing systems are identified. Similarly, the whitelist may be updated over time if approved systems are added or removed.

Further, the cloud computing service may have a whitelist, or approved list, that uses unique identifiers to specify AMP 702 and/or other computing systems (e.g., remote display systems) that are permitted to communicate with the cloud computing system. Moreover, as with the AMP 702, the cloud computing service may have a blacklist or restricted list that identifies AMPs, or other computing devices, that are not permitted to access the cloud computing services. An AMP 702 may be added to the restricted list if it is decommissioned, damaged, or is no longer in possession of the subject. It may be desirable to remove an AMP's access to the cloud computing service to help protect private or personal data of a subject. Advantageously, establishing a connection based on a whitelist may enhance the security of the communication link established between AMP 702 and the cloud server 710 or other computing systems. In addition to the identifiers that identify permitted computing systems for access by the AMP 702 and/or permitted AMPs for access by a cloud or networked computing service, the whitelist may include any information that may facilitate access to the systems identified on the whitelist. For example, the whitelist may include access information (e.g., usernames, passwords, access codes, account identifiers, port identifiers, a shared secret, public keys, etc.). It should be understood that the whitelist may include different information depending on whether the whitelist is publicly accessible, accessible by only the AMP, accessible by authorized users or devices, etc. For example, a publicly accessible whitelist or a whitelist accessible by more than one authorized system or user may not include passwords or access codes.

In some cases, the AMP 702 may use a whitelist that identifies via, for example, a unique identifier (e.g., via an IP address, a MAC address, or a URL) permitted cloud servers or computing systems. In some examples, the electronic device 704 may have a whitelist that uses unique identifiers to specify AMP 702 and/or other computing systems (e.g., remote display systems) that are permitted to communicate with the electronic device 704. The whitelist may be stored in a memory of AMP 702 and/or in a memory of a trusted computing device that is accessible by the AMP 702. The trusted computing device can include any computing device that a manufacturer of the AMP 702 has identified as trusted. Alternatively, or in addition, the trusted computing device can include any computing device that a subject or user that care for the subject (e.g., parent, guardian, or healthcare provider) has identified as a trusted computing device that is designated to store the whitelist. In some examples, the whitelist may be configured during manufacture of the AMP 702. For example, the whitelist may be configured with connection information to establish communication with one or more computing systems of a networked-computing environment. In some examples, the AMP 702 may be configured to execute the specific computer-executable instructions to at least obtain an address of a computing system from the whitelist and to establish a direct end-to-end data connection to the computing (e.g., a computing system in the networked-computing environment), via a wireless wide area network using the address. In some embodiments, the AMP 702 may be configured to execute the specific computer-executable instructions to at least receive a public key from the computing system of the networked-computing environment.

In some embodiments, a portion of the communication system 622 of the AMP 702 may be enabled or disabled by the controller 602. In some cases, upon receiving a trigger, the controller 602 may maintain wireless data communication between the AMP 702 and the sensor 618 but disable wireless communication with all other electronic devices. In some examples, the trigger can be a signal received from the user interface system 616 indicating a user interaction with a user interface (e.g., a touchscreen display). For example, a user interface element may be provided on a touchscreen display to activate an “airplane mode.” In some embodiments, the trigger may be a signal received from a location sensor. For example, the controller may automatically disable wireless communication signals that are not associated with the sensor 618, upon receiving a location signal from a location sensor (e.g., a GPS) indicating that the user is an airplane.

In some embodiments, the controller 602 may switch on one or more wireless communication sub-systems of the communication system 622 upon determining that a condition is satisfied by the therapy data (e.g., glucose level received from the sensor 618). In some embodiments, the controller 602 may switch on one or more wireless communication sub-systems of the communication system 622 based on a location of the user or the subject determined by a location sensor.

In some embodiments, one or more settings of the AMP 702 can be stored in an electronic device automatically or upon receiving a request from a user. For example, using a user interface of the AMP 702, the user may send a request to save one or more settings of the AMP 702 in a remote electronic device (e.g., a cloud server) via a wireless digital data link. As another examples, the AMP 702 may automatically store its current settings in a remote electronic device (e.g., a cloud server), by establishing wireless data connection with the remote electronic device. In some examples, a user may provide instructions to the AMP 702 (e.g., via user interface or a wireless link), so that the AMP 702 periodically (e.g., every week, every month) stores one or more settings of the AMP 702 in a remote electronic device.

In some embodiments, a one-time passcode may be provided to the user or the subject to enable connecting the AMP 702 to a remote electronic device (e.g., a cloud server), in order to download a device state to the AMP 702. For example, when a user obtains a new AMP, the user data and customized settings, which were previously stored in the remote electronic device, may be downloaded to the new AMP. The onetime pass code can be requested from a computer or a mobile phone, for example, using an application program configured to “set up a pump”.

In some cases, the new AMP may be provided to the user with limited functionality (e.g., the pump motor may not be enabled) while the prescription verification process is proceeding. Once the prescription is verified, the required functionalities and settings associated with prescription, may be enabled.

In some, examples the eligibility of a user for downloading the personal data and/or the AMP state (e.g., therapy settings) may be confirmed based on the AMP's serial number.

### Modifying Therapy Settings

In some embodiments, AMP 702 may allow a user or the subject 620, to modify a therapy setting of the AMP 702. The therapy setting may comprise values or selections related to one or more control parameters that control the dose control signal generate by the controller 602. For example, the AMP 702 may provide one or more therapy change controls in a user interface that may receive a user input 614 to modify one or more control parameters of the AMP 702. In some embodiments, the therapy change controls can be therapy change control elements provided in a therapy change user interface (e.g., a touchscreen display) and the user input 614 can be received in response to a user interaction with a therapy change control element.

In certain embodiments, the user may wake the AMP 702 from a sleep state or unlock the AMP 702 by, for example, interacting with a wake interface. When the AMP 702 is in a sleep state, the user interface may not receive the user input 614 or user input signals corresponding to user input. Waking the AMP 702 may include activating a touchscreen interface or presenting a lock screen to a user. Further, waking the AMP 702 may include waking the touchscreen controller such that it can receive user input or user input signals corresponding to user input. The wake interface can include one or more of the additional user interfaces mentioned above that are configured to generate and provide a wake input (or wake signal) to the controller 602 when detecting a pre-set user interaction. Alternatively, or in addition, the wake interface can be any type of wake interface element of the AMP 702 that a user can interact with to wake at least a feature (e.g., a touchscreen interface) of the AMP 702. For example, the wake interface element can be a physical button (e.g., a push button, a slide button, etc.), a capacitive element, a resistive element, or an inductive element. In some cases, the wake interface element can be or can include a biometric element, such as a fingerprint reader, an iris scanner, a face detection scanner, etc. In some cases, the AMP 702 may wake in response to detection of a movement or motion. For example, a determination that the ambulatory medicament device is being moved with a particular motion or within a line of sight or a visual range of a user may cause the AMP 702 to awaken or cause the AMP to awake the touchscreen interface of the AMP 702. The AMP 702 may determine that the AMP 702 is being moved within a line of sight of the user based on the type of motion and/or the detection of a user's eyes via, for example, an iris scanner or a camera.

In some examples, the user input 614 can be an input provided by the subject 620 to change a therapy that is currently being delivered to the subject 620. For example, the user input 614 may cause the insulin or glucagon infusion pump to start infusing an amount of insulin or glucagon into the 618. In some examples, the user input 614 provided by the subject 620, may affect the therapy delivery at future time. In some examples, the user input may modify the rate of insulin or glucagon infusion into the user subject 620. The user input 614 may also cancel insulin or glucagon infusion into the subject 620 from the insulin or glucagon infusion pump. In some cases, the user input 614 is a request to change a control parameter. The control parameter may be changed in response to the request. Alternatively, or in addition, a confirmation action (e.g., a swipe gesture or an interaction with a physical or digital button on the touchscreen) confirming the requested control parameter change may be required before the control parameter is changed.

In some cases, when a wake action is detected by the wake interface, a wake input is sent to the 602, which may imitate or perform a wake control procedure to wake/unlock the user interface (e.g., a touchscreen display). In some cases, the controller 602 may use the wake controller to perform the wake procedure.

When in the wake and/or unlocked state, a user may interact with a touchscreen display, alphanumeric pad or other types of user interfaces that may be provided by user interface system 616 in a the user interface, to obtain access to therapy change user interface.

The therapy change user interface may be activated by a first user interaction with the user interface (e.g., touchscreen display). When the first user interaction is detected, the user interface system 616 may send an input signal to the controller 602 to determine if the first user interaction relates to a therapy change request or a control parameter change request. In some cases, the controller 602 may determine whether the first user interaction corresponds to a request to change a control parameter, or a request to access a control parameter change interface. If it is determined that the first user interaction satisfies a set of predefined conditions, the controller 602 may send a signal to the user interface system 616 to activate the therapy change user interface.

In some embodiments, the type of therapy change user interface and/or the available therapy change selections included in the user interface may depend on the user interaction. For example, in response to one of two user interactions, the controller 602 may send one of two signals to the user interface system 616. The therapy change user interface may unlock one of two different therapy change user interfaces that result in different options of therapy change selections for the subject 620. In an implementation of this example, a therapy change selection to make a significant therapy change, such as dramatically (e.g., more than a magnitude, or more than 3 change increments) increase the rate of insulin or glucagon infusion rate, may require a user interaction that is different from the user interaction that may be required for an insulin or glucagon infusion at a normal or prescribed rate, or a smaller change to the control parameter. In some examples, the user interaction may be a simple interaction (e.g., a simple gesture or unlock gesture interaction) that unlocks a therapy change user interface with therapy change selections that are limited in magnitude size. Another user interaction may be a complicated interaction (e.g., a series of complex gestures) that unlocks a therapy change user interface with therapy change selections that have no limits. An example of this implementation may be useful for child users. The child user may perform the first or simpler gesture that is made up of a series of simple inputs to unlock therapy change selections that are limited. An adult user may perform the second or more complex gesture that is made up of a series of complex inputs to unlock the therapy change user interface with therapy change selections that have no limits. In some cases, a simple interaction may include an interaction that is capable of being performed by a person below a particular age (e.g., a child), above a particular age (e.g., a senior citizen), or associated with a particular level of maturity or intelligence (e.g., a child or an individual with a learning disability). In contrast, a complex interaction may include an interaction that can be performed by an average adult or person that is above a particular minimum age (e.g., older than a child of a particular age), above a particular maximum age (e.g., younger than a senior citizen), or is not associated with a learning disability.

Once activated, the therapy change user interface generated by the user interface system 616 may provide one or more therapy change control elements that enable the user to modify the one or more settings of the AMP 702. In some examples, the therapy control element may include any type of user interface screen on the touchscreen, or other type of user interface in the non-touchscreen context, that enables or permits a user to change a configuration of the AMP 702. This change in configuration of the AMP 702 may relate to a change in the therapy provided or in the detection of a triggering event that causes therapy (e.g., medicament delivery) to be provided to a subject. For example, the change in configuration may include a selection between one or more hormones that regulate blood sugar level (e.g., insulin or glucagon) of a user, an amount of the one or more hormones that regulate blood sugar level of the user, a rate of delivery of the one or more hormones, a threshold for determining when to deliver the one or more hormones, a change in an estimated blood absorption rate of the one or more hormones, and the like. In some examples, the therapy control element may include any type of user interface screen on the touchscreen, or other type of user interface in the non-touchscreen context, that enables or permits a user to change one or more control parameters of the AMP 702 that control the therapy delivery. In some cases, a simple interaction may include an interaction that is capable of being performed by a person below a particular age (e.g., a child), above a particular age (e.g., a senior citizen), or associated with a particular level of maturity or intelligence (e.g., a child or an individual with a learning disability). In contrast, a complex interaction may include an interaction that can be performed by an average adult or person that is above a particular minimum age (e.g., older than a child of a particular age), above a particular maximum age (e.g., younger than a senior citizen), or is not associated with a learning disability.

In some cases, a change to the setting (e.g., control parameters or configuration of the AMP) of the AMP 702 is automatically and/or instantly recognized or implemented by the AMP 702, and/or transmitted to the AMP 702. In some cases, a confirmation of the change may be required before the setting change is implemented by or transmitted to the AMP 702.

A confirmation of the change may be received in response to a second user interaction with a user interface (e.g., touchscreen display). When the second user interaction is detected, the user interface system 616 sends an input signal to the controller 602. If it is determined that the second user interaction satisfies a set of predefined conditions, the controller 602 implements the change to the configuration of the AMP 702.

The first and/or second user interactions may include the selection of an icon, a series of taps or inputs, one or more gestures (e.g., a linear swipe, an arcuate swipe, a circular swipe, or other simple or complex movement across the touchscreen), performing a pattern or sequence on the touchscreen (e.g., drawing an image), a multi-touch or multi-input interaction, a combination of the foregoing, or any other type of interaction with a touchscreen, or portion thereof. The series of inputs may be any combination of touch movements, touch points, numerical characters, alphabetical characters, and other symbols. Gesture interactions can be guided by visual indicia displayed or printed on the AMP 702. In some embodiments, the visual indication can include animations that suggest or guide user interactions with a touchscreen. For example, the first user interaction can include an arcuate swipe around at least a portion of a generally circular icon or logo. In some examples, the first and/or second user interactions may include a predetermined sequence of numerical and/or alphabetical inputs. In some examples, a series of multiple inputs, the range of parameters for an input may be dependent on other inputs in the series. For example, required start position of a touch movement may be dependent on the position of the previous touch movement. The time that the series of inputs are entered may also be a part of the range of parameters. For example, a series of inputs may need to be entered in no less than 3 seconds or more than 3 seconds, and no more than 15 seconds or less than 15 seconds.

Further, one or more of the interactions may include interacting with a sensor, such as an optical sensor (e.g., visible light or IR sensor), biometric sensor (e.g., a fingerprint or retinal scanner), a proximity sensor, a gyroscope, or a combination of accelerometer and gyroscope, and the like. Also, in some cases, the second user interaction may be received through a wireless signal such as RFID or Bluetooth. In some embodiments, the second user interaction may include receiving a selection of a user interface element (e.g., a checkbox). Further, the second user interaction may correspond to a medicament type, such as insulin or glucagon. In some cases, the second interaction may correspond to receiving a particular sequence of numerical inputs in order to confirm the therapy change selection.

The type of user interaction that unlocks the touchscreen, provides access to a configuration screen, and/or confirms a change to the configuration of the AMP 702 may be the same or may differ.

In some examples, the system may have a time-out feature associated with a particular length time period. This particular length time period may be of any length. For example, the time period until a time-out occurs may be 30 seconds, a minute, 5 minutes, etc. Further, the length of the time period associated with a time out may be programmable. In some such cases, if no interaction occurs during the particular length time period, a time-out occurs. When a time-out occurs, a process for modifying an AMP 702 configuration may be cancelled. If the user elects to reattempt to modify the AMP 702 configuration after occurrence of a time-out, the process may be restarted. In some cases, the user interface will turn off upon occurrence of a time-out, and as stated above, the therapy change request process may be required to start again. In one implementation of the time-out, if no interaction occurs for more than 30 seconds after the system is waked/unlocked before the second user interaction is received by the user interface, the user interface will be deactivated.

In some implementations, once a change or modification to the therapy setting (e.g., a change in control parameters or configuration of the AMP 702) is confirmed, implemented, or transmitted, the AMP 702 may begin operating based on the modified setting selected and/or provided by the user.

In some cases, operating with the modified setting may include triggering therapy delivery based on the new setting or providing therapy based on the new setting. For example, the AMP 702 may generate a dose control signal based at least in part on the modified configuration or control parameter or may detect a trigger based at least in part on the modified configuration or control parameter that leads to the provisioning of therapy.

In some cases, AMP 702, or a control device that enables a user to modify a configuration of the AMP 702, may have a timeout feature. The timeout feature may cause the AMP 702 or the control device to enter a sleep or locked state after a period of inactivity by the user. In some cases, the timeout feature may cause the AMP 702 or the control device to enter a sleep or locked state after a particular period regardless of whether the user is interacting with the ambulatory medicament device or control device. In some cases, the timeout feature may cause the user interface (e.g., a touchscreen display) to become inactive or enter a lock state. Thus, a user may have a limited period to modify the configuration of the AMP 702.

In some examples, the therapy change made by a user may trigger the delivery of a medicament according to the therapy change received and confirmed by a user. This therapy change delivery may occur after a set time period from receiving the confirmation.

In some embodiments, the AMP 702 may allow the user to provide a therapy change and then cancel the therapy change. The user may provide the therapy change by modifying one or more control parameters of the AMP 702. The user may unlock a touchscreen display using a wake action and get access to a therapy change user interface (e.g., using a first gesture), where one or more therapy change control elements may be displayed. Next, an indication of a modification to a therapy control element may be received by the user interface followed by a confirmation of the modification made (e.g., a second gesture). In response to receiving an indication and confirmation of a modification to a therapy control element, the corresponding control parameter may be changed from a first setting to a second setting. In some examples, once the change is implemented, the user may decide to cancel it or revert to a prior setting. For example, the user may determine that the change is erroneous or does not provide the anticipated level of care. In some such cases, the user may provide a third gesture on the touch screen. In response to receiving the third gesture from the user interface, the therapy change procedure may restore the modified control parameter to the first setting.

In some examples, the third gesture may be a restore gesture. In some cases, the restore gesture may be a swipe gesture. In some examples the swipe gesture may be performed near or in a region of the therapy change user interface that is occupied by the therapy control element (or a particular portion thereof). An example of a restore swipe gesture may be a gesture performed from a starting swipe position to an ending swipe position located closer to a left edge of the touchscreen than the starting swipe position. For instance, a user may position a finger at a point on the touchscreen and drag the finger across at least a portion of the touchscreen towards a left edge (e.g., reminiscent of a back arrow). It should be understood that other gestures are possible to indicate a restore gesture. In some cases, a user can define the gesture to be used as a restore gesture. In some embodiments, the restore gesture is received on a different user interface screen than a therapy change user interface wherein one or more therapy control element are provided. In various examples, the restore gesture is performed in the opposite direction from a therapy change confirmation gesture that confirms the modification to the therapy control element.

In some examples, in order to cancel a therapy change request, the restore gesture has to be provided within a set time period after the confirmation gesture is received by the user interface. In some such examples, during the set time period one or more dose control signals may be provided to the medicament delivery interface resulting in one or more therapy change deliveries. In some cases, the restore gesture can be received at any time after the confirmation gesture or therapy change. In some cases, the restore gesture restores the control parameter modified during a therapy change to an immediately preceding value. In some cases, the restore gesture restores the control parameter to a value designated as a restore value (e.g., a default value or other designated restore value) or to a most recent value that maintained the subject's blood glucose level with a target setpoint range. The designated restore value may be specific to a subject or AMP 702 or may be determined based on clinical data for a set of subjects. The set of subjects may be subjects that share certain characteristics with the subject using the AMP 702. For example, the set of subjects may be of the same gender, similar age range, similar severity of diabetes, etc.

In some cases, the system may allow the user to modify a therapy change before confirmation. In these cases, the user may modify a therapy control element for a second time to change the corresponding control parameter from a second setting to a third setting.

In some examples, the third setting may be the same as the first setting. In some cases, the first setting or the third setting may be a default setting. In some cases, the first setting or the third setting may be a restore setting.

In some examples, the user may be able to cancel a therapy change delivery after confirming the therapy change and before a therapy delivery based on new settings. In some such examples, an alert may notify the user that a therapy delivery based on new settings will occur shortly.

In some examples, the user may be able to cancel a therapy change delivery triggered based on therapy change made by the user. In these examples, the user may get access to the user interface using a wake action and provide a gesture to cancel the ongoing therapy delivery based on a therapy change delivery.

### Therapy Data and Therapy Report

In some examples, AMP 702 may establish a communication with an electronic device 704 to transfer therapy data to the electronic device 704.

In some examples, the therapy data comprises dose or dosage data corresponding to one or more doses of medicament provided by the AMP 702 to the subject. Further, the therapy data may comprise subject data corresponding to a medical or physiological state of the subject as determined by the AMP 702 (e.g., using the sensor 618).

In some examples, the data provided to AMP 702 may include any type of data that may be measured or obtained by the AMP 702 and may include a record of therapy provided by the AMP 702. For example, the data may include a time that therapy was provided, an amount of medicament provided as part of the therapy, a measure of one or more vital signs of the subject, a measure of blood glucose levels (e.g., measured blood glucose level) at different times for the subject, a location of the subject, and the like.

In some cases, the electronic device 704 may analyze the therapy data received from the AMP 702 and generate a therapy report. The therapy report may include data relating to the subject's disease, treatment by the AMP 702, anonymized comparisons with other subjects, statistical data relating to the subject's treatment, statistical data relating to other subjects' disease or disease management, and the like. For example, the therapy report may determine whether the subject is maintaining blood glucose levels on average or whether control parameter settings for the AMP 702 are similar to an average subject with similar physiological characteristics as the subject associated with the AMP 702.

In some examples, the data, therapy data, and/or the therapy report may be stored in a memory of the electronic device 704 and/or at a storage of the networked-computing environment.

In some examples, the therapy report or therapy data that is received and/or generated by a first electronic device may be transferred to a second electronic device via a wired or wireless digital data connection. For examples, therapy data or therapy report may be transferred from a cloud server 710 to a mobile phone 708 or a personal computer 706.

In some cases, the first electronic device may be configured to at least receive a request from the second electronic device to access the therapy report, therapy data or other data received by or stored in the first electronic device. In some cases, the second electronic device may be a computing system of a medical practitioner (e.g., such as a doctor, nurse, physician's assistant, etc.), a guardian of the subject (e.g., subject's parents), an authorized user (e.g., a user authorized by the subject such as spouse, relative, friend, and the like), a healthcare provider, or a device of the subject (e.g., mobile phone, personal computer, tablet and the like).

In some examples, the request to access the therapy data, therapy report, or other data may include an account identifier associated with a user that generated the request. In some examples, the account identifier may comprise a unique identifier associated with the subject. Alternatively, or in addition, the account identifier comprises a unique identifier associated with a user that is authorized to access the therapy report. The user may or may not be the subject. In some aspects of the present disclosure, the method may further include associating the therapy data with the account identifier at a storage of the networked-computing environment. Further, the first electronic device may be configured to determine whether an account associated with the account identifier is permitted to view the therapy report. In some examples, account permissions may be granted and/or modified by the subject. For example, the subject can access an account at a networked computing environment, for example, a cloud network provided by a cloud service provider associated with the subject, and provide one or more identifiers associated with one or more other users to give them permission to access the subject's therapy data or report stored on a cloud server 710.

Responsive to determining that the account is permitted to view the therapy report, the first electronic device may transmit the therapy report to the second electronic device over an encrypted communication channel (e.g., created by using asymmetric key pairs to encrypt the data transmitted). Alternatively, or in addition, a shared secret may be determined for the first and the second electronic device. The shared secret may be used to encrypt the therapy report.

An association between a subject, a clinic, and/or an ambulatory medical device may be performed by association of a device serial number of the AMP 702 with the subject and/or clinic. Further, a user (e.g., a subject, clinician, or parent) can access therapeutic recommendations through the cloud in case either the ambulatory medical device (e.g., an insulin pump) or the CGM sensor fails to function.

In some cases, the method may include receiving an identity or identification information of one or more users that are authorized to access therapy data stored at the networked-computing environment. For example, a user or subject may authorize a clinician or other healthcare provider, a parent or guardian, or other users that the subject desires to have access to the therapy data. The identity information of the one or more users may include any type of information that may identify the user or enable the user to be authenticated. For example, the identity information may include a name, unique identifier (e.g., social security number), an email, an address, a phone number, account information for the user at the networked-computing environment, or any other identifying information.

### Managing Control Parameters in an Ambulatory Medical Device

It is often desirable to modify therapy settings of an AMP to improve a health condition of a subject who receives therapy from the AMP. A therapy setting may include values of one or more control parameters that control therapy delivery to a subject. In some AMPs, the control parameters may be changed via therapy change controls provided in a user interface of the AMP. For example, when an ambulatory medicament pump (AMP) controls the blood glucose level in a subject, modifying therapy settings may comprise changing a glucose level set point, a basal dose, a meal dose, a correction does, or type of insulin delivered to the subject. In some cases, a healthcare provider may decide to modify the therapy settings based on the results of medical examinations, blood tests, history of patient's response to therapy provided by the AMP, a combination of these factors or other reasons. In some other cases, the subject or an authorized user of the AMP may desire to modify the therapy settings during a test period in order to evaluate the outcomes of different therapy settings and/or to identify a therapy setting that results in an improvement of the subject's health condition as compared to other settings used during the test period or previously used.

Given the risks associated with selecting incorrect therapy settings by an inexperienced or untrained user, in some embodiments, access to the one or more therapy change controls of an AMP may be limited or restricted. For example, access to certain therapy change controls may be only granted to a user or a subject for a limited time and/or based on an evaluation process that takes into account the user's or the subject's knowledge, experience, level of training, age, mental health or other conditions/factors that may affect the ability of the user or the subject to modify the therapy settings without putting the subject's health at risk or while limiting the risk to the subject. In some examples, access may be only provided to certain therapy change controls required for changing control parameters that are identified based on the health condition of the subject (e.g., determined through medical tests and/or based on therapy data collected during one or more therapy periods). In some examples, the range of values that can be selected for one or more control parameters may be limited to avoid potential harmful effects that may result from setting the value of those parameters outside of the identified “safe range” for a particular subject. In some cases, access to one or more therapy change controls may be provided to a user who may use the AMP for medical research. In yet other cases, access to one or more therapy change controls may be granted to an application developer who is developing, modifying, or debugging a control software used by AMP's control system. In some embodiments, access to a plurality of therapy change controls may be granted for a limited time.

To provide user access to one or more therapy change controls of an AMP without putting the health of a subject at risk, in some embodiments, access to therapy change controls may be managed using a plurality of safe access levels for the AMP. Each of the safe access levels may correspond to one or more control parameters that can be modified by a user or a subject who is qualified to modify the one or more control parameters and/or is associated with the corresponding safe access level. In some examples, access to the selected control parameters may be allowed by enabling access to the corresponding therapy change controls in the AMP (e.g., activating or displaying one or more therapy change control elements on a touchscreen user interface).

In some embodiments, the user may be qualified for a safe access level only during a validity period of the safe access level. After the validity period, the therapy change controls associated with the safe access level may become inactive or unavailable until the user or the subject is revaluated for the same or another safe access level during another validity period. In some embodiments, the AMP may provide the user with the option of extending the validity period of the safe access level. In some embodiments, the validity period may be open ended or may expire when the subject associated with the ambulatory medicament pump changes. In some other embodiments, the validity period can be one hour, one week, one month or other time periods.

In some cases, safe access levels may be categorized based on the capacity of a user for modifying control parameters without causing harm or increasing the risk of harmful effects to the subject. For example, safe access levels may be defined based on age, training, experience, and the like. In other cases, safe access levels may be defined or categorized based on a therapy history of the subject. For example, safe access levels may be defined based on certain events or behaviors identified in the therapy data collected in the previous therapy periods when the subject was receiving therapy from the AMP. In yet other cases, one or more safe access levels may be defined based on a desire to modify therapy settings when the AMP does not provide therapy to human subjects and/or is used to perform investigative research. For example, a safe access level may be dedicated to users who use the AMP for medical research and another safe access level may be dedicated to users who develop applications for the AMP.

In one non-limiting example, four safe access levels (G0 to G3) may be defined for four groups of users where: G0 may be the safe access level for children or certain adults with diminished mental capacity. The therapy change controls available to this group may permit changing the value of one or more control parameters to preset values or back to values previously used. However, other changes to the therapy change controls or changes to other therapy change controls may be restricted. G1 may be a safe access level that is associated with adults who have little experience and/or have only received basic training for using the AMP. The therapy change controls available to this group may enable modification of few control parameters within a safe range. G2 may be a safe access level that is associated with experienced adults and/or have received advanced training. The therapy change controls available to this group may enable modification of several control parameters within a range that may include parameter values that may not be safe to use or that are only safe to use in limited circumstances. G3 may be a safe access level that can be associated with medical researchers or application developers. The therapy change controls available to this group may provide access to a larger number of therapy change controls and allow selecting a wide range of values for each of the control parameters. In some cases, the settings may include values that are unsafe for a typical subject. It should be understood that the aforementioned safe access levels is just one example set of safe access levels. Greater or fewer numbers of safe access levels may exist. Further, the safe access levels may be associated with different types of users and/or may provide different levels of control over the AMP.

For example, the AMP may be associated with three safe access levels (T1 to T3). The three access levels may comprise access levels defined based on the level of training received by a user or the subject who receives therapy from the AMP. T1 may be the safe access level associated with users who have received a basic level of training for the AMP. T2 may be the safe access level associated with users who have received an intermediate level of training for the AMP. T3 may be the safe access level associated with users who have received advanced training for the AMP. The determination of basic, intermediate, or advanced training may be made or specified by a healthcare provider and/or manufacturer of the AMP. In some examples, the therapy change controls available to each of the groups may only provide access to the control parameters that are associated with the requisite training. In some cases, the training may be provided by a healthcare provider, a physician, or a trainer who works under the supervision of the physician or the healthcare provider. In some cases, training data may be used by the healthcare provider or an electronic device to determine the training level for a user or a subject. Training data may comprise, a training certificate (e.g., a digital training certificate), a list of training courses taken or passed, AMP features and control parameters included in the training, and the like.

In some embodiments, new safe access levels may be defined by a manufacturer, healthcare provider, or other users. The new safe access level may be defined based on one or more control parameters of the AMP that are accessible to a user. Further, a new safe access level can be defined by combining existing safe access levels and/or by specifying different criteria to determine whether a user may access a control parameter. For example, three safe access levels may be defined to limit the therapy change controls accessible by different categories of users, such as children, adults who may have different training levels, and/or users who may have limited or reduced mental capacity. In one example, a safe access level may be formed by combining the aforementioned safe access levels G0 and T1, G0 and T2, and G0 and T3. These three new safe access levels may be used to provide access to selected therapy change controls to child users that have received basic, intermediate, or advanced training, respectively.

In some cases, a safe access level may permit selecting specific values of one or more control parameters. In some examples, a safe access level may enable modifying the corresponding control parameters during a set safe access period associated with the safe access level. In some examples, the safe access period for one or more control parameters associated with a safe access level can be different from the safe access period for other control parameters associated with the safe access level.

In some embodiments, a safe access level may be stored in a memory of the AMP as a safe access profile that can include a list of therapy change controls associated with the safe access level. In some examples, a safe access profile may be used by the controller of the AMP (e.g., controller 602 of AMP 702) to enable access to the corresponding therapy change controls upon receiving an indication that the subject receiving therapy from the AMP or an authorized user, is qualified for the safe access level associated with the safe access profile.

In some embodiments, the AMP can be an ambulatory medicament pump (AMP) that controls the blood glucose level in a subject by infusing doses of insulin (or insulin and a counter regulatory hormone such as glucagon) into the subject. In some such embodiments, the AMP may continuously measure the blood glucose level of the subject using a sensor and adjust the infusion time and volume of each medicament dose based at least in part on the measured glucose level. For some such AMPs, some non-limiting example control parameters may include, but are not limited to: 1) dose control parameters for autonomous delivery, such as: glucose level set point, bolus size, basal rate, duration of each therapy, 2) correction factors, 3) carbohydrate ratios, 4) control algorithm parameters such as: predicted CGM window, automated correction factors, Kalman filter parameters, control parameters that account for accumulation of insulin in the subject, learning factors, 5) medicament type such as: U200, U100, ultra-rapid insulin, fast-acting insulin, 6) correction doses, and 7) manual meal doses.

In some cases, a safe access level for an AMP may include access to one or more of the above-mentioned control parameters and/or may include specific safe ranges within which these parameters can be changed by the user or the subject.

In some embodiments, access to one or more therapy change controls of an AMP may be provided to the user or the subject, to facilitate identifying the values of one or more control parameters that may result in an improvement of the glycemic control of the subject. In some such embodiments access to the corresponding therapy change controls may enable the user or the subject to actively participate in the adjustment process (e.g., by systematically modifying the control parameters and carefully monitoring the therapy effects). In other embodiments, access to the corresponding therapy change controls may enable the subject (e.g., a child subject or an inexperienced subject) to revert the newly selected values for one or more control parameters to preset values or previously used values, when experiencing adverse health effects during a test period (e.g., a period used to test the therapy effects of newly selected values).

As mentioned above, in some cases the access to therapy change controls may be provided based on a health condition of the subject. As such, safe access levels for an AMP that controls the glucose level in a subject, may be categorized based on previous glycemic control of the subject by the AMP. For example, in some non-limiting cases, there may be three categories of safe access levels: a first category, H1, may be the safe access level for subjects with high risk of hypoglycemia. This access level may provide access to therapy change controls that can be used to adjust one or more control parameters that control the volume or rate of a counter regulatory hormone (e.g., glucagon), needed to avoid hypoglycemia or to increase the blood sugar level during an episode of hypoglycemia. A second category, H2, may be the safe access level for subjects with moderate risk of hyperglycemia. This access level may provide access to the same therapy change controls as the safe access level H1, however the maximum infusion rate or the maximum volume of the medicament infused during each therapy that can be selected by a user who is qualified for this safe access level, may be limited in order to reduce the probability of hypoglycemia. A third category, H3, may be the safe access level for subjects with high risk of hyperglycemia. This access level may provide access to therapy change controls that can be used to adjust control parameters that control the frequency of therapy and/or the amount of medicament delivered during each therapy (e.g., a therapy control parameter associated with accumulation of insulin in the subject), so that the user or the subject can increase the amount of infused medicament during an episode of hyperglycemia. In some embodiments, safe access levels may be categorized based on other aspects of the glycemic control of a subject by the AMP. It should be understood that the above is just one non-limiting example of safe access levels. It is possible for there to exist more or fewer safe access levels. Further each safe access level may be associated with different risk, therapy change controls, user/subject characteristics, and the like.

In some embodiments, a safe access level may be used to allow a subject or a user eligible for the safe access level to change the value of one or more control parameters beyond a normal range that can be selected by users who are not qualified for the access level. In some examples, a safe access level may be used to allow a user or the subject to enable or disable one or more features of the AMP.

As mentioned above, in some embodiments, a safe access level may be limited to a validity period (e.g., a limited or an open-ended validity period, or a validity period that expires when the subject associated with the ambulatory medicament pump changes) after which the user or the subject loses access to the corresponding therapy change controls. For example, the validity period for safe access level H1 described above may be limited to a period during which the subject is expected to have high level of physical activity (e.g., exercise, hiking, swimming and the like). In some cases, a particular user may be granted access to therapy change controls associated with a safe access level for a longer or shorter period than another user.

### Method of Determining Eligibility for a Safe Access Level

In some cases, an electronic evaluation device, a healthcare provider (e.g., a physician) or a trainer who provides training for an AMP (e.g., a certified trainer who works in a physician or a healthcare provider's office and or in hospital) may determine one or more safe access levels for which a subject receiving therapy from the AMP or a user of the AMP, is eligible for. In some cases, the user may be authorized by a healthcare provider or the subject to make changes to the settings of an AMP that provides therapy to the subject. In some other cases, the electronic evaluation device, a healthcare provider or a trainer may determine the eligibility of the user or the subject for a selected safe access level. The selected safe access level may be selected by the user, the subject, the healthcare provider, or the trainer. In some examples, the electronic evaluation device, the healthcare provider or the trainer may also determine a validity period for the safe access level granted to the user. In some examples, the validity period may be longer or shorter than the safe access period associated with the safe access level. In some such examples, the AMP may use the validity period provide access to the corresponding therapy change controls. In some embodiments, the electronic evaluation device may determine whether the period during which access to one or more therapy change controls is provided is limited by the validity period (e.g., determined by the electronic evaluation device) or the safe access level period associated with the safe access level.

The electronic evaluation device can be: a cloud server, the AMP that provides therapy to the subject, a personal electronic device of the user or the subject (e.g., a desktop or laptop computer, a mobile phone), a personal electronic device of a healthcare provider or a trainer (e.g., a desktop or laptop computer, a mobile phone), an electronic device used by the healthcare provider or the trainer (e.g., a computer in a physician's or a healthcare provider's office, a compute in medical center or a clinic, and the like).

In some cases, a physician or other healthcare provider may determine a safe access level for a user and/or a validity period for the safe access level, based at least in part on evaluation data available or the user.

The evaluation data may comprise information about the user or the subject including but not limited to: age, mental condition, health condition, level of training, medical history, therapy data, therapy report, past history of managing the AMP, previous safe access levels granted, results of one or more medical tests (e.g., blood analysis, urine analysis, and the like), training data. The physician may have access to the evaluation data stored in a non-transitory memory (e.g., memory in a cloud server or an electronic device) or may receive the evaluation data from the AMP or another electronic device (e.g., through a wired or wireless digital data connection). In some examples, the therapy data may have been collected by an AMP that controls the subject's blood glucose concentration. In such examples, therapy data may include measured values of blood glucose concentration in the subject, doses of insulin (or glucagon) infused into the subject, infusion times and the like.

In some examples, a physician or healthcare provider may use the evaluation data to determine one or more safe access levels for which the subject or the user are qualified and select a safe access level and a validity period that allow modifying the therapy change control parameters needed to improve a health condition of the subject via therapy.

In some cases, the physician or healthcare provider may provide access to a combination of therapy change controls that are not associated with a single available safe access level, that are associated with multiple safe access levels, or that are split across multiple safe access levels. For instance, in some cases, a first therapy change control may be associated with a first safe access level and a second therapy change control may be associated with a second safe access level. In such cases embodiments, the physician or healthcare provider may create a customized safe access level to enable a subject or a user access to the combination of therapy change controls. Once created, a customized safe access level may be saved and may be available for future use.

In some cases, a trainer of a user of an AMP may determine a safe access level for the user or determine the eligibility of the user for a selected safe access level (e.g., a safe access level selected by a physician or a healthcare provider or a healthcare provider) based at least in part on the level of training provided to the subject or the user. In some such cases, the trainer may use the evaluation data available for the subject or the user, to determine a safe access level for the user. In some cases, the trainer may also determine a validity period for the safe access level determined by the trainer or the selected safe access level.

In some embodiments, the electronic evaluation device may determine a safe access level for a user based on the evaluation data received from an electronic device. In some other embodiments, the electronic evaluation device may determine the eligibility of the subject or the user for a selected safe access level based on the evaluation data received from an electronic device. In some cases, the electronic evaluating device may determine a validity period for the safe access level (determined by the electronic evaluation device) or the selected safe access level.

The evaluation data may comprise information about the subject including but not limited to: age, mental condition, health condition, level of training, medical history, therapy data, therapy report, past history of managing the AMP, previous safe access levels granted, results of one or more medical tests (e.g., blood analysis, urine analysis, and the like), training data. In some cases, the evaluation data may also include one or more prescriptions of the subject (e.g., provided by a healthcare provider), a recommendation from a healthcare provider and the like.

In some examples, the evaluation data may be stored in a memory of the electronic evaluation device. In some other examples, the electronic evaluation device may receive the evaluation data from an electronic device via a wired or wireless data link. The electronic device may comprise: the AMP, a cloud server, a personal electronic device of a health care provider or a trainer, an electronic device used by a health care provider or a trainer or a personal electronic device of the subject or the user.

In some embodiments, the electronic evaluation device may determine a safe access level for a user in response to receiving a trigger. The trigger may be an access request received from an electronic device (e.g., a cloud server, a personal electronic device of the user, a healthcare provider or a trainer, or an electronic device used by the healthcare provider or the trainer), via a wired or wireless data connection. Upon receiving the access request, the electronic evaluation device may use the evaluation data available for the user to determine a safe access level for which the user may be eligible for. The validity period for the safe level access may be included in the access request or may be determined by the electronic evaluation device.

In some examples, the system may determine the eligibility of the user for a selected safe access level in response to receiving a trigger. In some cases, the trigger may be an eligibility request received from an electronic device (e.g., a cloud server, a personal electronic device of the user, a healthcare provider or a trainer, or an electronic device used by the healthcare provider or the trainer), via a wired or wireless data connection. In some cases, a subject who uses an AMP to control his or her blood glucose level, may send an eligibility request for a safe access level to an electronic evaluation device, in order to gain access to one or more therapy change controls associated with the safe access level. For example, the subject may need access to therapy change controls associated with changing the type of insulin received. The subject may select a safe access level, that includes therapy change controls for changing the insulin type and send an eligibility evaluation request for a safe access level to eth electronic evaluation device. After receiving the eligibility request, the electronic evaluation device may search for a prescription for a new type of insulin. If the prescription is found, the electronic evaluation device may search for other types of evaluation data (e.g., training level, age, prior safe access level granted, physician recommendation and the like), to further evaluate the eligibility of the subject for the safe access level.

In some implementations, the electronic evaluation device may determine the eligibility of the user (or the subject) for a selected or requested safe access level using an interactive evaluation process. The interactive evaluation process may include an interaction with the user or the subject via one or more user interfaces of the electronic evaluation device (e.g., a touchscreen display, a keyboard, a monitor, a mouse, an interactive software interface, an interactive web page and the like). For example, the interactive evaluation process may include displaying a set of queries, corresponding to one or more use cases of the AMP 702, to the user and the receiving a set of responses to the set of queries. Subsequently, the electronic evaluation device may evaluate the set of responses to obtain an evaluation score and use the evaluation score to determine selected safe access level for the user.

### Providing Access Based on a Safe Access Level

As described above, the access to one or more therapy change controls of an AMP may be enabled only if a safe access level that is associated with the one or more therapy change controls is granted to a user of the AMP or a subject receiving therapy are from the AMP. In some embodiments, once the safe access level is granted to the user or the subject (e.g., based on an evaluation process performed by a trainer, physician or an electronic evaluation device), the AMP may enable access to one or more therapy change controls upon receiving a passcode (e.g., a time-based passcode) via a user interface (e.g., a passcode entry interface), or receiving an access signal (e.g., from an electronic access device). The access signal can be an analog or a digital signal. In some cases, the information regarding the safe access level granted to the subject may be encoded in the access signal. For example, the granted safe access level signal, one or more therapy change controls associated with the grated safe access level, and/or a validity period of the granted safe access level may be encoded in eth access signal.

In some embodiments, the access to one or more therapy change controls of an AMP may be passcode protected. In some such embodiments, when a safe access level and a validity period are determined for a subject who receives therapy from an AMP (or an authorized user of the AMP), an electronic access device or the electronic evaluation device may generate and send a passcode (e.g., a time-based passcode), associated with the safe access level and the validity period, to the AMP. In some examples, the time-based passcode and the validity period are stored in a memory of the AMP and may be used to provide access to the therapy change controls corresponding the safe access level during the validity period. In some such examples, the time-based passcode may expire after the validity period.

In some other embodiments, when a safe access level is determined for a subject who receives therapy from an AMP (or an authorized user of the AMP), an electronic configuration device may generate a passcode (e.g., a time-based passcode), associated with the safe access level based at least in part on a public identifier received from the AMP. In some such embodiments, the passcode may be provided directly to the user or the subject by an operator or administrator of the electronic configuration device over a communication link (e.g., a wired or wireless phone line). In some examples, the operator or the administrator may provide the passcode via a voice call, voice message, or text message. In some other examples, the electronic configuration device may send the passcode via a text message to the user's phone (e.g., a smart phone).

In some cases, where a healthcare provider or a trainer determines the eligibility of the subject or the user for a safe access level, they may use an electronic access device to generate the time-based passcode and send it to the AMP. An electronic access device can be a cloud server, a personal electronic device of a healthcare provider or a trainer (e.g., a desktop, laptop computer, or a mobile phone), or an electronic device used by the healthcare provider or the trainer (e.g., a computer in a physician's or healthcare provider's office, a compute in medical center or a clinic, and the like). For example, a physician or a healthcare provider may select the safe access level and send the corresponding time-based passcode to the AMP using a user interface of the electronic access device.

In some embodiments the electronic evaluation device that has determined the safe access level and the validity period for the user or has determined the eligibility of the user for a selected safe access level, may generate the corresponding time-based passcode and automatically transmit it to the AMP using a wired or wireless data connection.

When a time-based passcode associated with a safe access level is received and stored in a memory of the AMP, the controller of the AMP may provide a passcode entry element on a user interface of the AMP where the user or the subject can provide a passcode by interacting with the user interface. In some examples, the AMP may also receive (e.g., from the electronic evaluation or access device) a validity period for the safe access level. In such examples, the passcode may expire after the validity period.

In some embodiments, the access to one or more therapy change controls of an AMP may be enabled using an access signal. In some such embodiments, when the a safe access level and a validity period are determined for a subject who receives therapy from an AMP (or an authorized user of the AMP), an electronic access device or the electronic evaluation device may generate and send an access signal, associated with the safe access level and the validity period, to the AMP. In these examples, upon receiving the access signal, the AMP may provide user access to one or more therapy change controls associated with the safe access level during the validity period.

In some embodiments, the therapy change controls associated with a safe access level may be stored in a memory of the AMP as a safe access profile. In some examples, the AMP may store a plurality of safe access profiles associated with a plurality of safe access levels (e.g., safe access-levels G0-G3 or T1-T3 described above). A safe access profile may comprise a list of therapy change controls for changing control parameters associated with the corresponding safe access level. Alternatively, or in addition, the safe access profile may include a range of values that may be selected for the control parameters. In some examples, the safe access profile may comprise a safe access period for the safe access level. In these examples, the access to the corresponding therapy change controls may be denied after the safe access period. In some example, a validity period received with the safe access level, may reduce or extend the safe access period for a safe access level.

In some embodiments, instead of generating and sending a passcode, the electronic access device or the electronic evaluation device may send an access signal to the AMP to enable access to the therapy change controls associated with a safe access level granted to a user of the AMP. The access signal may be transmitted from the electronic access device or the electronic evaluation device via a wireless data connection (e.g., via a LAN, WAN, Bluetooth, or an NFC signal). In some examples, the wireless data connection may be a direct end-to-end connection. When the access signal is received by the AMP, the AMP may provide access to the therapy change controls included in the safe access profile associated with the safe access level. In some examples, the access signal received by the AMP may enable access to the therapy change controls, associated with the granted safe access level during a validity period determined by the electronic evaluation device, a healthcare provider or a trainer.

In some examples once a safe access level is determined, it may be stored in the electronic evaluation device, electronic access device, the AMP or other electronic devices (e.g., an electronic device of the user, the subject, the healthcare provider, or the trainer). In some examples, one or more determined safe access levels that have been stored, may be used to provide access to the corresponding therapy change controls on a new AMP. In some examples, one or more determined safe access levels that have been stored, may be used to extend the safe access period or determining eligibility for the same and or another safe access level at a later time.

In some embodiments, an authorized user or a subject who has been granted a safe access level can change one or more of the corresponding control parameters to create a personalized therapy setting. A personalized therapy setting may be used for an unlimited time period or set indefinitely, expire after a set time period (as may be set, for example, by a physician), expire at the end of the safe access period (e.g., when time-based password expires), or expire after use or modification of a therapy change control (either once or a set number of times). In some cases, the user or the subject may change the personalized setting to a standard setting (e.g., shared setting) at any time or before a set period. In some cases, the personalized therapy setting may be used until a trigger occurs or until a new therapy setting is received.

In some examples, if the AMP detects that an unsafe level of therapy is delivered or may be delivered as a result of changes made based on a safe access level, the AMP may automatically change the value of the corresponding control parameters to safe values or may reject a particular change to a therapy change control. Safe values may be previously used values, values determined by the AMP based on past or present therapy data, or other values stored in a memory of the AMP. For example, if the blood glucose level of a subject who receives therapy from an AMP that provides insulin to the subject drops below a threshold value as a result of reducing the glucose set point during a safe access period, the controller of the AMP may automatically change the glucose set point back to an earlier set value.

In some embodiments, the validity period of a safe access level granted to a user or a subject receiving therapy form the AMP, may be extended by the user or the subject. For example, the user or the subject may generate a request for extension of the validity period via a user interface of the AMP. In some cases, the request for extension may be transmitted to an electronic evaluation device that determines whether the requested extension can be granted the user or the subject. Upon determining that the extension can be granted, the electronic evaluation device may send an extension signal to the AMP. Upon receiving the extension signal AMP may extend the expiration of the corresponding time-based passcode to a new ending time or enable access to the corresponding therapy change controls until the new ending time. In some examples, the ending time may be included in the request for extension. In some other examples, the ending time may be determined by the electronic evaluation device. In some embodiments, the user may send the request for extension to a healthcare provide or a trainer. In some such embodiments, the request for extension may be transmitted from the AMP to an electronic device of the trainer or the healthcare provider, directly or via an intermediary electronic device. The healthcare provider or the trainer, may approve the new ending time included in the request for extension or determine another ending time. The healthcare provider or the trainer, may use an electronic access device to extend the validity period of a granted safe access level.

In some case, after changing the values of one or more control parameters associated with a granted safe access level, a user or a subject may desire to restore previously used values of the one or more control parameters. In some such cases, the AMP may provide the user or the subject with the option of restoring previously used values of the one or more control parameters. For example, the AMP may provide a user interface (e.g., a menu on a touchscreen interface) that includes one or more restoring elements that may enable restoring the previously used values of the one or more control parameters.

As mentioned above, the validity period of a safe access level granted to a user (or the subject receiving therapy from the AMP 702), may be extended by the user or the subject. In some implementations, extending the validity period may require re-evaluating the eligibility of the user for the safe access level that was previously granted to the user based on an initial evaluation process described above. In some cases, unlike the initial evaluation process, the re-evaluation process may not involve the physician, the healthcare provider or the trainer. For example, the subject may send a validity period extension request to the electronic evaluation device (e.g., via a wired or wireless connection, or a user interface), and the electronic evaluation device may re-evaluate the eligibility of the user for the safe access level based on a record of the user's use of the pump during the validity period. For example, the electronic evaluation device may determine whether changes made by the user to the one or more therapy control parameters resulted in increased occurrences or risk of hypoglycemia or hyperglycemia during the validity period. If the electronic evaluation device determines that user's use of the pump during the validity period did not result in increased occurrences or risk of hypoglycemia or hyperglycemia during the validity period, it may extend the validity period of the safe access level and allow the user to use the corresponding control settings of the AMP during an extended validity period. In some cases, the electronic evaluation device may also use the evaluation data, which may have been updated during the validity period, to re-evaluate the eligibility of the user for the safe access level. In some cases, the electronic re-evaluation device may use the interactive evaluation process described above to re-evaluate user's eligibility for the safe access level for an extended validity period. In some cases, if the electronic evaluation device determines that user's use of the pump during the validity period resulted in periods of hypoglycemia or hyperglycemia during the validity period, it may deny the validity period extension request and notify the user (e.g., via a user interface of the AMP 702 or a user interface of the electronic evaluation device).

### Additional Features

In some embodiments, the settings (e.g., therapy settings) of an AMP may be changed remotely using an electronic control device. For example, an electronic control device may transmit a command to the AMP via a wired or wireless digital data link to change the values of one or more control parameters of the AMP or change certain settings of the AMP. The electronic control device can be personal computer, a mobile phone a cloud server or other computing devices configured to communicate with the AMP. In some examples, the data connection between the electronic control device and the AMP may be established using one of the methods described with respect to FIG. 7.

In some embodiments, when a user or a subject obtains a new AMP, a onetime passcode may be provided to the user or the subject that can be used to download the therapy settings and subject's data from a remote electronic device, in order to activate and update the new AMP. In some such embodiments, if the new device is activated and updated during the validity period of a safe access level (previously granted to the user or the subject), the AMP may allow the user or the subject to access the corresponding therapy change controls using the previous passcode associated with the safe access level. In some examples, if the new AMP is activated and updated during the validity period of a safe access level, the AMP may automatically enable access to the corresponding therapy change controls without requesting a passcode.

### Preventing Inadvertent Therapy Changes

As described above, the ambulatory medicament device may include a user interface (e.g., touchscreen interface or a non-touchscreen interface) that may present one or more user-interface screens to a user enabling the user to modify one or more therapy settings of the ambulatory medicament device, such as a quantity of medicament delivered when a condition is met or the condition that triggers the delivery of medicament to a subject. The user may be a subject receiving medicament or therapy, or may be another user, such as a clinician or healthcare provider, or a parent or guardian. For ambulatory medicament devices that include a user interface, there is a risk that a setting is accidentally modified or is modified (intentionally or unintentionally) by a user that does not fully comprehend his or her action (e.g., a child or a user with a reduced mental capacity). Further, ambulatory medicament devices may accidentally have settings modified by inadvertent interactions with a user interface, such as may occur when an ambulatory medicament device is worn against the body of a subject.

This section relates to an ambulatory medicament device (AMD) to prevent an inadvertent modification in medicament deliver, for example, in the event of a setting of the AMD being accidentally modified by a user or inadvertent interactions with a user interface.

As mention above, in some embodiments the user may modify the control or configuration the AMD using a user interface. There is a possibility that the control or configuration of the AMD is accidentally modified through the user interface. For example, as the user may transport the ambulatory medical device, there is a danger that the user will inadvertently activate input in the ambulatory medical device that initiates a therapy change input (e.g., by applying pressure on the ambulatory medical device that may be placed in the jacket pocket of the user).

With reference to FIG. 8, in some such embodiments, the control and computing module (CCM) of the AMD may include a set of therapy change procedures 818 implemented to prevent therapy change inputs 820 that are inadvertent. The therapy change procedures 818 may be implemented as instructions stored in a memory of CCM and executed by the processor. The therapy change input 820, received from a user 816, may be verified by the therapy change procedures 818 before the ambulatory medical device provides the therapy change delivery 804. All the user interactions with the user interface module 806 may be controlled and analyzed by the control and computing module (CCM) via one or more therapy change procedures 818.

In these embodiments, the user 816 may wake or unlock the AMD by interacting with a wake interface 810. The wake interface 810 can be any of the additional user interfaces mentioned above, configured to generate a wake input to the CCM when detecting a pre-set user interaction.

The therapy change input 820 can be an input provided by the user 816 to change a therapy that is currently being delivered to the user 816. In some embodiments, the therapy change input 820 may cause the insulin or glucagon infusion pump to start infusing an amount of insulin or glucagon into the user 816. Alternatively, the therapy change input 820 may modify the rate of insulin or glucagon infusion into the user 816. The therapy change input 820 may also cancel insulin or glucagon infusion into the user 816 from the insulin or glucagon infusion pump.

When a wake action is detected by the wake interface 810, a wake input is sent to the control and computing module wherein it imitates a wake control procedure 826 that generates a wake signal to wake/unlock the user interface (e.g., a touch screen display).

When in the wake and/or unlocked state, a user may interact with the touchscreen display 812, alphanumeric pad 814 or other types of user interfaces that may be included in the user interface module 806, to obtain access to therapy change user interface.

The therapy change user interface may be activated by a first user interaction with the user interface (e.g., touchscreen display 812). When the first user interaction is detected, the user interface module user interface module 806 sends an input signal to the control and computing module wherein it is analyzed by a therapy change control procedure 828. If it is determined that the first user interaction satisfies a set of predefined conditions, the therapy change control procedure 828 generates a signal to the user interface module user interface module 806 to activate the therapy change user interface.

In some embodiments, the therapy change user interface may be limited based on the first user interaction. For example, the therapy change control procedure 828 may send one of two signals to the user interface module user interface module 806. The therapy change user interface may then unlock one of two different therapy change user interfaces that result in different options of therapy change selections for the user 816. In an implementation of this example, a therapy change selection to make a significant therapy change, such as dramatically increase the rate of insulin or glucagon infusion rate, requires a first user interaction that is different from the first user interaction that would be required for an insulin or glucagon infusion at a normal or prescribed rate. In some examples, the first user interaction may be a simple interaction (e.g., a simple gesture) that unlocks a therapy change user interface with therapy change selections that are limited. Another first user interaction may be a complicated interaction (e.g., a series of complex gestures) that unlocks a therapy change user interface with therapy change selections that have no limits. An example of this implementation may be useful for child users. The child user may perform the first gesture that is made up of a series of simple inputs to unlock therapy change selections that are limited. An adult user may perform the first gesture that is made up of a series of complex inputs to unlock the therapy change user interface with therapy change selections that have no limits.

Once activated, the therapy change user interface that may provide one or more control or configuration elements that enable the user to modify the control or configuration of the ambulatory medicament device. The control or configuration element may include any type of user interface screen on the touchscreen, or other type of user interface in the non-touchscreen context, that enables or permits a user to change a configuration of the ambulatory medicament device. This change in configuration of the ambulatory medicament device may relate to a change in the therapy provided or in the detection of a triggering event that causes therapy (e.g., medicament) to be provided to a subject. For example, the change in configuration may include a selection between one or more hormones that regulate blood sugar level (e.g., insulin or glucagon) of a user, an amount of the one or more hormones that regulate blood sugar level of the user.

In some cases, a change to the configuration of the ambulatory medicament device is automatically and/or instantly recognized or implemented by the ambulatory medicament device, and/or transmitted to the ambulatory medicament device. In other cases, a confirmation of the change may be required before the change is implemented by or transmitted to the ambulatory medicament device.

This confirmation may be entered based on a second user interaction with a user interface (e.g., touchscreen display 812). When the second user interaction is detected, the user interface module user interface module 806 sends an input signal to the control and computing module wherein it is analyzed by a therapy change control procedure 828. If it is determined that the second user interaction satisfies a set of predefined conditions, the therapy change control procedure 828 implements the change to the configuration of the AMD.

The first and/or second user interactions may include the selection of an icon, a series of taps or inputs, one or more gestures (e.g., a linear swipe, an arcuate swipe, a circular swipe, or other simple or complex movement across the touchscreen), performing a pattern or sequence on the touchscreen (e.g., drawing an image), a multi-touch or multi-input interaction, a combination of the foregoing, or any other type of interaction with a touchscreen, or portion thereof. The series of inputs may be any combination of touch movements, touch points, numerical characters, alphabetical characters, and other symbols. Gesture interactions can be guided by visual indicia displayed or printed on the AMD. In some embodiments, the visual indicia can include animations that suggest or guide user interactions with a touchscreen. For example, the first user interaction can include an arcuate swipe around at least a portion of a generally circular icon or logo. In some examples, the first and/or second user interactions may include a predetermined sequence of numerical or alphabetical inputs. In some examples, a series of multiple inputs, the range of parameters for an input may be dependent on other inputs in the series. For example, required start position of a touch movement may be dependent on the position of the previous touch movement. The time that the series of inputs are entered may also be a part of the range of parameters. For example, a series of inputs may need to be entered in no less than 3 seconds or more than 3 seconds, and no more than 15 seconds or less than 15 seconds.

Further, one or more of the interactions may include interacting with a sensor as an optical sensor (e.g., visible light or IR sensor), biometric sensor (e.g., a fingerprint or retinal scanner), a proximity sensor, a gyroscope, or a combination of accelerometer and gyroscope, and the like. Also, in an exemplary embodiment, the second user interaction may be made through a wireless signal such as RFID or Bluetooth. In some embodiments, the second user interaction may include receiving a selection of an indicator box that correspond to either insulin or glucagon and receiving a predetermined sequence of numerical inputs in order to deliver the therapy change selection.

The type of user interaction that unlocks the touchscreen, provides access to a configuration screen, and/or confirms a change to the configuration of the ambulatory medicament device may be the same or may differ.

In an exemplary embodiment, the system may have a time-out such that if no interaction occurs for a set period of time, the user interface will turn off and the therapy change request process has to start again. In one implementation of the time-out, if no interaction occurs for more than 30 seconds after the system is waked/unlocked before the second user interaction is received by the user interface, the user interface will be deactivated.

Once the configuration change is confirmed, implemented, or transmitted, the ambulatory medicament device may begin operating with the changed configuration.

This operation may include triggering therapy based on the new configuration or providing therapy based on the new configuration. For example, the ambulatory medicament device may generate a dose control signal based at least in part on the modified configuration or control parameter, or may detect a trigger based at least in part on the modified configuration or control parameter that leads to the provisioning of therapy.

With reference to FIG. 8, in some embodiments, the changes made through the therapy change user interface are sent to CCM wherein the therapy change control procedure 828 in CCM transfers the changes to the device and subject monitoring procedure 824. The device and subject monitoring procedure 824 may be implemented in the CCM to monitor the status of the AMD (e.g., therapy delivery configuration) and the health condition of the user 816 (or a subject). For example, the subject monitoring procedure 824 may receive information about a therapy change requested by a user 816 through a user interface (a touchscreen display 812 or alphanumeric pad 814) or information about glucose level in subject's blood from the subject sensor 808. Subsequently, the device and subject monitoring procedure 824 may transmit the information pertaining a health condition of the subject and/or the AMD configuration, to the medicament dose control procedure 822. In some examples, the parameters in the medicament dose control procedure 822 may be adjusted based on the changes and/or information captured by the device and subject monitoring procedure 824. The medicament dose control procedure 822, controls the medicament delivery interface 802 by providing a medicament dose signal. The medicament does control may be generated based on detected conditions or physiological characteristics of the subject (e.g., provided by the readings of the subject sensor 808) and according to parameter values received from the therapy change control procedure 828. The medicament delivery interface 802 may provide a therapy change delivery to the user according to the information received by device and subject monitoring procedure 824.

In some examples, the dose control signals may be produced based on time (e.g., medicament may be delivered on a periodic basis), one or more a command, indication that the subject is planning to engage or is engaging in a particular activity (e.g., eating a meal, exercising, sleeping, fasting, etc.), or any other factor that may relate to or cause the triggering of therapy (e.g., medicament delivery).

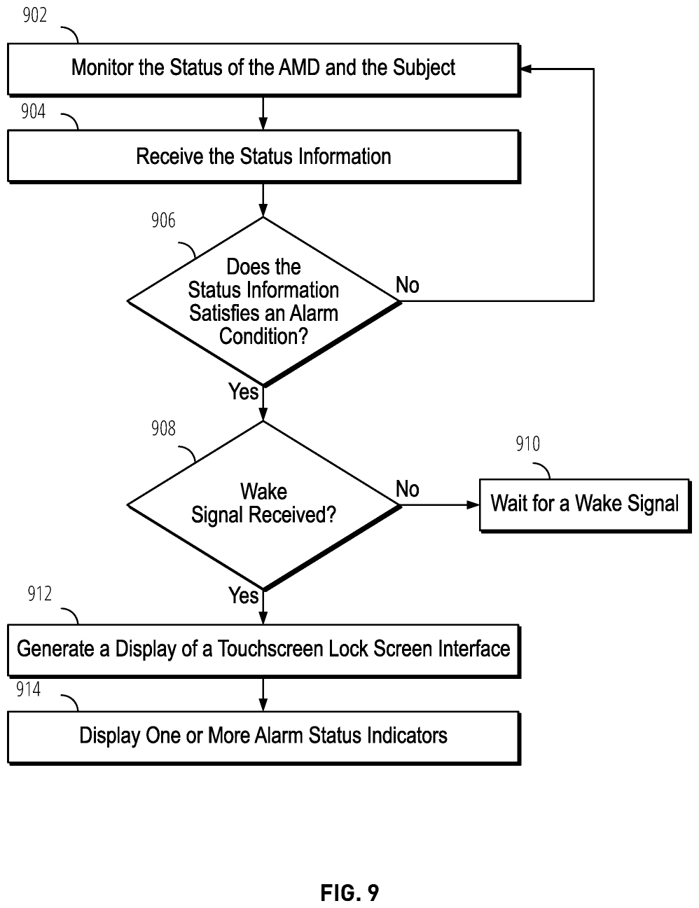

FIG. 9 is a flow diagram illustrating an example method that may be used by an AMD to generate an alarm status indicator. In some embodiments the device and subject monitoring procedure (excused within CCM), may continuously monitor the status of the AMD (e.g., the user interface, different modules of the AMD and the like) as well as the health condition of a subject (e.g., using various subject sensors such as analyte sensors) 902. Once a status information is received 904, the device and subject monitoring procedure may determine whether the received status information satisfies an alarm condition 906. If the received status information does not satisfy an alarm condition, no cation will be taken and device and subject monitoring procedure continuous monitoring the AMD and the subject. If it is determined that the received status information satisfies an alarm condition, the system search for a wake signal 908. If no wake signal is detected, the systems waits for a wake signal to be received 910. Once a wake signal is received via one or more user interfaces or sensors, the CCM may generate a display of a touchscreen lock screen interface 912 and display one or more alarm status indicators 914, corresponding to the detected alarm condition, on the lock screen.

AMD with Alarm System

In some cases, a condition may occur that impacts the operation of the ambulatory medicament device. This condition may be associated with the ability of the ambulatory medicament device to operate as intended by the manufacturer, a subject receiving therapy from the ambulatory medicament device, and/or user (e.g., healthcare provider, parent, or guardian of the subject). In some cases, the ambulatory medicament device may be operating as intended, but the condition of the subject may not satisfy a desired level of health. In either case, it is generally desirable to generate an alarm to inform the subject and/or one or more users of the condition of the ambulatory medicament device and/or the subject. Moreover, it is desirable to track the alarm until the condition that caused the alarm is resolved. Further, it is desirable to issue different types of alarms for different conditions to enable a subject or user to easily distinguish the severity of the condition that triggered the alarm. The user may be a subject receiving medicament or therapy, or may be another user, such as a clinician or healthcare provider, or a parent or guardian.

This section of the disclosure relates to an ambulatory medicament device, such as an insulin pump or a combined insulin and counter-regulatory agent (e.g., Glucagon) pump, configured to generate a dose control signal configured to cause a medicament pump to infuse medicament into a subject. Moreover, the present disclosure relates to an ambulatory medicament device configured to detect a condition of the ambulatory medicament device and/or the subject, and to generate an alarm when it is determined that the detected condition satisfies an alarm condition.

As mention above, an ambulatory medicament device may include an alarm system configured to monitor the ambulatory medicament device and/or the subject, and to generate an alarm when it is determined that a condition has been detected that satisfies an alarm condition. In some examples, the alarm system that may organize a list of alarms, notifying a user of these alarms, and allowing the user to acknowledge alarms.

In some embodiments, the alarm system may comprise a plurality of sensors that monitor the AMD or the subject, a monitoring system interface that receives the data from sensors, and alarm generation module that process the received data and generate alarms if an alarm condition is met. In some examples, the monitoring system interface and the alarm generation module are implemented using one or more hardware processors and machine readable. In some other examples, the monitoring system interface and the alarm generation module are separate hard ware modules.

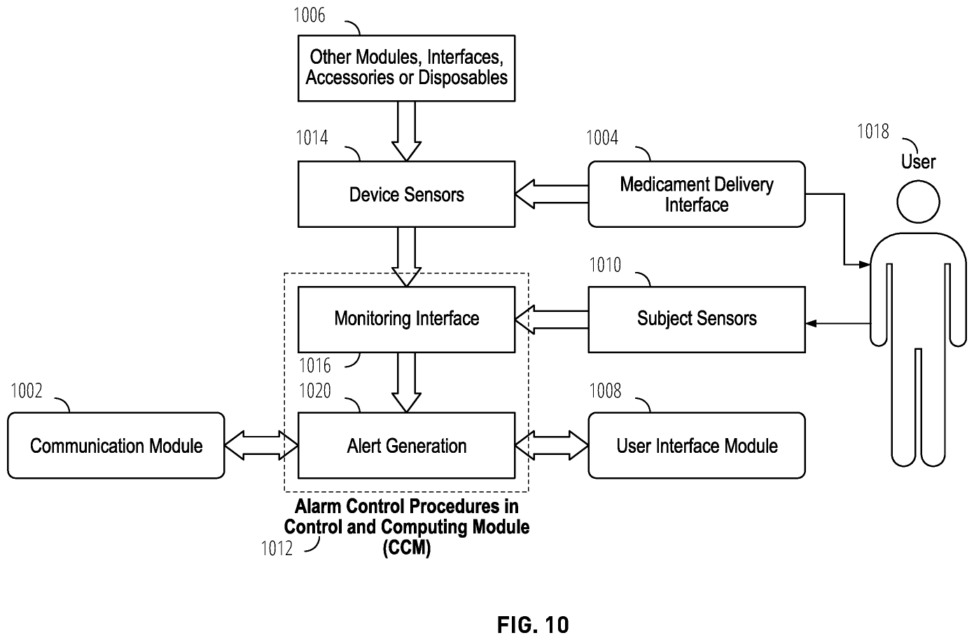

With reference to FIG. 10, in some embodiments, an alarm control procedure 1012 implements alarm control procedures in the control and computing module (CCM) of the AMD. The alarm control procedure 1012 can be implemented as instructions stored in a memory of the CCM and executed by a hardware processor to generate an alarm upon detection of a condition of the ambulatory medicament device and/or the subject. In some cases, the hardware processor of the monitoring system is a hardware processor of the ambulatory medicament device that controls medicament delivery. In other cases, the hardware processor of the monitoring system may be a separate hardware processor.

In some examples, the alarm control procedure 1012 includes a monitoring interface 1016 and an alert generation 1020 system. The monitoring interface 1016 monitors the condition or status of the AMD and/or the subject at least partially based on signals or status values received form a set of device sensors 1014 and a set of subject sensors 1010. In some examples, the device sensors may be configured to track the status of the components or the elements of the ambulatory medicament device, and the subject sensors can be configured to obtain measurements of one or more physiological characteristics of the subject

In some examples, a device sensor 1014 is a sensor that generates a signal or status value associated with the condition of modules, interfaces, accessories, other modules, interfaces, accessories, or disposables 1006 of the AMD. In some examples, a device sensor 1014 may generate a signal that corresponds to a parameter associated with a component in a module or interface. For example, one device sensor may record the voltage of a battery and another device sensor may record the follow rate of a pump the medicament delivery interface 1004.

In some examples, a subject sensor 1010 may be any sensor that generates a signal or status value associated with one or more physiological indicators (or parameters) of a subject (e.g., heart rate, blood pressure, body temperature, level of blood sugar, serum levels of various hormones or other analytes). The device and subject monitoring interface 1016 can include continuously receiving and analyzing signals received from device sensors 1014 and subject sensors 1010 to determine the condition of the ambulatory medicament device, the subject, a sensor, and/or other accessories.

In some cases, a single sensor may be used to monitor both the condition of the subject and the ambulatory medicament device or accessories and sensors connected to AMD. For example, a continuous glucose monitoring CGM sensor may be used to monitor the condition of the subject, and may also be monitored to determine whether the condition of the CGM satisfies an alarm condition (e.g., to alarm a user that the CGM should be replaced).

Although described as sensors of the ambulatory medicament device, one or more of the sensors may be accessories that may or may not be part of the ambulatory medicament device, but that may communicate with the ambulatory medicament device.

In some examples, alarm control procedure 1012 implements procedures for allowing a user 1018 or the subject to change the alarm settings and/or acknowledging an alarm annunciation via the user interface module 1008. In some examples, the user 1018 may be able to see one or more alarms annunciated on a user interface (e.g., as a list of alarms), even if the AMD is in locked state. In these examples, the user may not be able to acknowledge or respond to alarm when the AMD is in locked state.

In some such examples, a user 1018 or the subject may get access to an alarm setting screen or acknowledge an alarm annunciation by providing a wake signal and a first gesture (e.g., on a touchscreen display). In some cases, the first gesture may be created by entering predetermined characters on the alphanumeric pad. In some such examples, the alarm control procedure 1012 distinguishes inadvertent alarm control inputs from intentional alarm control inputs. An inadvertent alarm control input is an alarm acknowledgment input that was made without the intent of the user 1018 to acknowledge the alarm that the ambulatory medical device is delivering to the user 1018.

In some examples, the alarm control procedure 1012 implements processes for determination and categorization of an alarm condition based on its severity level (e.g., a severity level between 0 and 5), according to the information received through the monitoring interface 1016.

In some other examples, the alarm control procedure 1012 implements procedures for controlling the annunciation of alarm conditions via the user interface module 1008, at least partially, based on their severity level. In some such examples, a user interface (e.g., a touchscreen display) may be configured to allow the user 1018 to navigate directly to the issue or fault for which an alarm is being delivered. This capability provides the user 1018 with access to address the fault causing the alarm so that it could be corrected thereby stopping the alarm.

### Alarm Conditions

In some examples, the device and subject monitoring interface 1016 may provide the status information received from the device sensor 1014 and/or subject sensors 1010 to the alert generation 1020 system. In some examples, the status information may comprise one or more status values. In some such examples, the alert generation 1020 system is configured to determine based at least in part on the status information received from the subject monitoring interface 1016, whether an alarm condition is satisfied.

Determining whether the alarm condition is satisfied may include comparing one or more status values associated with the ambulatory medicament device and/or the subject to one or more alarm thresholds or alarm conditions. In some cases, each alarm threshold or alarm condition may be associated with an alarm profile. In some such cases, determining whether the alarm condition is satisfied may include comparing the status information to one or more alarm thresholds or alarm conditions included in one or more alarm profiles. In some examples, the alarm profile may be stored in a storage of the control and computing module. In some such examples, at least some of the alarm profiles may be provided to the CCM by an authorized user or the subject via a user interface or directly transferred from another device to the storage (e.g., from USB drive, a laptop, smart phone, PC and the like). In some other examples, at least some of the alarm profiles may be stored in the storage at the time of manufacture,

Each of the alarm profiles may indicate the characteristics or status of the ambulatory medicament device and/or subject that triggers an alarm corresponding to the alarm profile. For example, at least some alarm profiles may indicate the threshold status values below of above which an alarm should be triggered. For example, one alarm profile may indicate that when a blood glucose level of the subject exceeds a particular threshold, a particular alarm is to be generated and/or annunciated. As another example, an alarm profile may indicate that when an available amount of medicament is below a particular threshold, a particular alarm is to be generated and/or annunciated. The type of alarm and/or the alarm frequency or intensity associated with the medicament level may differ from the alarm triggered based on the blood glucose level. Although the previous examples, described a single condition associated with a single alarm profile, it should be understood that multiple conditions may be associated with an alarm profile. For example, a blood glucose level that exceeds an upper threshold or is below a lower threshold may be associated with different alarm profiles or the same alarm profile. As another example, a blood glucose level that is above an upper threshold or a medicament pump that is unable to supply insulin may be associated with the same alarm profile. On the other hand, a medicament pump that is unable to supply insulin due to an empty insulin cartridge may be associated with a different alarm profile than if the medicament pump is unable to supply insulin due to damage to the medicament pump.

Some non-limiting examples of conditions of the ambulatory medicament device or of the subject that may be associated with an alarm profile include conditions relating to a battery capacity (e.g., below a threshold charge capacity or below a capacity associated with a particular amount of operating time (e.g., one day)), a battery condition (e.g., high temperature or low voltage), a medicament or drug delivery condition (e.g., medicament is empty or below a threshold, motor is stalled, catheter is occluded, etc.), subject sensor condition (e.g., blood glucose sensor is expiring, or signal was not received from sensor), calibration failure, high or low glucose levels, network (e.g., Bluetooth® or BN-LTE) communication errors, haptic interface errors (e.g., motor non-responsive), speaker errors (e.g., noise or low volume), medicament cartridge errors (e.g., empty cartridge, cartridge detection error, etc.), and the like. As explained below, each of these errors or conditions may be associated with different severity levels that cause the annunciation of different alarms.

In some cases, each alarm profile may be associated with a severity level of the alarm. The severity level may be associated with how urgently the condition that triggered the alarm should be addressed or resolved. Further, the severity level may be associated with an amount of harm that may be caused to a subject if the condition that triggered the alarm is not resolved or is not resolved within a particular time period. The number of severity levels may vary based on the type of ambulatory medicament device. Generally, there is no limit to the number of severity levels. However, there may be a point of diminishing returns as the number of severity levels exceeds a particular number because, for example, it may be difficult for a user to distinguish between the different numbers of severity levels or to identify with which severity level a particular alarm is associated. Thus, the number of severity levels may be limited to a particular number, such as 3, 5, 6, 9, or some number in between. However, it is possible for there to be more than 9 severity levels.

There may be multiple alarm profiles associated with the severity level. Or each condition of the ambulatory medicament device and/or subject that is associated with the same severity level may be associated with the same alarm profile.

The ambulatory medicament device may determine a severity of an alarm condition based on the condition of the ambulatory medicament device and/or the subject that triggered the alarm condition. In some cases, the ambulatory medicament device may determine the severity of the alarm condition based at least in part on an alarm profile associated with the alarm condition.

Generally, if the alarm condition does not prevent the ambulatory medicament device from providing therapy, the ambulatory medicament device may continue to provide therapy. However, if the alarm condition interferes with the delivery of therapy, operation of the ambulatory medicament device may be suspended or partially suspended. Generally, alarm conditions that interfere with the provisioning of therapy may be associated with a higher severity level. However, some alarm conditions that interfere with the provisioning of therapy may be associated with lower severity levels. For example, a determination that the ambulatory medicament device cannot supply insulin may normally be associated with a highest severity alarm. But if a user indicates that the site location is currently in process of being changed, the alarm condition may be associated with a lower severity level (e.g., an informational alarm reminding the user that insulin cannot be delivered during site change).

### Alarm Annunciation

The alert generation 1020 system can implement an annunciation pattern selected based at least in part on the status information generated by and received from the monitoring interface 1016, whether an alarm condition is satisfied. Determining whether the alarm condition is satisfied may include comparing one or more status values associated with the ambulatory medicament device and/or the subject to one or more alarm thresholds or alarm conditions associated with an alarm profile.

Upon verifying that an alarm condition associate with an alarm profile is satisfied, the alert generation 1020 system annunciates the alarm condition.

In some examples, the alarm system may generate a list of pending alarm conditions and store it in a memory of the AMD. In these examples, any time an alarm condition associated with an alarm profile is satisfied, the alarm system may update the list of pending alarm condition by adding the new alarm condition to the list of pending alarm conditions.

In some examples, the list of pending alarm conditions may be sorted according to the severity level associated with the alarm conditions.

In some examples, the alarm system may annunciate the alarm conditions via the user interface module 1008 of the ambulatory medical device 1600. For example, the alarm condition may be annunciated via one or more user interfaces (e.g., a display, a speaker, and the like). In some such examples, an alarm may comprise an audio alarm, a text message, a graphical message, a text or graphical message with audio, vibrations, flashing light and any combination of these.

In some other examples, the alarm conditions may be transmitted to other devices, via the communication module 1002 of the AMD where, for example, an authorized user 1018 (e.g., guardians or parents of the subject), the subject or an emergency provider can view the alarm condition. In yet other examples, the alert generation 1020 system, may establish a direct end-to-end connection with a computing system (e.g., a cloud computing system) using the communication module 1002 and send the alarm condition to the computing system through the end-to-end connection.

Based on the severity of the alarm condition and/or the alarm profile corresponding to the alarm condition, an alarm may be generated and/or annunciated that is associated with the severity of the alarm condition and/or the type of alarm condition. Different alarm conditions and/or alarm profiles may result in different types of alarms or different annunciations of the alarm. For example, an alarm associated with the highest severity level may include an audible alarm with a loudness that exceeds a particular decibel level (e.g., above 70 or 80 decibels), a visible alarm (e.g., a flashing or steady light) with a luminance above a particular luminance value (e.g., a luminance between 10⁵ or 10⁶ candelas per square meter), and/or a vibrational alarm. Further, the alarm associated with the highest severity level may not be snoozed or dismissed. Alternatively, the alarm associated with the highest severity level may be snoozed for a shorter time period than alarms of lower severity levels (e.g., for 5 minutes, for 10 minutes, etc.). An alarm associated with a different severity level than the highest severity level may include a different combination of audible, visible, and vibrational alarms. Not only may the existence of audible, visible, and vibrational alarms differ for different severity levels, but so may the characteristics of each of the alarm types. For example, audible alarms may have different sound patterns, loudness, frequencies, etc. Visible alarm may be of different intensity, color, pattern, etc. Vibrational alarms may be of different pattern, intensity, etc. Further, an alarm with a different severity level than the highest severity level may be permitted to be snoozed or dismisses, or snoozed for a longer period of time. In some examples, the severity of the alarm condition may determine the type of type of the alarm generated (e.g., audio, text, graphical, or any combination of these).

Further, the display of alarm conditions on the user interface may include an icon for each type of alarm condition. The user interface may display the number of alarm conditions and/or the number of alarm conditions of a particular type or severity level. In some cases, a duplicate alarm may be omitted from the list of alarms. In other cases, a count of the occurrence of alarms may be increased to reflect the duplicate alarm. In some cases, a duplicate alarm may result in the annunciation of the duplicate alarm. In other cases, the duplicate alarm is ignored. In some cases, the occurrence of a duplicate alarm may cause an escalation of the existing alarm. For example, if an alarm condition that causes an annunciation of an alarm with a first severity level is detected as occurring a second time, the alarm may be annunciated with a second severity level that indicates a greater degree of severity that the first severity level. It should be understood that an alarm occurring after an alarm condition is resolved may not be considered a duplicate alarm, but instead may be a reoccurrence of the alarm condition and/or an indicator that the resolution for the alarm condition failed (e.g., an insulin cartridge replacement is faulty or is empty).\

In some cases, the list of alarms may be accessed when the ambulatory medicament device is locked. Further, details about the alarms may be accessible when the ambulatory medicament device is locked.

Each of the alarm conditions, or information associated therewith, may be added to an indicator or user interface (e.g., a list, or other data structure or user interface element) that may be accessed by a user. This user interface may maintain the alarm condition on the user interface until the alarm condition is resolved. Further, the alarm conditions may be sorted or ranked based on the severity level of the alarm condition, the time that the alarm condition occurred, whether the alarm condition relates to the subject or the ambulatory medicament device, any combination of the foregoing, or any other factor for sorting or ranking the alarm conditions.

In some cases wherein the alarm is presented on a display, the displayed information may include details about what caused the alarm, the severity of the alarm, how to respond to or address the alarm, or any other information that may be informative regarding why the alarm was generated and/or how to respond to the alarm. In some cases, the information may provide a workflow or instructions on how to respond to the alarm. The instructions may include a link to a workflow provided by a manufacturer of the ambulatory medicament device or of another entity, such as an entity that provides medicament or site changing kits.

In some cases, different views of an alarm or different information associated with the alarm may be provided based on an identity of the user, or a role of the user, viewing the alarm. For example, a child may be instructed to contact a parent to address an alarm. But a parent may be provided with information to resolve the alarm. The parent may receive simplified information (e.g., blood glucose level is high) about what caused the alarm, but a healthcare provider may receive more detailed information regarding the alarm (e.g., internal control parameter values, insulin flow rates, curvature of insulin diminishment predictions, etc.) that facilitates the healthcare provider caring for the subject.

The alarm conditions may be displayed on a display of the ambulatory medicament device. Alternatively, or in addition, the alarm conditions may be displayed on a remote display that is separate from the ambulatory medicament device. The remote display may be a display that is authenticated or associated with a computing device that is authenticated to access data, such as alarm conditions, from the ambulatory medicament device. In some cases, the list of alarms may be presented on a mobile device (e.g., a smartwatch or smartphone) or on a computing device (e.g., a laptop or desktop) that can obtain data directly or indirectly from the ambulatory medicament device.

In some cases, annunciating the alarm may include contacting a manufacturer and/or user (e.g., a healthcare worker, a parent or guardian, or other registered user). Further, the alarm may include instructions on repairing the ambulatory medicament device and/or on addressing the alarm condition. For example, the alarm may provide a user with instructions to replace the insulin cartridge and how to replace the insulin cartridge. As another example, the alarm may provide instructions on how to change the battery of the device or on how to change a site where the insulin pump connects to the subject. In some cases, the alarm may include one or more operations associated with the alarm. For example, the alarm may trigger reordering of insulin or may request that the user confirm a reorder request to reorder insulin.

A user may be able to acknowledge and/or snooze alarms. Certain alarms, such as informational alarms, may be dismissible. However, generally the alarm may remain on the alarm list until the condition that caused the alarm is resolved.

Resolving the alarm may include any action that addresses the condition that caused the alarm to be generated. For example, resolving the alarm may include replacing an insulin cartridge, changing a site where the ambulatory medicament device is connected to the subject, charging a battery of the ambulatory medicament device, providing insulin or a counter-regulatory agent to the subject and/or the ambulatory medicament device, or any other action that may be performed to address an alarm condition. In some cases, the resolution action may be acknowledging the alarm. For example, if the alarm is informational (e.g., to inform the user that more insulin has been ordered), acknowledging the alarm may be a sufficient resolution action.

In some cases, whether the alarm condition is resolved may depend on an identity of the user. For example, if a child interacts with an alarm related to reordering of insulin, the alarm may remain until a parent or guardian acknowledges the alarm. However, the child may be able to snooze the alarm. In some cases, a user interface that displays alarms may differ based on who is viewing the alarm. For example, a child may view the alarms, but may not be able to interact with the alarms. However, a parent or guardian may be able to snooze or dismiss an alarm. Further, a child may be instructed to bring the device to a parent or adult to address an alarm. In some cases, the child may be informed of how urgently to contact the parent (e.g., contact a parent immediately, within a day, within a week, etc.). Moreover, a designated adult may separately be alarmed (e.g., via a text or email alarm). The parent or guardian may receive additional information not provided to the child or subject (e.g., a link to repair instructions or a workflow to address the alarm condition).

In some cases, certain conditions may self-resolve over time. For example, a low battery alarm may resolve as the battery is charged. In such cases, the alarm may be cancelled automatically as the battery charge level exceeds a particular threshold. Further, in some cases, one or more alarms and/or the alarm list can be viewed and/or accessed on a home screen, a main screen, or other non-alarm based user interface screen in addition to a user-interface screen designated for display alarms. The alarm list may be accessed from the ambulatory medicament device and/or a computing system in communication with the ambulatory medicament device.

However, in some cases, the alarm condition may or may not be resolvable when the ambulatory medicament device is locked.

A user may interact with the alarms generated based on the alarm condition. In some cases the user can only interact with the alarms when the ambulatory medicament device is unlocked. In other cases, the user can interact with the alarms to snooze them or to obtain further information. However, the user may not be able to dismiss the alarm without unlocking the ambulatory medicament device. Interacting with the alarms may include providing information associated with the alarm to a user in response to the user interacting with the alarm, or an indicator representative of the alarm.

### Example Implementation of Glucose Level Control System

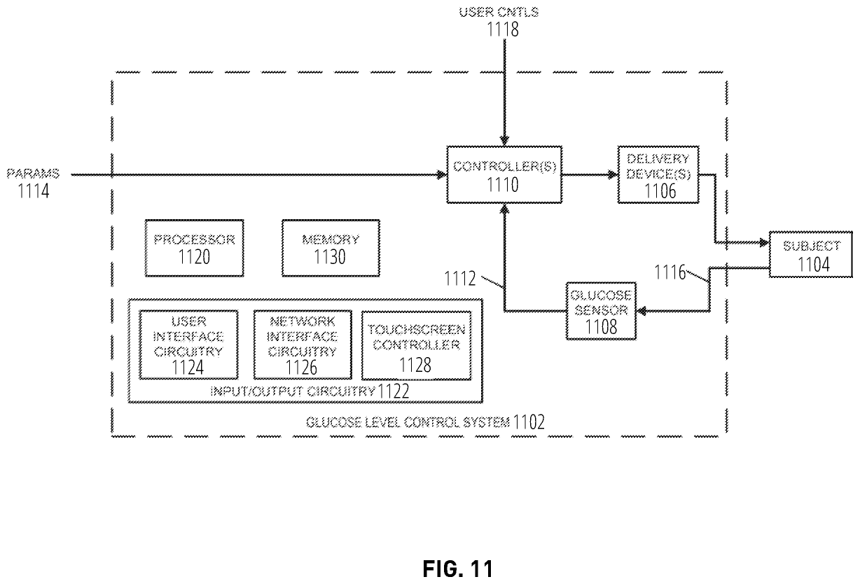

FIG. 11 illustrates a glucose level control system 1102 for regulating the blood glucose level of an animal subject (subject 1104), which may be a human. The glucose level control system 1102 is an example of a medicament infusion system and may include any of the embodiments previously described above with respect to medicament infusion systems.

The subject 1104 may receive doses of insulin from one or more delivery device(s) 1106, for example infusion pump(s) coupled by catheter(s) to a subcutaneous space of the subject 1104. As described below, the delivery device(s) 1106 may also deliver a counter-regulatory agent or hyperglycemic agent, such as glucagon or dextrose, for control of the blood glucose level under certain circumstances. For the delivery of both insulin and a counter-regulatory agent (e.g., glucagon), the delivery device(s) 1106 may be mechanically driven infusion mechanisms having dual cartridges for insulin and the counter-regulatory agent, respectively. In the present description, reference is made to glucagon specifically, but it is to be understood that this is for convenience only and that other counter-regulatory agents (e.g., dextrose) may be used. Similarly, the term “insulin” herein is to be understood as encompassing all forms of insulin-like substances including natural human or animal insulin as well as synthetic insulin in any of a variety of forms (commonly referred to as “insulin analogs”).

For online or autonomous operation, a glucose sensor 1108 is operatively coupled to the subject 1104 to continually sample a glucose level of the subject 1104. In some cases, the glucose sensor 1108 may be referred to as a continuous glucose monitoring (CGM) sensor, which may continuously or periodically measure or sense blood glucose levels of the subject 1104 for at least a period of time. Sensing may be accomplished in a variety of ways, generally involving some form of physical coupling 1116 between the subject 1104 and the glucose sensor 1108. A controller 1110 may control operation of the delivery device(s) 1106 as a function of a glucose level signal 1112 from the glucose sensor 1108 and subject to programmed input parameters (PARAMS) 1114 which may be provided by a user such as the subject 1104, a parent or guardian of the subject 1104, or a healthcare provider (e.g., a clinician or doctor). One input parameter for automatic operation may include the weight of the subject 1104. In some cases, the glucose level control system 1102 can provide effective automated control without receiving explicit information regarding either meals that the subject 1104 has ingested or any other “feedforward” information, which is achieved in part by an adaptive aspect to operation of the controller 1110. In other cases, the glucose level control system 1102 can use received information regarding either meals that the subject ingested, or plans to ingest, or other “feedforward” information to modify control of blood glucose and/or delivery of insulin or counter-regulatory agent.

The controller 1110 is an electrical device with control circuitry that provides operating functionality as described herein. In one embodiment, the controller 1110 may be realized as a computerized device (e.g., a hardware processor) having computer instruction processing circuitry that executes one or more computer programs each including respective sets of computer instructions. In some cases, the processing circuitry will generally include one or more separate processors 1120 along with memory 1130 and input/output circuitry 1122 coupled to or in communication with the separate processor 1120 (or separate processors 1120), where the memory 1130 stores computer program instructions and data, and the input/output circuitry 1122 can provide interface(s) to external devices such as the glucose sensor 1108 and delivery device(s) 1106. In some cases, the input/output circuitry 1122 may provide a user interface, or may operate with one or more processors (e.g., the controller 1110 or a separate processor 1120 included in the glucose level control system 1102 or in a separate computing system, such as a smartphone, a laptop computer, a desktop computer, a smartwatch, and the like) to provide a user interface to a user (e.g., the subject 1104, a parent or guardian, or a clinician). In some cases, the input/output circuitry 1122 may include a touchscreen and/or a touchscreen controller 1128 configured to control a touchscreen (not shown).

In some cases, the controller 1110 may perform all of the functionality of the glucose level control system 1102. In such cases, the processor 1120 may be optional or omitted. In other cases, the controller 1110 may perform at least automated glucose control of the subject 1104, and one or more separate processors 1120 may perform one or more additional operations of the glucose level control system 1102 (or medicament pump), such as tracking occurrences of hyperglycemic or hypoglycemic events or risk events, outputting data to a user, controlling or initiating communication with another computing system, regulating access to the glucose level control system 1102, or other operations unrelated to operation of a medicament pump or the delivery device(s) 1106.

The input/output circuitry 1122 may control communication with one or more other computing systems and/or with a user. In some cases, the input/output circuitry 1122 may include one or more separate interface circuits or controllers to facilitate user interaction and/or communication. For example, the input/output circuitry 1122 may include user interface 1124, network interface 1126, and/or a touchscreen controller 1128.

The user interface 1124 may include any circuitry or processors that may output a user interface to a user and/or receive user input from the user via the user interface. The user interface 1124 may receive one or more signals from a separate processor 1120 corresponding to a user interface. The user interface 1124 may control a display to present the user interface to a user based on the one or more signals received from the separate processor 1120. Further, the user interface 1124 may include any circuitry that can receive a signal corresponding to an interaction by a user with a user interface and can provide the signal to the separate processor 1120 and/or controller 1110 for further processing. In some cases, the user interface circuitry may be replaced by a touchscreen controller 1128 that can control a touchscreen interface. In other cases, the touchscreen controller 1128 may be in addition to the user interface 1124.

The network interface 1126 may include any circuitry that enables communication with a wired or wireless network. The network interface 1126 may include one or more network interface cards and/or wireless radios (e.g., a Bluetooth radio, a Bluetooth Low Energy (BLE) radio, a 4g LTE radio, a 5G radio, a ND-LTE radio, and the like).

The memory 1130 can include non-volatile memory and/or volatile memory. The non-volatile memory may include flash memory or solid-state memory.

The glucose level control system 1102 is also able to operate in an offline manner in which it is used to provide delivery of insulin (and potentially glucagon as well), independent of or without receipt of glucose levels reported by the glucose sensor 1108. For example, in cases where the glucose sensor 1108 needs replacing, is not properly connected to the subject 1104, or is defective, the glucose level control system 1102 may operate in an offline manner without input from the glucose sensor 1108. Thus, overall operation may be divided between online periods each including a succession of sampling intervals when a glucose level signal 1112 is available, and offline periods each including a succession of sampling intervals when the glucose level signal 1112 is either completely or intermittently unavailable. The description below uses the terms “online” and “offline” for these periods. Also, offline operation may be user-selected for some reason even when a glucose level signal 1112 is available for use.

User control inputs (user control inputs 1118 or USER CNTLs) may be provided via a local or remote user interface of some type. In some embodiments, the user interface may resemble that of conventional insulin pumps or similar devices, e.g., by including control buttons for commanding the delivery of a bolus and perhaps a small display. In other embodiments, the system may have a wired or wireless interface to a remote device that may incorporate a fuller-function user interface, such as a smartphone, smartwatch, laptop computer, desktop computer, cloud computing service, or other wearable device or computing device. In some cases, the wireless interface may provide access to a local area network, such as a personal home network, a company network, or otherwise. Alternatively, or in addition, the wireless interface may provide a direct connection between local devices available to a user (e.g., via Bluetooth or other near field communication technologies). In some cases, the wireless interface may provide access to a wide area network, such as, but not limited to, the Internet. For example, the wireless interface may include a cellular interface that permits access to a network via a 4G or 5G cellular connection. In some cases, the cellular interface may be a low power interface, such as narrowband LTE or other Internet of Things (IoT) interfaces.

In offline mode, the glucose sensor 1108 may be absent, non-functioning, or not coupled to the subject 1104. As such, in offline mode, the blood glucose glucose level signal 1112 may not be available to control automatic operation. In some cases, a user may provide one or more blood glucose measurements to the glucose level control system 1102 to facilitate automatic operation of the control system 1102. These measurements may be provided over a particular time period. Alternatively, or in addition, the glucose level control system 1102 may use a therapy history and/or a history of prior glucose control measurements to facilitate automatic operation of the glucose level control system 1102 for at least a particular time period.

The description herein refers to a “user” as the source of the user control inputs 1118. The “user” as used herein may be the subject 1104, a parent or guardian of the subject 1104, a healthcare provider (e.g., a clinician, doctor, or other person who may provide medical care to the subject), or any other user who may be authorized to help manage therapy of the subject 1104. In certain implementations, the glucose level control system 1102 is a personal device worn by a subject 1104 for continual glucose control. In some such implementations, the user and subject 1104 may be the same person. In other implementations, there may be another person involved in the care of the subject 1104 and providing control input, and in such implementations, that other person has the role of user.

### Example Controllers for a Glucose Level Control System

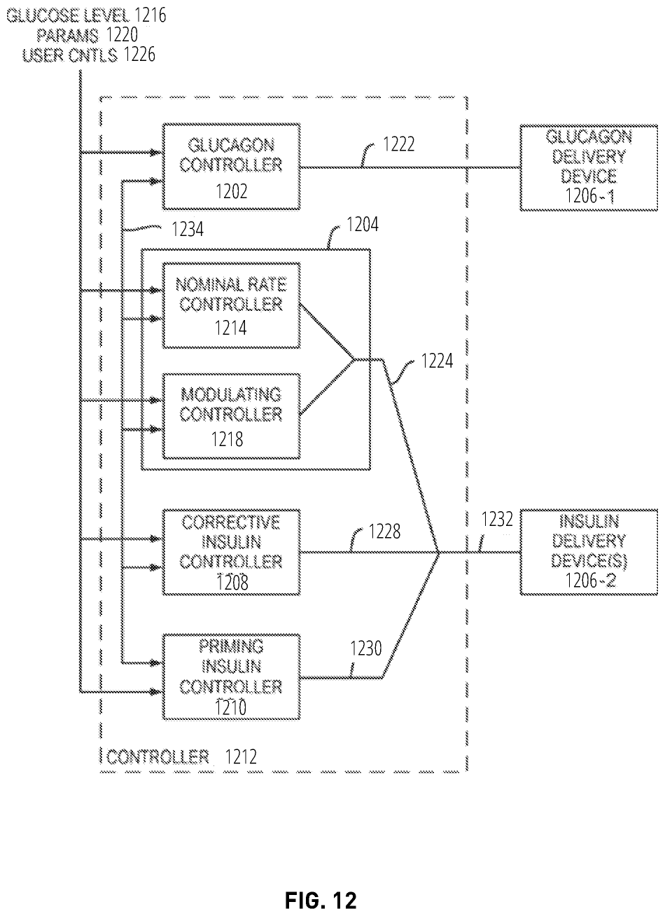

FIG. 12 shows an example structure of the controller 1212 in accordance with certain embodiments. The controller 1212 illustrated in FIG. 12 may represent a physical structure with different controllers or processors, or a logical structure that is implemented by one or more physical processors. In other words, a single processor may be used to implement each of the controllers illustrated in FIG. 12, each controller may be implemented by its own processor, or certain processors may implement multiple, but not necessarily all, of the controllers illustrated in FIG. 12 as part of the controller 1212. Moreover, although the controllers of FIG. 12 are illustrated as part of the controller 1212, in some implementations, one or more of the controllers may be separate from the controller 1212.

The controller 1212 may include four separate controllers, namely a glucagon (or counter-regulatory agent) glucagon controller 1202, a basal insulin controller 1204, a corrective insulin controller 1208 (or model predictive controller), and a priming insulin controller 1210 (or meal controller). The basal insulin controller 1204 includes a nominal rate controller 1214 and a modulating controller 1218. As shown, the glucagon controller 1202 generates a glucagon dose control signal 1222 provided to a glucagon delivery device 1206-1. Respective outputs 1224, 1228, and 1230 from the controllers 1204, 1208, and 1210 may be combined to form an overall insulin dose control signal 1232 provided to insulin delivery device(s) 1206-2. As shown, the output signal 1224 from the basal insulin controller 1204 may be formed by a combination of respective outputs of the nominal rate controller 1214 and a modulating controller 1218. The insulin delivery device(s) 1206-2 may include devices tailored to deliver different types and/or quantities of insulin, and the exact configuration may be known to and/or under the control of the controllers 1204-1210. For ease of description, the collection of one or more insulin delivery device(s) 1206-2 is referred below to in the singular as an insulin delivery device 1206-2.

Also shown in FIG. 12 are input/output signals of the various controllers, including the glucose level signal 1216, programmed input parameters (PARAMS)s 1220 and user user control inputs 1226 as well as a set of inter-controller signals 1234. The inter-controller signals 1234 enable communication of information from one controller, where the information is developed or generated, to another controller where the information may be used for that controller's control function.

The controllers 1202-1210 may be operated in either the online/automatic mode or in the offline mode. In the automated mode, the corrective insulin controller 1208 regulates glucose level using a control scheme such as described in U.S. Pat. No. 7,806,854, the contents of which are hereby incorporated by reference in its entirety herein. The basal basal insulin controller 1204 and priming insulin controller 1210 may perform adaptive automated control as described in International Patent Application Publication WO 2012/058694 A2, the contents of which are hereby incorporated by reference in its entirety herein. The controllers 1202-1210 generally employ control methods or algorithms that include control parameters that are mathematically combined with reported glucose values to generate an output value that is converted (either directly or via additional conditioning) into the dose control signals 1222, 1232. For example, the control scheme described in U.S. Pat. No. 7,806,854 includes a generalized predictive control (GPC) method that incorporates a variety of control parameters. The control algorithms are generally adaptive, meaning that control parameters are dynamically adjusted during operation to reflect changing operating circumstances and a “learning” aspect—by monitoring its own operation, the algorithm adjusts its operation to be more specifically tailored to the individual user, enhancing the algorithm's effectiveness and reducing or avoiding a need for additional explicit input information about the user. It should be noted that the programmed input parameters (PARAMS)s 1220 may form part of the control parameters used by the control algorithm. Other control parameters are internal parameters according to the specifics of the algorithm, and selected ones of those internal control parameters are dynamically adjusted to realize the adaptation of the control algorithm.

One feature of operation is the ability of the controllers to learn from recent past periods of online operation and to use that learning during offline operation. U.S. Pat. No. 10,543,313, the contents of which are hereby incorporated by reference in its entirety herein, describes two methods that are usable independently or together in offline operation. A first method automatically calculates the correct size of a correction bolus of insulin at a time of receiving an isolated glucose measurement, the correction bolus then being administered by the system in response to a user control input. A second method automatically calculates the correct size of a meal bolus of insulin and administers it in response to a user control input. Both methods utilize information obtained during past periods of online operation to automatically calculate correct values, freeing the user of a need to make the calculation or provide a correction factor.

### Carbohydrate Therapy Equivalence Tracking

Hyperglycemia is a condition that occurs when the levels of sugar or glucose in the blood exceeds a particular level (e.g., 180 mg/dL). This condition may occur in diabetics. To help reduce the occurrence of hyperglycemia, a subject may use an automated glucose level control system, which may automatically provide insulin to a subject using a medicament pump. The administered insulin may help control the blood glucose level of the subject by consuming glucose in the subject.

Hypoglycemia is a condition that occurs when the levels of sugar or glucose in the blood are below a particular level (e.g., 70 mg/dL). This condition may have adverse consequences including loss of consciousness, seizures, and death. The levels of blood sugar that lead to hyperglycemia and hypoglycemia may vary from patient to patient. To reduce the risk of hypoglycemia, a subject may consume carbohydrates to increase blood sugar. Because of the severe consequences associated with a hypoglycemic event, subjects usually consume carbohydrates that metabolize quickly. These carbohydrates are often unhealthy but are preferable to the occurrence of a hypoglycemic event. For example, the carbohydrates may include candy bars with a lot of refined sugar.

A bihormonal glucose-control system may reduce the risk of occurrence of hypoglycemia by including, in addition to insulin, a counter-regulatory agent (e.g., Glucagon) that can be administered to a subject when the blood glucose level drops too low (e.g., below 50 mg/dL). For subjects who do not have a bihormonal glucose-control system, it may be useful to understand the reduction in carbohydrate therapy, or the consumption of carbohydrates to address hypoglycemic events or potential hypoglycemic events, that can be achieved by switching to a bihormonal glucose-control system. Further, it may be useful for subjects who do have a bihormonal glucose-control system to understand the reduction in carbohydrate therapy obtained by having the bihormonal glucose-control system. For example, understanding the amount of carbohydrate therapy consumed or avoided can be important in monitoring the subject's nutrition intake. While monitoring nutrition in take is important for all people, it is particularly important for diabetics because diabetics must balance eating healthy with ensuring that their blood sugar is maintained in a particular range to avoid both hyperglycemia and hypoglycemia.

The present disclosure relates to a system that can perform a computer-implemented method of generating an indication of total carbohydrate therapy over a time period in a subject using a medicament pump configured to deliver at least insulin therapy to the subject. The system may be an automated glucose level control system (e.g., the glucose level control system 1102) that includes a hardware processor (e.g., controller 1212) for determining dose control signals to provide the medicament pump (e.g., delivery device(s) 1206). In some cases, the medicament pump may be configured to deliver both insulin therapy and counter-regulatory agent (e.g., Glucagon) therapy. Alternatively, the system may be separate from the glucose level control system but may receive blood glucose information from the glucose level control system. For example, the system may be personal computing system or a cloud computing system that can received blood glucose information from the glucose level control system.

The system may receive or determine a glucose level of a subject (e.g., subject 1104). The glucose level of the subject may be determined based on a signal (e.g., a glucose level signal) received from a continuous glucose monitoring (CGM) sensor (e.g., glucose sensor 1108) that corresponds to the glucose level of the subject. In some cases, the glucose level may be determined from an isolated glucose measurement, such as may be obtained using a glucose measurement kit and/or glucose paper.

Using at least the glucose level of the subject, the system can determine whether a triggering event for raising the subject's blood glucose level has occurred. The triggering event may include a blood glucose level that indicates an occurrence of a hypoglycemic event or a risk of the occurrence of a hypoglycemic event exceeding a risk threshold within a particular period of time. A risk of a hypoglycemic event may be determined when a glucose level of the subject falls below a glucose threshold. This glucose threshold may vary for different subjects and may, in some cases, be specified by the subject or a caregiver (e.g., healthcare provider, parent, or guardian). Thus, in some cases, different triggering events may be defined based on a risk tolerance of a subject to an occurrence of hypoglycemia or to possible different preferences for an amount of blood glucose to be present in the subject. Different subjects may prefer that blood glucose be maintained, or attempt to be maintained, at different levels due, for example, to differences in activity levels or metabolism by different subjects. Determining the risk of the occurrence of a hypoglycemic event may include receiving an indication of a risk of hypoglycemia from a glucose sensor or a prediction of a glucose level at a future time. For example, a determination of an imminent risk of hypoglycemia may comprise a determination that the subject's blood glucose level is expected to be below 60 mg/dl within the next 5-15 minutes.

Responsive to the triggering event, the system may determine an amount of counter-regulatory agent to administer, or an amount of counter-regulatory agent that would be administered if the glucose level control system included the capability of administering a counter-regulatory agent. In some cases, the counter-regulatory agent is administered by, for example, the automated glucose level control system. In other cases, the counter-regulatory agent is not administered. For example, the automated glucose level control system may not be capable of delivering the counter-regulatory agent. As another example, the automated glucose level control system may be capable of delivering the counter-regulatory agent but may not have a dose of the counter-regulatory agent available.

The system can use the indication of the counter-regulatory agent that is administered or that would be administered to determine a corresponding amount of carbohydrates. The corresponding amount of carbohydrates may be indicative of the amount of carbohydrates that were consumed to prevent the hypoglycemic event, to reduce the risk of the hypoglycemic event, or in response to an occurrence of a hypoglycemic event. Alternatively, or in addition, the corresponding amount of carbohydrates may be indicative of the amount of carbohydrates that would have been consumed if the counter-regulatory agent were not available.

The corresponding amount of carbohydrates may be obtained from a mapping between amounts of a counter-regulatory agent and amounts of carbohydrates. In some cases, the mapping may be based on a measured equivalency between carbohydrates and a counter-regulatory agent. Alternatively, or in addition, the mapping may be between a determined amount of counter-regulatory agent and an amount of carbohydrate a subject indicates he or she normally consumes when determining that a hypoglycemic event may occur.

The mapping may be implemented by a lookup table that maps different amounts of counter-regulatory agent to different corresponding amounts of carbohydrates. In some cases, a single quantity of counter-regulatory agent may map to different amounts of carbohydrates depending on the type of carbohydrate consumed (e.g., simple vs complex carbohydrates, or the type of candy bar consumed, etc.). Alternatively, the mapping may be based on a formula that converts an amount of counter-regulatory agent to an amount of carbohydrates based on a correspondence between the amount of counter-regulatory agent and the amount of carbohydrates. The determination of a relationship between the counter-regulatory agent and carbohydrates may be based on clinical tests comparing carbohydrates to the counter-regulatory agent (e.g., Glucagon, dextrose, etc.). Further, the mapping may be based at least in part on a subject's preferred carbohydrate source and/or characteristics of the subject (e.g., weight).

In some cases, the system can track a number of hypoglycemic events or a number of occurrences of a trigger indicating an impending risk of a hypoglycemic event within a particular time period. The time period may be days, weeks, months, years, or any other period of time over which it is desirable to determine a relationship between carbohydrates consumed or avoided based on the lack of availability or availability of a counter-regulatory agent. In some cases, the tracking of carbohydrate therapy may be based on a number of hypoglycemia events or hypoglycemia risk events instead of or in addition to a time period.

For each occurrence of a hypoglycemic event or occurrence of a trigger indicating an impending risk of a hypoglycemic event, the system can determine an estimate of the carbohydrate therapy saved or that would have been saved by having access to the counter-therapy agent. The system can generate a report for the time period that indicates the total carbohydrate saved or that would have been saved with access to counter-regulatory agent. The report may include an aggregate or sum of the carbohydrate therapy required or saved during the time period. This time period may be days, weeks, months, years, or since a particular time (e.g., since the subject starting using the system). Further, the report may indicate the type of carbohydrates typically consumed by the subject when responding to a hypoglycemic event or a risk of an impending hypoglycemic event. This report can be presented to the subject, a healthcare provider, and/or a parent or guardian of the subject. The healthcare provider can use this report to help care for the subject. For example, the healthcare provider can use the report to generate a nutrition plan for the subject that accounts for the carbohydrates consumed to maintain the blood glucose level within a desired or setpoint range.

The report may include a range of carbohydrate therapy avoided or likely consumed to address the risk of hypoglycemia events. Further, the report may include an amount of calories saved or not consumed, an amount of sugar avoided, an amount of food not consumed, a likely weight gain avoided, etc. based on the use of a counter-regulatory agent in place of carbohydrate therapy.

### Carbohydrate Therapy Equivalence Tracking Process

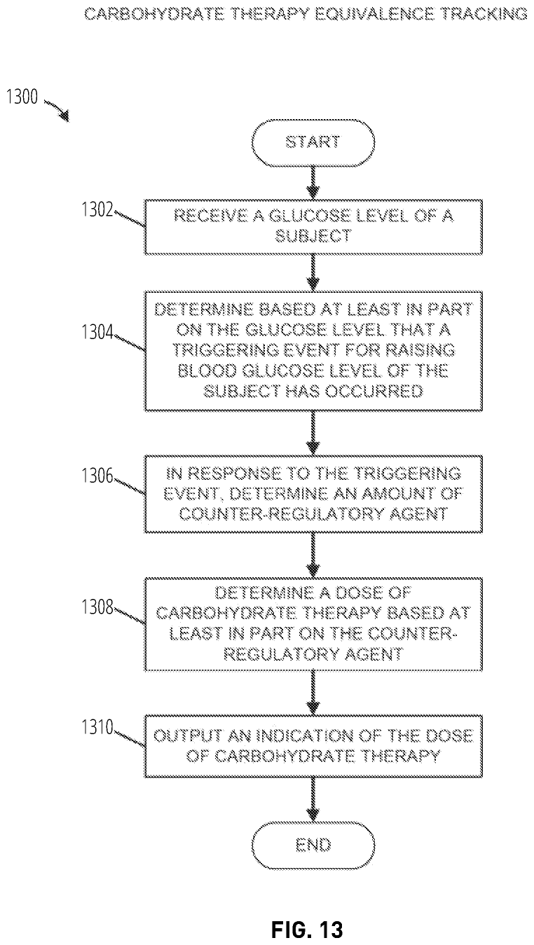

FIG. 13 presents a flowchart of a carbohydrate therapy equivalence tracking process 1300 in accordance with certain embodiments. The process 1300 may be performed by any system that can track the glucose level of a subject over time and identify hypoglycemic events, or occurrences when a risk of a hypoglycemic event satisfies a threshold (e.g., when the risk of the hypoglycemic event matches or is above a particular probability). For example, the process 1300 may be performed by one or more elements of the glucose level control system 1102. In some cases, at least certain operations of the process 1300 may be performed by a separate computing system that receives indications of blood glucose levels of the subject 1104 from the glucose level control system 1102 and/or indications of hypoglycemic events (or identified above threshold hypoglycemic risk events). Although one or more different systems may perform one or more operations of the process 1300, to simplify discussions and not to limit the present disclosure, the process 1300 is described with respect to particular systems.

The process 1300 begins at block 1302 where the glucose level control system 1102 receives a glucose level of a subject 1104. Receiving the glucose level may include receiving a glucose level signal corresponding to a glucose level of the subject. The glucose level signal may be received from the glucose sensor 1108 (e.g., a CGM sensor). Alternatively, or in addition, the glucose level may be received from a user that provides the glucose level to the glucose level control system 1102 via a user interface, such as a user interface generated by the separate processor 1120 that may be output on a touchscreen by the touchscreen controller 1128. The glucose level received from the user may be a glucose level measured using an alternative sensor or measurement mechanism (e.g., diabetes measurement strips) that may be used in place of the glucose sensor 1108.

At bock 1304, the glucose level control system 1102 determines based at least in part on the glucose level that a triggering event for raising the blood glucose level of the subject 1104 has occurred. The triggering event may include a determination that a hypoglycemic event or an episode of hypoglycemia is present or is occurring in the subject 1104. Alternatively, or in addition, the triggering event may include a determination that there is an impending risk of hypoglycemia in the subject 1104, or an impending risk that a hypoglycemic event will occur within a particular amount of time in the subject 1104. The determination of the hypoglycemic event or the risk of a hypoglycemic event occurring may be determined by comparing the glucose level of the subject to a glucose threshold. Alternatively, or in addition, the determination of the hypoglycemic event or the risk of a hypoglycemic event occurring may be determined by comparing a trend and/or rate of change (e.g., rate of decrease) in the glucose level to a threshold. In some cases, the particular blood glucose level and the trend in the blood glucose level may be combined to determine a risk of hypoglycemia. For example, if the glucose level is low (e.g., below a particular threshold, such as 60 mg/dL), but a determined trend in the glucose level is upwards, then a risk of hypoglycemia may be lower than if the glucose level is above the threshold, but the determined trend in the glucose level is downwards towards a threshold. In some cases, the threshold(s) used to determine whether a hypoglycemic event is occurring or to determine that there is an above threshold risk of hypoglycemia occurring may vary based on physiological characteristics of the subject 1104. The physiological characteristics may be based on physiological characteristics associated or shared among groups of patients (e.g., gender, age, weight) or may be specific to the particular subject 1104. For example, thresholds associated with a risk of hypoglycemia may be determined based on determined glucose levels of the subject 1104 during prior occurrences of hypoglycemia as determined by the glucose level control system 1102 or based on clinical data specific to the subject 1104.

In response to the triggering event at the block 1304, the glucose level control system 1102 determines an amount of counter-regulatory agent at block 1306. The glucose level control system 1102 may determine the amount of counter-regulatory agent based at least in part on the blood glucose level of the subject 1104, the amount or percentage of risk of hypoglycemia occurring (e.g., a 99% risk or probability of hypoglycemia may trigger a larger counter-regulatory agent dose than a 75% risk or probability of hypoglycemia), physiological characteristics of the subject 1104, a trend in the blood glucose level of the subject 1104, or a type of counter-regulatory agent.

In some cases, the glucose level control system 1102 may use a delivery device(s) 1106-1 to deliver the determined amount of counter-regulatory agent to the subject 1104. The counter-regulatory agent may be delivered to the subject 1104 in response to the impending risk of hypoglycemia or the episode of hypoglycemia, and/or in response to the glucose level satisfying or falling below a threshold glucose level. The threshold glucose level or the determination of whether to deliver the counter-regulatory agent may be based on physiological characteristics of the subject 1104 and/or the risk tolerance of the subject 1104 to a hypoglycemic event. It should be understood that, in the present context, risk tolerance generally does not refer to a user's subjective propensity for risk. Instead, the risk tolerance is typically an objective determination of how likely the subject 1104 is to have a hypoglycemic event, or for symptoms of hypoglycemia to occur, when the blood glucose level of the subject 1104 is at a particular level. This risk tolerance may be determined based on a history of hypoglycemia, or lack thereof, in the subject 1104 at particular blood glucose levels and/or based on clinical data obtained for the subject 1104.

In other cases, the glucose level control system 1102 may not deliver counter-regulatory agent to the subject 1104 because, for example, the glucose level control system 1102 may not be capable of delivering counter-regulatory agent or because the cartridge holding the counter-regulatory agent may be empty or have less than a threshold amount of counter-regulatory agent remaining.

At block 1308, the glucose level control system 1102 determines a dose of carbohydrate therapy based at least in part on the counter-regulatory agent. The carbohydrate therapy may refer to carbohydrates consumed to prevent or respond to an occurrence of hypoglycemia. The carbohydrates may include any type of carbohydrate that the subject 1104 may consume to prevent or respond to an occurrence of hypoglycemia, and typically includes fast-acting carbohydrates, which may include sugary foods that are easily converted into sugars in the human body. For example, the carbohydrate may be a candy bar, soda, fruit juice, or other foods that may have a lot of sugar or refined sugars.

Determining the dose of carbohydrate therapy may include accessing a mapping between the counter-regulatory agent and carbohydrates. This mapping may be stored in, and accessed from, the memory 1130 and/or may be accessed from another computing device. The glucose level control system 1102 may determine the dose of carbohydrate therapy based at least in part on the mapping and the amount of the counter-regulatory agent. In some cases, the mapping may vary based on the type of counter-regulatory agent and/or the type of carbohydrates. The type of counter-regulatory agent may be identified by a user or may automatically be determined based on a medicament cartridge installed or inserted into the glucose level control system 1102. Further, the type of carbohydrates may be specified by a user and may include an identity of the type of carbohydrates usually consumed by the subject 1104 when responding to an occurrence or a risk of an occurrence of hypoglycemia. For example, the user may specify, via a user interface, whether the subject usually consumes a candy bar or fruit juice, or the size of the carbohydrate usually consumed when responding to an occurrence or a risk of an occurrence of hypoglycemia.

In some cases, the mapping between the counter-regulatory agent and carbohydrates may be generated based on a clinical comparison of the counter-regulatory agent to the carbohydrates. Alternatively, or in addition, the mapping may be based at least in part on a physiological characteristic of the subject 1104.

The mapping may be stored in a lookup table or other data structure that can store relationships between different carbohydrates and counter-regulatory agents. The mapping may be between different quantities and/or types of carbohydrates and different quantities and/or types of counter-regulatory agent. Alternatively, or in addition, the mapping may be a formula that relates the carbohydrates to the counter-regulatory agent or vice versa. For example, the glucose level control system 1102 may use the determined amount of counter-regulatory agent as an index to a lookup table to determine a corresponding quantity of carbohydrates. Alternatively, the glucose level control system 1102 may apply the determined amount of counter-regulatory agent to a formula to calculate a corresponding quantity of carbohydrates. This formula may be generated based on the type of counter-regulatory agent and/or carbohydrates, physiological characteristics of the user, and/or clinical data.

In some cases, the mapping may vary based on the glucose level control system 1102. For example, the glucose level control system 1102 may include a first mapping when the glucose level control system 1102 (or medicament pump thereof) is a bi-hormonal pump configured to deliver insulin and counter-regulatory agent therapy to the subject, and may include a second mapping when the glucose level control system 1102 is not configured to deliver the counter-regulatory agent therapy to the subject 1104. In some cases, the glucose level control system 1102 may store both mappings in the memory 1130. For example, the glucose level control system 1102 may use the first mapping when counter-regulatory agent is available and may use the second mapping when counter-regulatory agent is not available. The mappings may vary for a number of reasons including because a bi-hormonal glucose level control system 1102 may more precisely control the occurrence of hypoglycemic events due to the availability of counter-regulatory agent, which may therefore alter the frequency and type of carbohydrates that a subject may consume.

At block 1310, the glucose level control system 1102 outputs an indication of the dose of carbohydrate therapy. Outputting the indication of the dose of carbohydrate therapy may include outputting an indication of the dose of carbohydrate therapy on a display for presentation to a user. Further, the indication of the dose of carbohydrate therapy may be transmitted to another computing system for display or aggregation with other therapy data associated with the subject 1104, such as therapy data used by a clinician to help manager the subject 1104 care. In some cases, the indication of the dose of carbohydrate therapy may be included in a report corresponding to care of the subject 1104.

In certain embodiments, the operations of the process 1300 are performed or repeated over a period of time. For example, the operations associated with the block 1302-1308 may be repeated one or more times over the period of time. In such cases, the determined doses of carbohydrate therapy may be aggregated for the period of time to determine a total carbohydrate therapy for the period of time. Further, the block 1310 may include outputting an indication of the dose of carbohydrate therapy for each individual time that a dose of carbohydrate therapy is determined and/or the aggregated determined doses of carbohydrate therapy for the period of time. The period of time may be any time period. For example, the period of time may be a day, week, month, year, since the subject 1104 began using the glucose level control system 1102, since a user obtained or ceased obtaining access to a counter-regulatory agent, or any other period of time. In some cases, the period of time is defined by the occurrences of hypoglycemic events or occurrences of the risk of hypoglycemia satisfying a threshold. For example, the period of time may be the time associated with 5, 10, 15, 100, or any other number of hypoglycemic events or occurrences of the risk of hypoglycemia satisfying a threshold.

The indication of total carbohydrate therapy may correspond to a reduction in carbohydrates consumed by the subject 1104 because, for example, of the availability of counter-regulatory agent to the glucose level control system 1102, and consequently, the subject 1104. Thus, the indication of total carbohydrate therapy may correspond to a reduction in carbohydrates achievable by an availability to the subject 1104 of the counter-regulatory agent. Further, the indication of total carbohydrate therapy may correspond to an amount of counter-regulatory agent provided or that can be provided to the subject as a substitute for carbohydrates.

The particular carbohydrates consumed, or the amount of carbohydrates consumed by each subject or during each hypoglycemic event, may vary. For example, a subject 1104 may consume a particular candy bar when the measured blood sugar level of the subject 1104 is too low or when the subject feels that the blood sugar level is likely low (e.g., begins to feel some hypoglycemic effects). The subject may consume the whole candy bar or may consume a portion. Some of the candy bar may be lost to the subject (e.g., fall on the ground). In other cases, the subject may have different candy bars available, or other refined sugar sources, during different hypoglycemic events. Thus, even though there may be an objective mapping between carbohydrates and counter-regulatory agent, the amount of carbohydrates consumed or avoided due to the availability of counter-regulatory agent may vary for each hypoglycemic event. Accordingly, the indication of total carbohydrate therapy avoided, or that could be avoided if counter-regulatory agent were available, may indicate a range of carbohydrates that may potentially be replaced by the availability of counter-regulatory agent.

In some cases, the indication of carbohydrate therapy or total carbohydrate therapy may include one or more of an indication of calories, an indication of carbohydrates, an indication of a measure of sugar, an indication of a quantity of food, or an indication of weight of the subject attributable to the carbohydrate therapy. The indications may be associated with what is consumed due to a lack of counter-regulatory agent, or what is avoided based on the availability of counter-regulatory agent. For example, the indication of calories may be the number of calories not consumed because of the presence of the counter-regulatory agent. Advantageously, the availability of therapy information relating to the carbohydrate therapy or avoided carbohydrate therapy can assist in patient care. For example, a subject can reduce refined sugar consumption that can have a number of health consequences. Further, a healthcare provider can better help a subject control his or her weight based on the carbohydrate information.

The indication of carbohydrate therapy may be presented to a user in any presentable form. For example, the indication of carbohydrate therapy may be presented as a table, a chart, a graph, a histogram, or other data presentation tool for indicating the reduction in carbohydrates over time that is achieved by the presence of counter-regulatory agent or that could be achieved by the use of counter-regulatory agent for the particular subject 1104. It should be understood that the indication of carbohydrate therapy data may vary for different users due to differences in physiological characteristics of the users, differences in the diabetes of each user, differences in lifestyle of each user, among other factors. Advantageously, by using the glucose level control system 1102 to track the carbohydrate therapy of the subject 1104 or to determine the carbohydrate therapy avoided or avoidable associated with counter-regulatory agent, management of the blood glucose level of the subject 1104 can be personalized.

### Additional Carbohydrate Therapy Equivalence Tracking Embodiments

People with diabetes often consume oral carbohydrates for the purpose of treating or preventing hypoglycemia. Such extra carbohydrates can have unhealthy consequences, contributing to weight gain being one of them. Having a bihormonal glucose-control system that infuses a counter-regulatory agent (e.g., Glucagon) to reduce the frequency, extent, and duration of hypoglycemia can significantly reduce the amount of oral carbohydrates that are needed “medicinally” to treat or prevent hypoglycemia.

Certain embodiments of the present disclosure relate to a method for translating an amount of online counter-regulatory dosing (e.g. glucagon) computed by an autonomous glucose-control system to an amount of carbohydrates that the user is estimated to have been spared from needing by virtue of the counter-regulatory dosing, or that the user would be spaced from needing if the user had access to the counter-regulatory agent. In a bihormonal autonomous glucose-control system that infuses both insulin and a counter-regulatory agent/hormone, the method may include a mapping between the online counter-regulatory dosing, which was delivered to treat or prevent low glucose levels, and oral carbohydrates that are estimated to have otherwise been required to achieve a comparable safe control situation (had the counter-regulatory dosing not been delivered). In an insulin-only autonomous glucose-control system, where doses of a counter-regulatory agent/hormone are not delivered, but are still computed online, the method may include a mapping between the computed online counter-regulatory dosing and an estimated amount of oral carbohydrates that the subject will likely have been spared from needing to consume to treat or prevent low glucose levels had the counter-regulatory agent been available and its doses actually delivered.

Without loss of generality, embodiments disclosed herein include an autonomous glucose-control system where the counter-regulatory agent is glucagon. However, other medicaments and/or counter-regulatory agents may be utilized. The method may include relating computed online glucagon dosing with consumed oral carbohydrates for the treatment or prevention of low glucose levels (“treatment carbs”) as observed in real use (e.g., during clinical studies) in the insulin-only configuration, and relating the relationship between the counter-regulatory agent and carbohydrates to a similar relationship between delivered online glucagon doses (or other counter-regulatory agent) and similarly consumed oral carbohydrates in the bihormonal (insulin-glucagon) configuration.

Using data gathered from real use (e.g., clinical studies), a relationship between the consumed treatment carbs in an insulin-only configuration, Cᵢₒ, and the online computed (but not delivered) glucagon dosing, Gc, can be described by the relationship Cᵢₒ=Rᵢₒ(x)*Gc, where Rᵢₒ(x) may be a relating factor that can be a function of several dependencies that are included in vector x. Such dependencies can include the specific insulin and/or glucagon being used (e.g., their clinical properties), and/or the pharmacokinetic settings assumed by the control system in relation to insulin and/or glucagon. The dependencies can also include the user's body mass and the glucose target used by the glucose-control system. In some embodiments, Rᵢₒ(x) may be a constant, or Rᵢₒ(x)=Rᵢₒ, for a system exhibiting limited variation in the relationship between Cᵢₒ and G_{c} (e.g., due to limited effect, or limited or no variation in the associated dependencies).

Similar to the insulin-only configuration, from real-use data, a relationship between the consumed treatment carbs in a bihormonal (insulin-glucagon) configuration, C_{bh}, and the online delivered glucagon dosing, G_{d}, can be described by the relationship C_{bh}=R_{bh}(X)*G_{d}, where R_{bh}(x) may be described in a similar fashion to Rᵢₒ(x) above. In some cases, the quantities Cᵢₒ, G_{c}, C_{bh}, and G_{d} can refer to daily amounts, as averaged over some period of use (e.g., a week). In some cases, the quantities Cᵢₒ, G_{c}, C_{bh}, and G_{d} can refer to average daily amounts per body mass of the user, in which case dependency on body mass can be eliminated from x.

In cases where G_{c} is computed, but no glucagon is actually delivered in an insulin-only system, G_{c} has no effect on glucose insofar as treating or preventing low glucose levels, which in turn is generally expected to invoke further computed glucagon dosing (e.g., goes towards increasing the magnitude of G_{d} for a given situation). By contrast, since G_{d} is delivered in a bihormonal system, it is expected to have an effect in preventing or reducing the frequency, extent, or duration of low glucose levels, which in turn is expected to limit the overall magnitude of glucagon dosing (e.g., limits G_{d} for a given situation). As such, for a given set of dependencies, it is generally expected that G_{c}>G_{d} between the two system configurations. Likewise, since G_{c} has no effect in combating low glucose levels while G_{d} does have such an effect, it is expected that treatment carbohydrates Cᵢₒ>C_{bh}, when comparing the two system configurations.

If one can ideally relate, for a given real-use case of an insulin-only system with G_{c}, what the corresponding Cᵢₒ would have been for the same real-use scenario, had the computed online glucagon dosing actually been delivered as G_{d}, one can project an estimate that the user would have required “Cᵢₒ-C_{bh}” less treatment carbs (e.g., would have saved that much), had they instead been using a bihormonal system (with the same insulin controller), where glucagon would have been delivered. Conversely, if one can ideally relate, for a given real-use case of a bihormonal system with G_{d}, what the corresponding C_{bh} would have been for the same real-use scenario, had the delivered online glucagon dosing not been delivered but only computed as G_{c}, one can project an estimate that the user had actually avoided the need to take “Cᵢₒ-C_{bh}” additional treatment carbs, had they been instead using an insulin-only system (with the same insulin controller), where glucagon would not have been delivered. It should be understood that the above calculations are an estimate in an ideal situation as, in practice, it is not possible to have a re-run of a past real-use scenario to obtain such ideal relationships.

For practical implementation, real-use cases where the insulin-only system is used can be re-simulated while assuming a bihormonal system is available, where glucagon is assumed to be delivered. Since the control system may take delivered doses into account when issuing subsequent nearby glucagon doses, the simulated glucagon dosing may exhibit a reduction relative to the original G_{c} of the insulin-only system. With the glucose profile kept unaltered in a simulation, the simulation may lack reflecting any resulting glucose excursions in response to the assumed delivered glucagon dosing. The simulation in turn may lack reflecting the full reduction in glucagon dosing down to G_{d} that may have been observed if the glucose excursions had in fact benefited from glucagon being delivered. Thus, the reduced glucagon dosing that is observed in the simulation, pseudo delivered glucagon Ĝ_{d}, may arguably be exaggerated in magnitude relative to what would have been the “real G_{d}”. As described above, based on prior analyses G_{c} can be mapped to a corresponding amount Cᵢₒ in the insulin-only configuration, and Ĝ_{d} can be mapped to a corresponding amount Ĉ_{bh} in the bihormonal configuration. The simulation results, therefore, can map the reduction “G_{c}-Ĝ_{d}” to an estimate “CᵢₒĈ_{bh}” of treatment carbs that the user would spare had they been using the bihormonal system. The estimates may be conservative estimates. Repeating the simulation analyses across a variety of real-use cases that span the range of G_{c} observed in practice provides a global mapping between them and the associated range of (in some cases, conservative) estimates “Cᵢₒ-Ĉ_{bh}” of treatment carbs that the user would likely not need to consume had they been using the bihormonal system. Conversely, the mapping can be utilized when a bihormonal system is being used, where the observed dosing G_{d} is mapped back to a pseudo glucagon Ĝ_{c} and the resulting associated difference “Ĉᵢₒ-C_{bh}” provides a (in some cases, conservative) estimate of the treatment carbs that the user had likely saved by virtue of being on the bihormonal system.

Certain embodiments includes a system that comprises a controller for automatic control of a blood glucose level of a subject. The controller may be operative to generate an insulin dose control signal based on time-varying glucose levels of the subject as represented by a glucose level signal over time. The glucose level signal can be generated by a glucose sensor operative to continually sense a glucose level of the subject. The insulin dose control signal may control the delivery of doses of insulin by a delivery device. Further, the controller can operate at a regular frequency to generate an insulin dose control signal to regulate the glucose levels in the subject. During online operation, the controller can employ a control algorithm that generates a glucagon dosing signal, which may be mapped to an associated amount of oral carbohydrates.

The oral carbohydrates may be associated with the prevention or treatment of low glucose levels. Further, the mapping between the glucagon dosing signal and the oral carbohydrates may be derived from analysis of clinical data. The glucagon dosing signal may be computed, but not delivered in an insulin-only system configuration. In contrast, the glucagon dosing signal can be computed, and glucagon can be delivered in an insulin-glucagon system configuration. The computed glucagon dosing in an insulin-only system configuration can be mapped to an amount of oral carbohydrates that is estimated to have been saved had glucagon dosing been delivered if an insulin-glucagon system configuration had instead been used. The delivered glucagon dosing in an insulin-glucagon system configuration can be mapped to an amount of oral carbohydrates that is estimated to have been saved if an insulin-only system configuration had instead been used. The mapping may be dependent on the clinical properties of the insulin and glucagon being used, and settings in the control system related to the action and effect of insulin and glucagon. Further, the mapping may be dependent on the subject's body mass.

### Example Backup Therapy Reports

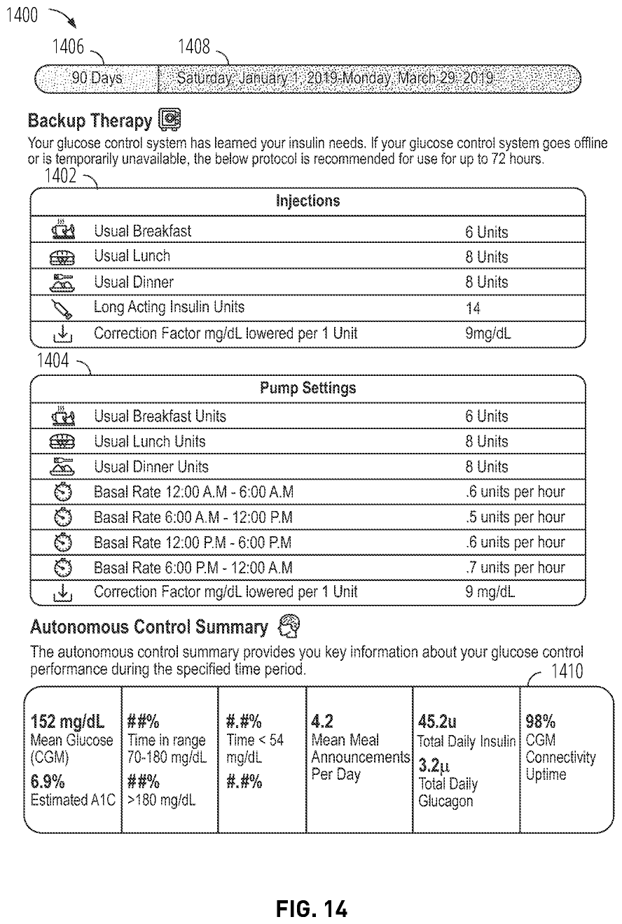

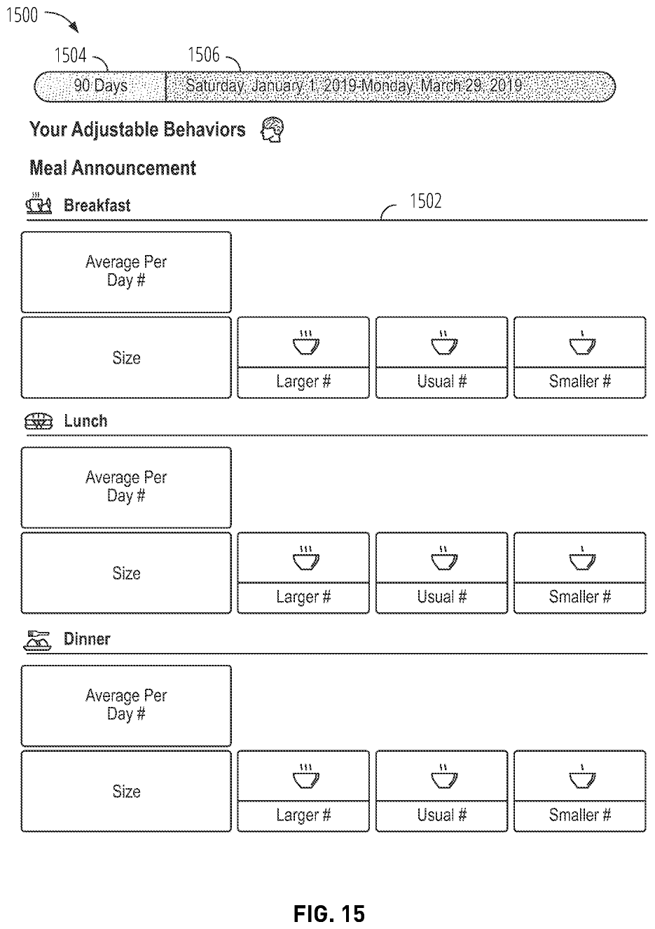

FIG. 14-FIG. 15 illustrate one non-limiting example of a backup therapy report, or a set of reports, that may be generated using one or more of the embodiments disclosed herein. In other words, the reports of FIG. 14-FIG. 15 may be portions of a single report generated by the glucose level control system 1102, or may be separate reports that are concurrently generated or that are generated based on different data and/or over different tracking periods. The report may be generated by the automated glucose level control system 1102, or by another computing system that may receive therapy data from the automated glucose level control system. Further, FIG. 14-FIG. 15 represent just one non-limiting example of a report or set of reports that may be generated. It is possible for other reports to be generated that include more or less data. For example, the backup injection therapy protocol and the backup pump therapy protocol illustrated in FIG. 14 may be separated into two separate reports that may be separately generated and/or accessed.

FIG. 14 illustrates a backup therapy protocol report 1400 in accordance with certain embodiments. The amount of insulin recommended under different ties and/or conditions may be displayed in units. In some cases, the backup therapy protocol report 1400 may identify the quantity of insulin included in a unit and/or the type of insulin. Further, in some cases, the backup therapy protocol report 1400 may be an interactive report that enables a user to modify a type of insulin or a unit size of insulin. In some such cases, the table 1402 may update the recommended number of units of insulin to administer under particular times or conditions based on the type of insulin and or unit size of insulin selected.

The backup therapy protocol report 1400 may identify the length of the tracking period 1406 used to determine the backup therapy protocol. Further, the report 1400 may identify the time or date range 1408 during which the tracking period 1406 occurred. Advantageously, knowing the tracking period 1406 may help determine an amount of trust to place in the recommendations included in the backup therapy protocols. The longer the tracking period, the more likely that the recommendations are accurate. A shorter tracking period is more susceptible to less accurate recommendations because, for example, the tracking period may encompass more days that are outliers for the subject's typical condition or activity level. For example, a tracking period of one day that occurs on a day when a subject consumed larger than normal meals or exercised significantly more than normal may result in backup therapy recommendations that do not match the subject's typical lifestyle. Further, knowing when the tracking period occurred may be useful to determine how current the recommendations are and whether they are a reliable indicator of an amount of insulin a subject should administer. For example, if the date range 1408 of the tracking period 1406 is a year old, and the subject has gained or lost significant weight over the year, the backup therapy protocol may no longer be a reliable indication of recommended injection therapy. In such cases, a user may adjust the recommendation and/or trigger a new occurrence of the backup therapy protocol generation process.

The table 1402 illustrates an example backup injection therapy protocol, which may indicate various insulin doses that may be administered to the subject 1104 at various times or under various conditions using injection therapy. The table 1402 identifies an amount of insulin the subject 1104 may inject when consuming a usual-sized meal for breakfast, lunch, or dinner. The usual-sized meal may refer to the size of a meal that the particular subject 1104 usually consumes or has been advised to consume by a healthcare provider. The units of insulin specified may refer to an amount of insulin that the automated glucose level control system 1102 provides the subject 1104 on average when the user consumes the identified usual size meal. In some cases, the table 1402 may further include recommended insulin doses for different size meals. For example, each breakfast may illustrate three different values (e.g., 5 units, 6 units, and 8 units) corresponding to light or smaller than usual breakfast, usual size breakfast, and heavy or larger than usual size breakfast.

It should be understood that the amount of insulin delivered may vary over time and/or based on the condition of the patient at a particular time. Thus, as indicated at the top of the report 1400, the recommendations in the backup therapy protocols are suggested for temporary use for a particular quantity of time (e.g., up to 72 hours in the illustrated example). The quantity of time for which the recommendations are valid may vary based on the subject 1104, the amount of historical data collected (e.g., the size of the tracking period), the amount of daily variation in the subject's blood glucose level, or any number of other factors that may affect the amount of time that the backup therapy protocol can be safely followed.

As illustrated by table 1402, the backup injection therapy protocol may further identify an amount of long-lasting insulin a subject 1104 is recommended to administer each day (or at certain times throughout the day). This long-lasting insulin may be used in place of the basal insulin that the glucose level control system 1102 may provide on a periodic basis.

In addition, the table 1402 identifies the reduction in glucose level attributable to one unit of insulin. For example, as illustrated in the table 1402, the automated glucose level control system 1102 has determined that one unit of insulin (e.g., 1/100^{th} of a milliliter of insulin) may reduce the blood glucose level of the subject 1104 by 9 mg/dL. Accordingly, a user implementing injection therapy may measure a blood glucose level of the subject 1104, determine a difference between the measured blood glucose level and a desired setpoint or threshold glucose level, and divide the difference by 9 to determine a number of units of insulin to inject in response to a determination that a correction dose is warranted (e.g., that blood glucose is outside of a desired setpoint range).

The table 1404 of the report 1400 provides an example of a backup pump therapy protocol. As illustrated, the backup pump therapy protocol may have the same therapy information as the backup injection therapy protocol for mealtimes and for the correction factor. However, because a pump may be capable of providing periodic basal therapy, the long acting insulin units of the injection therapy may be replaced with a basal rate indicating a rate at which the backup or replacement pump should administer insulin to the subject. As illustrated, the basal rate may vary over time. In the illustrated example, a basal rate is supplied for four different time periods constituting a 24-hour day. However, the basal rate may be divided into a fewer (e.g., 2 twelve-hour blocks) or greater (e.g., every four hours) number of periods, with each time period potentially having a different basal rate as determined based on the historical therapy data provided by an automated glucose level control system.

In some cases, the report 1400 may include additional data that may be tracked over the tracking period. This additional data may include any data that may facilitate care of the subject 1104 and/or maintenance of the automated glucose level control system 1102. Some non-limiting examples of additional data that may be tracked and included in a report using, for example, a backup therapy process or therapy control process are illustrated in chart 1410 of the report 1400. For example, as illustrated in the chart 1410, the report may include the average blood glucose level of the subject 1104 over the tracking period and/or the corresponding estimated A1C percentage. Further, the report 1400 may indicate the amount or percentage of time that the subject's blood glucose level is within a desired setpoint range and/or is above the desired setpoint range. Similarly, the report 1400 may indicate the amount or percentage of time that the subject's blood glucose level is below a threshold blood glucose level.

In addition, the report 1400 may indicate the average number of meal announcements per day. As illustrated in the chart 1410, the subject 1104 from which the example report 1400 was generated made an average of 4.2 meal announcements indicating that on average, the subject consumed more than 3 meals a day. In some cases, the report may further indicate the types of meals announced (e.g., two breakfasts, one lunch, and one dinner). The second breakfast may be a large snack that is roughly equivalent in size to a small breakfast for the subject. Thus, the subject may have made an additional breakfast meal announcement. In some cases, the automated glucose level control system 1102 may support a separate snack or other meal announcement option.

The report 1400 may further include the total amount of insulin administered to the subject per day, and/or the total amount of counter-regulatory agent (e.g., glucagon) administered to the subject per day. In addition, the report 1400 may indicate the amount of percentage of time that the automated glucose level control system 1102 is able to connect or communicate with the CGM sensor over the tracking period, which may correspond to the amount of time that the automated glucose level control system 1102 functions in an online mode during the tracking period.

FIG. 15 illustrates a meal selection report 1500 that may include a table 1502 identifying the average number of times per day that a user (e.g., the subject 1104) announces each meal type. Typically, a user will announce a meal 0 or 1 times a day. However, in some cases, a user may announce a particular mealtime more than 1 time to account, for example, for large snacks that may be similar in size to a particular meal. Smaller snacks often may be handled by the control algorithm of the automated glucose level control system 1102 (e.g., by the corrective insulin controller 1208) without a meal announcement.

Further, the table 1502 may identify the number of times over the tracking period, or selected time period within the tracking period, that meals of particular sizes are announced by a user. For example, the table 1502 may indicate the number of times that a usual size meal is announced, a smaller than usual size meal is announce, or a larger than usual size meal is announced.

### Automated Glucose Control Refinement

An ambulatory medical device (AMD) may include a control system that autonomously provides therapy to a subject, for example, based on a health condition of a subject (e.g., determined based on one or more measured physiological indicators or parameters of the subject). In some examples, the control system may determine the therapy time and/or the intensity of the therapy during each therapy delivery based on one or more measured physiological parameters (e.g., using one or more subject sensors, such as a CGM sensor) and according to a predictive model that may include one or more control parameters. In some examples, the predictive model may be used to estimate a physiological effect of the therapy in order to adjust the therapy delivery according to an intended physiological effect. It is desirable to adaptively adjust the values of the control parameters to optimize the therapy delivery to a subject in the presence of time varying and subject specific factors that may influence the physiological effects of a therapy delivery on the subject. In some cases, the AMD may be an ambulatory medicament device that regulates the level of an analyte in subject's blood. An example of such ambulatory medicament device is an automated glucose level control system (e.g., the glucose level control system 1102) that may automatically provide insulin and/or a counter-regulatory agent (e.g., Glucagon) to a subject 1104 to help control the blood glucose level (BGL) of the subject 1104. Generally, a control algorithm may be implemented by the automated blood level glucose level control system 1102 to determine when to deliver insulin and/or how much insulin to provide to the subject 1104. Further, the control algorithm may control both an ongoing or periodic delivery of insulin (e.g., a basal dose), and a correction bolus that may be provided to adjust a subject's blood glucose level to within a desired range. The control algorithm may use blood glucose level readings obtained from a subject sensor (e.g., a sensor measuring one or more physiological parameters of the subject in real time), such as a continuous glucose monitoring (CGM) sensor, that obtains automated blood glucose measurements from the subject. Moreover, in some cases, the control algorithm may deliver a bolus of insulin in response to an indication of a meal to be consumed or being consumed by the subject 1104.

Insulin may be administered subcutaneously into blood of a subject 1104. For example, the glucose level control system may subcutaneously deliver a medicament (e.g., insulin, glucagon) via an infusion set connected to a site on subject's body. There is often a delay, referred to as pharmacokinetic (PK) delay, between when the insulin is provided and when the amount of insulin in the subject's blood plasma reaches a particular concentration level, such as maximum concentration. This amount of time may vary based on the type of insulin and/or on the physiology of the particular subject. For example, with a fast-acting insulin, it may take approximately 65 minutes for a bolus of insulin to reach maximum concentration in the blood plasma of one subject, but 60, 64, or 70 minutes for another subject. For some other types of insulin, it may take anywhere from 3-5 hours to reach maximum concentration in the blood plasma of the subject. Additionally, there might be a delay, referred to as pharmacodynamic (PD) delay, between variation of the amount of insulin in the subject's blood plasma and the resulting variation of glucose level in the subject's blood. In some examples, the value of pharmacodynamic (PD) delay may be used to estimate BGL based on an estimated concertation of insulin in patient's blood.

In some cases, the glucose level control system may implement a predictive algorithm based on a pharmacokinetic (PK) model to estimate the accumulation of insulin in the blood plasma of the subject over time, following the subcutaneous administration of insulin to a subject. In some examples, the PK delay may be subject specific and/or change overtime. Accordingly, in these examples, the PK model may include one or more parameters, referred to as control parameters, that may be subject specific and/or change overtime. Examples of factors and parameters that may influence the PK delay and/or the control parameters of the PK model may include, type of insulin, blood glucose level (e.g., at the insulin administration time), physiological characteristics of the subject, health condition of the subject, one or more physiological parameters of the subject, time of the administration, location at which the infusion set is placed, the amount of insulin administered and the like. The physiological characteristics may include characteristics shared among large portions of the population (e.g., weight, gender, age, etc.) as well as characteristics that may be unique or specific to the subject, or shared among few people (e.g., characteristics related to genetics). Differences between the physiologies of different subjects may result in differences in the optimal blood glucose range for each subject, or some subset of subjects. Further, the differences in physiologies may also affect the absorption of insulin into the blood plasma. In other words, different physiologies of different subjects may result in insulin absorption taking different amounts of time for different subjects. Thus, while the maximum concentration of glucose in blood plasma may occur 65 minutes after delivery of a bolus of fast-acting insulin for one subject, it may be 60 minutes or 70 minutes for another subject.

Accordingly, in some such examples, the blood glucose level control system 1102 (e.g., the glucose level control system of an AMD) may implement a method to adaptively change the one or more control parameters in of the PK model used in its control algorithm to modify its predictions, in order to maintain the BGL within a desired range. For example, the glucose level control system may use readings from one or more subject sensors (e.g., a CGM) and/or information received from the subject (e.g., using a user interface of the AMD), to modify one or more control parameters.

As indicated above, a blood glucose system, such as an automated blood glucose level control system 1102, may control delivery or administering of insulin, or a counter-regulatory agent, based on a PK model and one or more blood glucose level measurements of the subject. In some examples, the PK model can be a bi-exponential PK model that may be used to estimate or determine the absorption or accumulation of subcutaneously administered insulin into blood and/or a decay rate of the insulin level in the subject's blood for a given value of delivered dose of insulin. In some examples, the absorption of insulin over time according to a bi-exponential PK model may be represented by the following equation:

p(t)—KU₀(e^{-α}^{₁}^{t}−e^{-α}^{₂}^{t}) (2)

where U₀ is the subcutaneous dose in units (U), K is a scaling constant, and α₁ and α₂ are time constants that may be used as the control parameters of the model. In some examples, the peak time of absorption of insulin, starting from the time that subcutaneous dose (U₀) is administered, may be referred to as Tmax and can be determined based on the following equation:

$$log log \frac{( \frac{{α}_{2}}{{α}_{1}} )}{( {α}_{2} - {α}_{1} )} \qquad ( 3 )$$

In some examples, α₁ and α₂ can be related (e.g., through an equation such as α₂=1.5 α₁ or any other linear or nonlinear mathematical relations). In some such examples, Tmax alone may be used as the control parameter of the bi-exponential PK model. In some cases, Tmax may be referred to the time at which the concentration of insulin in subject's blood reaches a maximum level (e.g., starting from the time that subcutaneous dose is administered). In some other examples, the bi-exponential PK model may be used to estimate or determine the accumulation of counter-regulatory agent or hormone (e.g., glucagon) in subject's blood. Equation 2 may be used to calculate the pending effect of the accumulated amount of insulin in the subcutaneously administered dose, as that can be taken to be the difference between the total area (∫₀^{∞}p(t)dt, which can describe a measure of the total amount of hormone (e.g., insulin) that can be absorbed due to a dose U₀) and ∫₀^{t}p(t)dt, which can represent a measure of the expended portion of U₀ at time t.

Often, the glucose level control system is configured to maintain a subject's blood glucose within a particular range (e.g., a normal range). As blood glucose rises or falls, the glucose level control system may administer particular amounts of insulin or counter-regulatory agent to the subject to bring the blood glucose level of the subject back to within a desired range or closer to a desired setpoint. As explained above, it may take some non-infinitesimal amount of time for the medicament to be absorbed into the subject's blood stream. Thus, a PK model (e.g., the bi-exponential PK model), may be used to determine how much insulin or counter-regulatory agent should be provided to the subject in order to maintain the subject's blood glucose within a particular range. In some examples, the PK model (e.g., the bi-exponential PK model) may be used to predict the concentration of insulin blood glucose level of the subject over time as insulin or counter-regulatory agent is administered. In some cases, the control parameter values of the PK model may be set by a healthcare provider based on default values obtained through clinical trials and/or based an individualized treatment plan for the subject as may be determined based on clinical tests of the subject and/or on the healthcare provider's evaluation of the subject, which may be determined based on tests of the subject.

However, as previously indicated, the pharmacokinetic delay and the control parameters of the PK model, may be subject specific and/or change overtime due to various factors. Thus, although clinical data may determine optimal or recommended values of the control parameters for an average subject through one or more trials, the determined data may not be optimal for a particular subject. Moreover, individualized treatment plans are typically based on point-in-time measurements. These point-in-time measurements may provide a good guideline for treatment, but the optimal values of the control parameters for a subject may vary at different times of day, due to different activities, due to changes in the subject over his or her lifetime, or for any other number of reasons.

The glucose level control system 1102 of the present disclosure can implement a method or process to autonomously and/or automatically modify one or more control parameters of a control algorithm, or the model used by the control algorithm, to modify therapy provided to the subject using the glucose level control system 1102. The method may be performed by a hardware separate processor 1120 and/or a controller 1110 that controls the administering of therapy. The system can provide therapy (e.g., insulin) to a subject in response to a determination of a blood glucose level of the subject. The blood glucose level may be determined based at least in part on a glucose level signal obtained from a glucose level sensor that is operatively connected to a subject. The determination of the therapy (e.g., an amount of insulin or counter-regulatory agent) may be based at least in part on the blood glucose level and/or the bi-exponential model. Moreover, the determination of therapy may be based at least in part on a value or setting of one or more control parameters of the glucose level control system. The one or more control parameters may be, or may correspond to, one or more parameters of the bi-exponential PK model, or any other model or control algorithm used to control the administering of therapy to the subject.

As mentioned above, the glucose level control system 1102 may provide the therapy based on the value or setting of the one or more control parameters. The value or setting of the one or more control parameters may be based on an initial configuration of the glucose level control system 1102 by a healthcare provider, subject, or other user. Further, the initial configuration may be based on clinical data or data obtained that is specific to the subject. In some cases, a control parameter may be a time constant used by a control algorithm of the glucose level control system (e.g., Tmax in a bi-exponential PK model). This time constant may be used in a calculation of an accumulation of insulin in the subject by the control algorithm. Further, the control parameter may be used to control an insulin dosing response of the control algorithm to a blood glucose excursion in the subject as indicated by a glucose level signal obtained from a glucose level sensor. In some cases, the control parameter may be, or may be related to, Tmax (e.g., defined by equation 2). For example, the control parameter may be an estimate of Tmax or a fraction (e.g., 0.5) of Tmax. As previously explained, Tmax may be the peak time of absorption of insulin, or the amount of time until the concentration of insulin from an insulin dose reaches maximum concentration in the blood of the subject.

Moreover, the control parameter may be associated with a setpoint or target blood glucose level, or a blood glucose range. For example, the control parameter could relate to a point in time when an estimated amount of “insulin on board” (e.g., an amount of insulin in the subject as determined by a model of insulin accumulation and/or utilization in the subject) falls below a threshold value. As another example, the control parameter can be a clearance time for insulin boluses (e.g., an estimate of an amount of time for an administered bolus of insulin to be utilized by the subject). In some cases, the control parameter may relate to T1/2, which corresponds to a time when the concentration of insulin in the blood plasma reaches half of the maximum concentration in the blood plasma. In some cases, the control parameter may be a parameter that can be used to calculate Tmax or T1/2.

In some examples, the glucose level control system 1102 may determine an effect of the supplied therapy (herein referred to as therapy effect or effect). For example, the therapy effect may be determined by analyzing a glycemic control of blood glucose (e.g., variation of BGL or supplied therapy over a measurement period) in the subject's blood as indicated by the glucose level signal received from the glucose sensor (e.g., a CGM sensor). In some cases, the control system may measure or determine the effect of the supplied therapy over time. In some such cases, the therapy effect may be determined based on variation of BGL and/or the amount of therapy delivered over time. Moreover, in some cases, the system may continue to supply therapy to the subject over several therapy delivery times or instances and may average or otherwise aggregate the measured or determined effects of the therapy over the several therapy delivery times or instances. In some other examples, the glucose level control system 1102 may determine the therapy effect based at least in part on an input received from the subject. The input received from the subject may include a subjective or objective effect. The input received from the subject may include manual blood glucose level measurements obtained using, for example, test strips. Another example of input may be an indication of light-headedness, difficulty breathing, headaches, or any other objective or subjective effect identified by the subject.

Based at least in part on the provided therapy and the measured or determined effects of the therapy (e.g., the changes in blood glucose level attributed to the therapy), the glucose level control system 1102 may autonomously determine a modification to one or more control parameters. For example, the control system may modify Tmax value used by the control algorithm (or the PK model used in the control algorithm), for example, to improve the effect of a subsequent therapy that may be provided to the subject. As such, the directional modification (e.g., increase or decrease) of a control parameter value may depend on the measured or determined effect of the therapy provided based on the initial or prior value of a control parameter. Moreover, the directional modification of the control parameter value may depend on a difference between the determined or measured effect of the blood glucose therapy and an expected effect of the blood glucose therapy (e.g., calculated based on PK model). In some examples, the directional modification of a control parameter may be determined based on the amount of therapy doses provided and/or measured BGL of the subject, during and between one or more previous therapy deliveries.

In some examples, the pharmacodynamic delay for a subject may be a known value. In these examples, the amount of absorbed insulin in the subject's blood may be estimated based on the measured value of BGL received from a glucose sensor. In some such examples, the directional modification may depend on the difference between calculated value of absorbed insulin based on a PK model (e.g., bi-exponential PK model) with a selected value of Tmax, and the estimated value of the absorbed insulin based on the measured value of BGL received from a glucose sensor.

Using the modified control parameter, the glucose level control system 1102 may determine therapy to deliver to the subject 1104 at a therapy delivery time. As with the initial control parameter, therapy may be delivered during one or more therapy delivery times based on the modified control parameter. The system may determine the effect of the therapy delivered based on the modified control parameter using one or more of the embodiments previously described with respect to the therapy delivered using the initial control parameter.

In some examples, the control system can compare the measured, determined or reported effects (e.g., physiological effects) from the therapy delivered using the initial value of a control parameter and those from the therapy delivered using the modified value of the control parameter. Based on the comparison, the control system may determine which values of the control parameter is preferable for the subject. In some examples, the comparison may be performed in real-time, or substantially in real-time. Further, the comparison may be performed by the glucose level control system 1102 without user interaction. The comparison may be performed using a comparison method and based on one or more comparison criteria.

The comparison method may be based on finite number of therapy effects determined or measured at discrete times or based on continuous temporal variations of an effect over a period. In some examples the comparison method may involve statistical analysis of the measured or determined effects resulting from usage of the initial value and modified value of the control parameter. The comparison criterion may be based on the effects or based on the temporal variations of the effects over a period. For example, the preferable control parameter value can be a value that causes the blood glucose level of the subject to stay within a desired range or closer to a setpoint level for the subject. Accordingly, the system can set or maintain the control parameter to have the value that generated blood glucose levels that are closer to the desired range or setpoint for the subject for subsequent therapy.

In some cases, the glucose level control system 1102 may repeat the process for different control parameter values enabling the system to refine the glucose control for the subject over time. In subsequent performances of the process, the initial control parameter value may not be an initial value but may be the most recent selected value for the control parameter based on the determined effects of the control parameter.

In some cases, the determination of a second or modified value for a control parameter, or the modification of the control parameter may be triggered based on a glucose level of the subject not satisfying a threshold. Alternatively, or in addition, a process of modifying a control parameter value may be triggered based on a difference between an expected glucose value of a subject and an expected glucose value of a subject after the administering of therapy exceeding a threshold.

Using the embodiments described herein, the value of a control parameter may be autonomously modified without interaction by a subject or user with the glucose level control system. In other words, the glucose level control system can automatically adjust and/or refine a control parameter used by a control algorithm for glycemic control of the subject.

As previously described, the glucose level control system may provide both insulin therapy and counter-regulatory agent therapy to a subject. In some cases, the glucose level control system may only provide insulin therapy. In some such cases, the glucose level control system 1102 may output an indication of an amount of counter-regulatory agent that may or should be administered to the subject based on a detected condition of the subject.

The active control parameter value used by the control parameter may remain active until a subsequent occurrence of the therapy modification process. In some cases, performance of the therapy modification process is continuously performed with the control parameter value being modified based at least in part on a determined effect of the prior control parameter value. In other cases, the therapy modification process is performed until the determined effect of the therapy satisfies a desired threshold (e.g., when the detected blood glucose level is within a threshold of a setpoint or median setpoint value). In some cases, the therapy modification process is performed a set amount of times and the control parameter value that provides the best outcome (e.g., closes to desired blood glucose level) is set as the active control parameter for subsequent therapy. In some cases, providing therapy at different sites on the subject's body (e.g., back, stomach, leg, or arm) may result in different blood glucose absorption rates (associated with different PK delays). Thus, in some such cases, the therapy modification process may be performed each time the infusion set used to deliver the therapy is moved to a different site on the subject.

### Example Automated Glucose Control Refinement Process

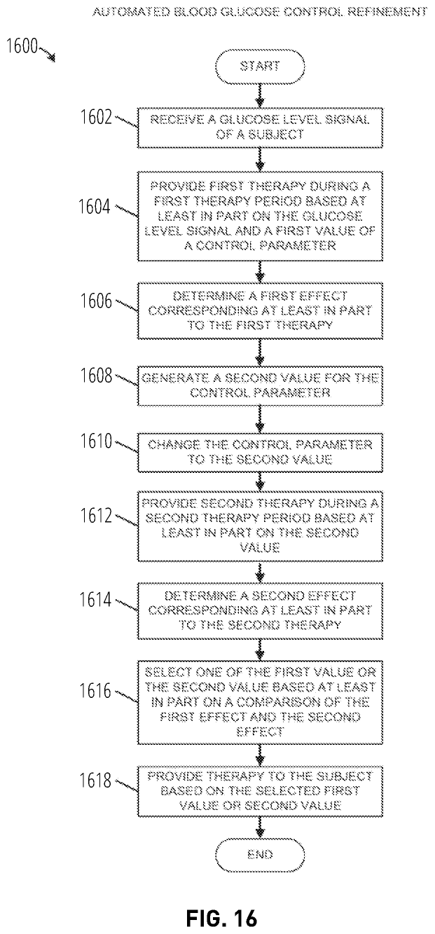

FIG. 16 presents a flowchart of an example automated glucose control refinement process 1600 in accordance with certain embodiments. The process 1600 may be performed by any system that can autonomously and/or automatically modify a control algorithm and/or a control parameter that affects execution of the control algorithm based on feedback (e.g., from a blood glucose signal) relating to therapy administered to a subject 1104. For example, the process 1600 may be performed by one or more elements of the glucose level control system 1102. In some cases, at least certain operations of the process 1600 may be performed by a separate computing system that receives blood glucose data from the glucose level control system 1102. Although one or more different systems may perform one or more operations of the process 1600, to simplify discussions and not to limit the present disclosure, the process 1600 is described with respect to particular systems.

The process 1600 may be performed automatically and without user interaction. In some cases, a user may trigger the process 1600 via a command or interaction with a user interface. However, once the process 1600 is triggered, the process 1600 may be performed automatically. Further, the process 1600 may be performed continuously, periodically, or in response to a trigger. The trigger may be time based and/or based on a measurement of the glucose level of the subject. For example, the trigger may correspond to a determination that a glucose level of a subject differs by more than a threshold from a predicted glucose level that is predicted by a glucose level control algorithm based on the administering of medicament. Further, the trigger may be based on the activation or first time use of the glucose level control system 1102 by the subject 1104.

The process 1600 begins at block 1602 where the glucose level control system 1102 receives a glucose level signal corresponding to the glucose level of a subject 1104. The glucose level signal may be received from a glucose sensor capable of measuring the level of glucose in the blood of the subject. For example, the sensor may be a continuous glucose monitoring (CGM) sensor.

At block 1604, the glucose level control system 1102 provides a first therapy during a first therapy period to the subject 1104. The first therapy and/or first therapy period may be based at least in part on the glucose level signal and a first value of a control parameter. The control parameter may include any control parameter that affects operation of the glucose level control system 1102 and/or performance of a control algorithm of the glucose level control system 1102. The control algorithm may include any control algorithm used to determine a dose of medicament (e.g., insulin) to administer to the subject 1104. In other words, the controller 1110 or the separate processor 1120 may use the control algorithm to generate a dose control signal based at least in part on a value (e.g., the first value of the block 1604) of the control parameter to cause the delivery device(s) 1106 to administer a dose of insulin or other medicament.

In some cases, the control algorithm may be based on the PK model (equation 2). Further, in some cases, the control parameter may be Tmax, which may be calculated using equation 3. In other cases, the control parameter may be T_{1/2}, which may relate to the amount of time for the dose of insulin in the blood stream to drop to ½ of the maximum concentration in the blood attributable to the dose administered to the subject 1104. In some cases, the control parameter corresponds to a time until insulin within blood plasma of the subject reaches a particular concentration level subsequent to administration of an insulin dose. Moreover, in some cases, the control parameter may be a parameter that affects the determination of Tmax, such as one or more of the time constants α1 and α2. In some implementations, the control parameter may be used by the control algorithm to account for and/or determine an accumulation of insulin (or other medicament) in the subject 1104 and/or a rate of diminishment of the insulin (or other medicament) in the subject 1104. In some cases, the control parameter may be used to control an insulin dosing response of the control algorithm to a blood glucose excursion in the subject as indicated by the glucose level signal received at the block 1602.

In some instances, the control parameter may relate to at least one time constant used in a calculation of an accumulation of insulin in the subject by the control algorithm, such as one or more of the time constants α₁ and α₂ that may be used in the calculation of Tmax. In some cases, the control parameter may correspond to a rate of insulin diminishment in the subject 1104. In some cases, the control parameter may relate to a target setpoint or a target setpoint range for maintaining or attempting to maintain the blood glucose level of the subject 1104.

The first therapy may correspond to a single administering of insulin to the subject 1104. This single administering of insulin may be any type of insulin administered for any reason. For example, the insulin dose may be a basal insulin dose, a priming dose, a dose supplied in response to a meal announcement, or a correction dose of insulin. Moreover, the first therapy may be medicament other than insulin, such as counter-regulatory agent (e.g., glucagon). In some cases, the first therapy may be a plurality of medicament (e.g., insulin and/or counter-regulatory agent) doses supplied or administered to the subject 1104 over the first therapy period. Further, the plurality of medicament doses may include a variety of types of medicament doses, such as one or more basal doses, one or more meal doses associated with one or more meal announcements, one or more corrective doses, etc.

The first therapy period may be a time period that corresponds to a single medicament dose. Alternatively, the first therapy period may be a time period that encompasses a plurality of medicament doses. Further, the time first therapy period may be a time period associated with a defined length of time. Alternatively, or in addition, the first therapy period may be defined based on a number of medicament delivery periods. In other words, the time period may vary based on the amount of time it takes to deliver or administer a specified number of doses of medicament (of any type or of a particular type). The first therapy period may be between about 15-90 minutes, about 1-18 hours, about 1-3 weeks, about 1-6 months, or about 1-3 years as specified via user interaction

The first value may be selected based on a prior therapy or a prior performance of the process 1600. In some cases, the first value is selected based on a baseline value. The baseline value may be associated with clinical data, or it may be determined based on initial operation of the glucose level control system 1102 for some period of time before performance of the process 1600. Alternatively, or in addition, the first value may be selected based on clinical data or a particular prescription for the subject 1104. In some cases, the first value may be based on clinical data for average users or average users that share certain physiological data with the subject 1104. In some cases, the first value is determined based on a healthcare provider's assessment of the subject 1104. Further, the first value may be determined based on an infusion site (e.g., back, stomach, leg, etc.) for the glucose level control system 1102. In some cases, the first value may be selected based on demographics or characteristics of the subject 1104. For example, the first value may be based on the gender, weight, body mass, or age of the subject 1104.

At block 1606, the glucose level control system 1102 determines a first effect corresponding, or attributable, at least in part to the first therapy. Determining the first effect may include receiving a glucose level signal from the glucose level sensor operatively connected to the subject. This glucose level signal may be a subsequent or updated glucose reading that is more recent than the glucose level signal received at the block 1602. The glucose level signal received at the block 1602 may be used to determine therapy to administer to the subject 1104 and the glucose level signal received at the block 1606 may be used to determine a result of the administered therapy. It should be understood that glucose level signals may be received continuously or periodically and can be used to both determine therapy to administer and to determine the effect of the administered therapy.

In some cases, determining the first effect may include analyzing glycemic control of blood glucose in the subject as indicated by the glucose level signal. Analyzing the glycemic control of the blood glucose in the subject may include tracking the blood glucose level of the subject 1104 over time. Further, analyzing the glycemic control of the blood glucose in the subject may include comparing the blood glucose level of the subject 1104 over time to a predicted blood glucose for the subject 1104 over time as predicted based on the PK model used in the control algorithm using the selected value for the control parameter. As mentioned above, in some examples, the measured blood glucose level of the subject 1104 over time may be used to calculate the accumulation and/or diminishment of the insulin level in subject's blood. In these examples, analyzing the glycemic control of the blood glucose in the subject may include determining whether, or to what degree, the calculated accumulation and/or diminishment of insulin (or other medicament) using the PK model (e.g., bi-exponential PK model) and the control parameter values used in the control algorithm matches the accumulation or diminishment of insulin (or other medicament) estimated based on the measured blood glucose level (e.g., obtained from the CGM sensor). In some cases, the first effect may, at least partially, be determined by analyzing one or more signals received from one or more subject sensors that measure one or more physiological parameters of the subject (e.g., heart rate, temperature and the like).

In yet other examples, the first effect may be determined based on an input received from the subject (e.g., using a user interface of the AMD). In some cases, the first effect may be determined based at least in part on an assessment or input provided by the subject 1104 (e.g., using a user interface) with respect to the first value or the first effect. For example, if the subject 1104 feels woozy, dizzy, lightheaded, nauseous, or otherwise uncomfortable during the first therapy period, the subject 1104 may, via, for example, a touchscreen display of the AMD, indicate how the subject 1104 is feeling.

At block 1608, the glucose level control system 1102 obtains a second value for the control parameter. This second value may be autonomously determined. Further, in some cases, the second value may be automatically determined. In some cases, the second value is determined based at least in part on a user triggering the glucose control refinement process 1600. In some such cases the control system may determine the second value and present it to the user via a user interface 1124 of the glucose level control system 1102.

In some other examples, the second value may be obtained from a user interface 1124 of the glucose level control system 1102 (e.g., in response to a user interaction with the user interface). In some examples, the second value may be obtained from a computing system that is connected to or otherwise in communication with the glucose level control system. The communication connection may be a wired or wireless connection. Further, the wireless connection may be a direct connection (e.g., via Bluetooth or other near-field communication technologies) or a connection over a network (e.g., a local area network, a wide area network, a cellular network, etc.). The user interface 1124 can be implemented in the form of a graphical user-interface configured to output information and receive information via user interaction.

The second value may be an increase or decrease of the control parameter compared to the first value. The second value may be limited to a particular maximum change from the first value. Further, the second value may be selected based at least in part on the first effect. For example, if the first effect corresponding to the first value results in blood glucose being near an upper range of the setpoint range, the second value may be selected in an attempt to being the blood glucose level closer to the middle of the setpoint range. Further, the second value may be selected based at least in part on characteristics of the subject 1104, such as age, weight, gender, or any other characteristics that may affect blood glucose management. In some examples, the second value may be selected based at least in part on the first effect determined based on an assessment provided by the subject 1104, in an attempt to reduce the symptoms felt by the subject 1104.

In some cases, the second value of the control parameter may be generated based at least in part on a baseline value of the control parameter and an output of a function defined based on glycemic control of the subject. In some examples, the glycemic control of the subject may include the measured value of the glucose level in subject's blood (e.g., provided by the CGM) and/or the amount of therapy (e.g., dose of insulin or counter-regulatory hormone) provided during the first therapy period. The baseline value of the control parameter may correspond to the first value used to provide therapy at the block 1604. This baseline value may be a last known optimal value for the subject prior to any changes to the subject (e.g., change in weight, insulin type, or metabolism changes, etc.). Alternatively, or in addition, the baseline value may be a value determined by a healthcare provider. In some cases, the second value of the control parameter is based at least in part on glycemic control indicated by the glucose level signal.

In some cases, the second value may be a modification to Tmax or T_{1/2}. It should be understood that Tmax and/or T_{1/2} may, at least in part, be based on the physiology or biochemistry of the subject 1104. Thus, the setting of either Tmax or T_{1/2} or the setting of the first value and the second value may refer to setting a parameter of the control algorithm or the PK model used by the control algorithm, representative of or corresponding to Tmax and/or T_{1/2}. For example, the setting of the first value and the second value may include setting one or more control parameters that may be used to determined or estimate Tmax and/or T_{1/2} for the subject 1104. However, the set value may differ from the actual value of Tmax and/or T_{1/2} for the subject 1104. Further, as Tmax and/or T_{1/2} may vary for different subjects, it is not always possible to explicitly set or determine Tmax and/or T_{1/2} for a subject. Instead, Tmax and/or T_{1/2} may be estimated or determined by comparing the effects and/or blood glucose levels determined for different control parameter values that correspond, at least in part, to Tmax and/or T_{1/2} Using the process 1600, the control parameter may iteratively approach the actual Tmax and/or T_{1/2} for the subject 1104, or within a threshold of the actual Tmax and/or T_{1/2} for the subject 1104. Alternatively, using the process 1600, the control parameter (such as one or more of the time constants α₁ and α₂) may iteratively approach a value that corresponds to the actual Tmax and/or T_{1/2} for the subject 1104.

At block 1610, the glucose level control system 1102 changes the control parameter to the second value. Changing the control parameter to the second value causes a change in the operation or execution of the control algorithm. This change in the execution of the control algorithm may result in a change in one or more factors associated with the provisioning of therapy to the subject 1104. For example, the changing in the execution of the control algorithm may result in a change in an amount of medicament delivered, a timing of the delivery of the medicament, a rate at which a dose of medicament is delivered to the subject 1104, a target setpoint or target range for the blood glucose of the subject, a threshold used in determining whether to deliver medicament (e.g., a threshold difference from the target setpoint), or any other factor that may affect therapy delivered to the subject 1104.

At block 1612, the glucose level control system 1102 provides second therapy during a second therapy period to the subject 1104. The second therapy is based at least in part on the updated control parameter that is updated to the second value at the block 1610. As with the first therapy, the second therapy may refer to one or a plurality of medicament doses. Further, the second therapy period may refer to a specific amount of time, an amount of time to deliver a particular number of medicament doses, or a particular number of medicament doses. In some cases, the block 1612 may include one or more of the embodiments described with respect to the block 1604 but using the second value for the control parameter over the second therapy period. In some examples, the duration of the second therapy period may be equal to the duration of the first period. In some other examples, the number of therapies delivered during the second therapy period may be equal to the number of therapies delivered during the first second therapy period.

At block 1614, the glucose level control system 1102 determines a second effect corresponding at least in part to the second therapy. The block 1614 may include one or more of the embodiments described with respect to the block 1606, but with respect to the second therapy.

At block 1616, the glucose level control system 1102 selects one of the first value or the second value based at least in part on a comparison of the first effect and the second effect. The comparison of the first effect and the second effect may be performed autonomously without action by a user. The glucose level control system 1102 may select the one of the first value or the second value to be a current or active value for the control parameter based on whether the first effect or the second effect results in improved care (e.g., closer to a desired setpoint for a greater period of time, or less volatility in blood glucose values, or any other factor that a healthcare provider may use to evaluate the success of diabetes management) for the subject 1104. In some cases, the glucose level control system 1102 selects a third value to the current or active value for the control parameter. The third value may be selected based on the comparison of the first effect and the second effect. For example, if it is determined that the first effect is preferable to the second effect, the third value may be selected based on a change to the first value in the opposite direction as the change made to the first value to obtain the second value. For instance, if in the prior example, where it is determined that the first effect is preferable to the second effect, the first value corresponded to a Tmax of 60 minutes, and the second value was selected to correspond to a Tmax of a longer time period (e.g., 65 or 70 minutes), the third value may be selected to correspond to a Tmax of a shorter time period (e.g., 50 or 55 minutes).

Comparing the first effect and the second effect may include determining whether the first value or the second value brought the glucose level of the subject 1104 closer to a target setpoint and/or maintained the glucose level of the subject 1104 within a target range for a longer period of time. In some cases, comparing the first effect and the second effect may include determining whether the first value or the second value resulted in a more stable blood glucose level for the subject 1104 or less volatility in the blood glucose level of the subject 1104. In some cases, comparing the first effect and the second effect may include determining whether the first value or the second value resulted in more and/or greater excursions of the glucose level of the subject 1104 blood glucose level from a target blood glucose range.

Comparison of the first effect and the second effect may be performed in real-time or substantially in real-time accounting for the processing speed of the hardware separate processor 1120 or the glucose level control system 1102. Thus, in some cases, the comparison of the first effect and the second effect may be performed upon determination of the second effect.

In some embodiments, the comparison of the first effect and the second effect may include a statistical comparison or statistical analysis of the first effect and the second effect. In some cases, the comparison of the first and second effects may include determining whether the second therapy produced a statistically significant improvement in therapy (e.g., glycemic control) compared to the first therapy. A statistically significant improvement may vary depending on the subject or the condition of the subject. The comparison can also include a determination of whether there was a statistically significant increase in risk factors (e.g., hypoglycemia) during the second therapy period compared to the first therapy period. In some embodiments, a statistically significant improvement may be an improvement determined based on a first statistical analysis of a set of data associated with the first effect and a second statistical analysis associated with the second set of data associated with the second effect. For examples, the first and second statistical analysis may include calculating the mean and variance of the blood glucose levels measured during the first and second therapy periods, respectively. In some examples, an improvement may be determined by comparing the mean value and the variance of the blood glucose levels measured during the first and second therapy periods. In some examples, an improvement may be determined by comparing the mean value and the variance of the blood glucose levels measured during the first and second therapy periods with one or more reference values. The reference values may be values provided by a health care provider or a user and may be stored in the memory 1130 of the glucose level control system 1102. In some examples, the first and second therapy period may be long enough to include a plurality of therapy deliveries (e.g., infusion of glucose and/or glucagon) during each period. In some embodiments, an improvement may be determined by comparing by other statistical quantities calculated at least in part based on the blood glucose levels measured during the first and second therapy periods. In some such embodiments the statistical quantities may be specific statistical quantities defined for comparing the effects of a therapy (e.g., medicament delivery for controlling the blood glucose level in a subject).

In some cases, the first and/or second may be output to user (e.g., the subject or a parent) via a user interface of the glucose level control system and/or a computing system (e.g., a smartphone, laptop, personal computer, or the like). In some examples, the user may use the determined effect to adjust the value of a control parameter.

In some cases, the value that better manages the blood glucose of the subject 1104 may be output to a user (e.g., the subject or a parent). The user may then configure the glucose level control system 1102 based on the selected control parameter value. Alternatively, or in addition, the glucose level control system 1102 may automatically modify the value of the control parameter. In some cases, the user may be provided with an opportunity to confirm the modification. In other cases, the modification may occur automatically without confirmation. However, the modification may be presented to the user (e.g., the subject or a healthcare provider) and/or logged in a therapy log.

In some cases, the comparison is performed by another computing system that is in communication with the glucose level control system 1102. For example, the glucose level control system 1102 may transmit the glucose level signal, data determined from the glucose level signal, and/or the assessment received from the subject, indicative of the effect of the glucose control, to another computing system, such as a local computing system, a smartphone, or a cloud-based computing system. Further, the glucose level control system 1102 may transmit data associated with the control parameters values and the administering of medicament to the subject 1104 to the computing system. The computing system may determine the value of the control parameter that better manages the blood glucose level subject 1104. The computing system may configure the glucose level control system 1102 with the selected value. Alternatively, or in addition, the selected value may be output to a user who can configure the glucose level control system 1102 with the selected value.

At block 1618, the glucose level control system 1102 provides therapy to the subject 1104 based on the selected value for the control parameter that is selected at the block 1616. The therapy provided at the block 1618 may be provided during a third therapy period that is at some point after the first and second therapy periods. Thus, during the first two time periods, the first and second values may be used, respectively, for the control parameter to determine the value that results in the better outcome or improved care for the subject 1104. During subsequent time periods, the value that resulted in the better outcome for the subject 1104 may be used to provide future care for the subject 1104. Alternatively, a new value that is neither the first or second value may be used to provide subsequent care in an attempt to find a value for the control parameter that may provide a better or improved level of care (e.g., closer to a desired target glucose level for a longer period of time) for the subject 1104.

In some examples, providing therapy to the subject, may include generating a dose control signal to a delivery device(s) 1106 (e.g., infusion pump coupled by catheter to a subcutaneous space of the subject 1104) that delivers an amount of a medicament (e.g., insulin or a counter-regulatory agent) to the subject wherein the amount may be determined by the dose signal.

Providing therapy to the subject 1104 based on the selected value may include configuring the glucose level control system 1102 to provide therapy to the subject 1104 during a third therapy period based at least in part on the active control parameter value. In some cases, configuring the glucose level control system 1102 to provide therapy to the subject 1104 based at least in part on the active control parameter value may end the process 1600. In other cases, the automated glucose control refinement process 1600 may be repeated. Repeating the automated glucose control refinement process 1600 may include using the selected value (e.g., the first or second value from a prior iteration of the automated glucose control refinement process 1600) as the first value when performing the operations associated with the block 1604. The second value generated at the block 1608 may be a new value not used during the prior iteration of the process 1600.

The process 1600 may be repeated until a difference between the first effect and the second effect is less than a threshold difference. Alternatively, or in addition, the process 1600 may be repeated a particular number of iterations, periodically, in response to a command, or in response to determining that the blood glucose of the subject 1104 does not satisfy a particular threshold for a particular amount of time.

In some examples, the process 1600 may be used to modify more than one control parameters of a glucose system (or a control algorithm used by the control system). In some such examples, the process 1600 may be used to adjust a first control parameter during a first modification period starting from block 1602 and ending at block 1618, and to adjust a second control parameter during a second modification period again starting from block 1602 and ending at block 1618. The second modification period may be immediately after the first modification period or delayed by a particular time. In some example, the control system may determine when a second control parameter should be modified following the modification of a first parameter. In some examples, the delay may be determined at least in part based on the measured glycemic control based on the glucose signal (e.g., received from a CGM sensor). In some other examples, the delay may be determined based on input received from a user. In some examples, the modification of the second control parameter may be at least partially determined based on the determined modification of the first control parameter.

In some examples, a third control parameter may be adjusted during a third time period after adjusting the first and the second control parameters. The adjustment of the third control parameter may immediately follow the adjustment of the second control parameter or may occur after a delay. The delay may be determined at least in part based on the glycemic control of the subject after the second control parameter is adjusted. In some examples, the glucose level control system may be configured to sequentially adjust the first and second, or the first, second and third control parameters when the glycemic control of the subject satisfies one or more threshold conditions. In some examples, the duration of the time period during which a control parameter is adjusted may defer from that of the other parameters.

In some embodiments, a modified version of the process 1600 may be used to determine a value (e.g., an optimal value) of a control parameter. In some such examples, after determining the second effect at block 1614, the control system may skip block 1616 and block 1618, and instead obtain a third value for the control parameter. In some examples, this third value may be determined at least in part based on the determined second effect at block 1614. In some examples, this third value may be autonomously determined. Further, in some cases, the third value may be automatically determined. In some cases, the third value is determined based at least in part on a user triggering the glucose control refinement process 1600. In some such cases the control system may determine the third value and present it to the user via a user interface 1124 of the glucose level control system 1102. In some examples, the third value may be provided by a user via a user interface 1124 of the glucose level control system 1102. In some examples, after obtaining the third value, the system may provide therapy to the subject based on the third value. This modified version of process 1600 may be repeated several times. In some examples, this modified version may be repeated until a difference between the last two subsequent effects is less than a threshold difference. Alternatively, or in addition, the modified version of the process 1600 may be repeated a particular number of iterations, periodically, in response to a command, or in response to determining that the blood glucose of the subject 1104 does not satisfy a particular threshold for a particular amount of time.

As described, the process 1600 may be used to modify one or more control parameters that affect the delivery of insulin. However, the process 1600 is not limited as such and may be used to modify one or more control parameters that affect the delivery of other medicaments, such as counter-regulatory agent (e.g., glucagon, dextrose, etc.). In some cases, the process 1600 may be used to recommend a change in insulin and/or counter-regulatory agent delivery without modifying the delivery. This can be advantageous for generating recommendations regarding counter-regulatory agent in a single hormone glucose level control system 1102 that does not support counter-regulatory agent, or that supports the use of counter-regulatory agent, but does not have the counter-regulatory agent available.

Moreover, in cases where the process 1600 is used to modify multiple control parameters, the at least two or more of the control parameters may be related to each other. For example, if the control parameters include the time constants α1 and α2, there may be a relationship between α₁ and α₂ such that modifying α1 may cause a modification to α2. For instance, α₂ may equal 1.5 times α₁.

The value for the control parameter set as the active parameter (e.g., the first value or the second value) at the block 1616 may be used by the control algorithm to provide therapy to the subject 1104 for a particular period of time or until the process 1600 is repeated. As previously explained, in some cases, the process 1600 is repeated periodically and/or in response to a trigger, such as a blood glucose value or an average blood glucose value over a time period, or an indicate of a site change for the connection of the glucose level control system 1102 to the subject 1104 (e.g., a change in the location of the infusion set used to provide the subcutaneous dose).

### Hypothetical Example

As previously described, the peak time of absorption of insulin may be referred to as Tmax. Different types of insulin may result in different amounts of time until peak absorption into the subject's blood or for different subjects. For example, in one hypothetical example, the aggregate Tmax among subjects for the fast-acting insulin lispro and insulin aspart may be determined to be approximately 65 minutes, while the aggregate Tmax among subjects using ultra-fast-acting insulin, such as, for example, the insulin aspart injection marketed under the Fiasp brand, which has a formulation to decrease time to peak absorption, may be determined to be approximately 40 minutes. When using an automated blood glucose level control system (such as the glucose level control system 1102) with a control parameter corresponding to Tmax set to 65 minutes, there may be no statistically significant improvement in the average glucose level or the frequency of hypoglycemia when using the ultra-fast-acting insulin compared to using the fast-acting insulin. In this comparison, Tmax is held constant while varying the type of insulin used.

When adjusting the value of the control parameter for the automated blood glucose level control system to use different Tmax settings, in a hypothetical example, mean glucose drops when Tmax is lowered when using the ultra-fast acting insulin. In this example, three cohorts of subjects employ control algorithms that use modified Tmax values when using a glucose level control system with ultra-fast-acting insulin such as Fiasp. The first cohort uses a blood glucose level control system configured with a Tmax of 65 minutes for a first week of therapy and a lower Tmax (such as, for example, 50 minutes) for a subsequent week of therapy. The second cohort uses the blood glucose level control system configured with a Tmax of 65 minutes for the first week of therapy and an even lower Tmax (such as, for example, 40 minutes) for a subsequent week of therapy. The third cohort uses the blood glucose level control system configured with a Tmax of 65 minutes for the first week of therapy and a sharply lower Tmax (such as, for example, 30 minutes) for a subsequent week of therapy. Comparison of the change in Tmax within each cohort and across cohorts demonstrates that the mean glucose level drops when Tmax is lowered, and there is no statistically significant increase or decrease in hypoglycemia.

When Tmax is shorter than physiological insulin absorption peak time, there is an increased risk of hypoglycemia because the blood glucose level control system may stack or administer multiple doses of insulin within a time period. This may occur because the blood glucose level control system may incorrectly identify a lower blood glucose concentration as a maximum blood glucose level concentration when Tmax is set below the actual peak insulin absorption time.

By using the process 1600 to compare the effect of different Tmax settings, it is possible to optimize the Tmax setting for a subject and/or a particular type of insulin. In some examples the comparison may be based on one or more statistical methods. For example, using the glucose concentration data collected during a therapy period (e.g., using a CGM sensor), the control system may determine whether there is a statistically significant difference in mean glucose level during a later period using a different Tmax value compared to an earlier evaluation period. If the subsequent or newer value used for Tmax results in an improved effect, Tmax or a control parameter of the blood glucose level control system 1102 corresponding to Tmax may be set to the newer value, where the change in the control parameter value may occur automatically upon determination of a statistically significant improvement or may occur after generating a notification of the potential improvement and receiving confirmation that the change in control parameter value should occur. After collecting glucose signals of the subject 1104 for a period of time at a default or prior value for Tmax, the value for Tmax may be lowered by a significant amount from the initial Tmax. For example, the control algorithm may automatically change Tmax or an associated time constant to reflect a Tmax reduction of at least 10 minutes, at least 5 minutes, at least 2 minutes, no more than 15 minutes, no more than 20 minutes, no more than 30 minutes, or by a change within a range spanning between any two of the preceding values in this sentence, where the preceding values are included in the range. The system can perform a statistical analysis between the prior data set associated with the higher Tmax, and the current data set associated with the lower Tmax. If the controller of the blood glucose level control system determines that there is a significant or statistically significant improvement (e.g., more than a threshold improvement) in the mean glucose level for the subject with little or no increase in hypoglycemia events or risk events, the system can adopt or recommend the lower Tmax value as the preferred Tmax. This process can be repeated using additional reductions in Tmax. In some cases, each reduction in Tmax may be smaller than the previous reduction. Moreover, if it is determined that there is a not an improvement in the mean glucose level for the subject and/or if there is an increase in hypoglycemia or hypoglycemia risk events, the system may use the prior Tmax or may select a Tmax between the new Tmax and the prior Tmax. Thus, using the process 1600, the system can iteratively modify Tmax to find an optimal value for the subject and/or the selected insulin type.

Moreover, by performing real-time analysis and optimization of one or more control parameters, maintenance of the subject's diabetes can be improved faster and more accurately compared to delayed analysis that may occur during clinical testing. Clinical testing may be less accurate as physiological changes in the subject may not be captured in real time.

In some cases, the real-time process and statistical analysis described above can be used to analyze other types of biomedical data obtained by one or more subject sensors (e.g., measuring one or more physiological parameters). In some such cases, the additional biomedical data, such as data may be received from a smartwatch (e.g., blood pressure, heart rate), from a weight sensor, or any other type of biomedical sensor. By adapting the process 1600 to perform statistical analysis of the additional biomedical data, it is possible to perform a quantitatively objective analysis of biometric data, which can be used by a healthcare provider to care for a subject.

Further, the outcomes of the comparative analysis described above may be used to make additional recommendations to the subject. For example, if it is determined that the actual Tmax for a particular type of insulin is higher than expected for the subject, it may be recommended that the subject modify his or her diet in a particular manner while using that particular type of insulin.

### Example Simulations

Embodiments of an automated glucose level control system 1102 that can be adapted for use with embodiments of the present disclosure are described in International Publication No. WO 2015/116524, published on Aug. 6, 2015; U.S. Pat. No. 9,833,570, issued on Dec. 5, 2017; and U.S. Pat. No. 7,806,854, issued on Oct. 5, 2010, the disclosures of each of which are hereby incorporated by reference in their entirety for all purposes.

The automated glucose level control system 1102 can autonomously administer insulin doses and account for online accumulation of insulin doses (“insulin on board”) due to the finite rate of utilization of the insulin. The rate the insulin absorption, and in turn accumulation, of insulin doses may be modeled by a pharmacokinetic (PK) model (e.g., the bi-exponential PK model represented by equation 2 with preset values of time constants α1 and α2). Of significant clinical significance in relation to the PK model is the time it takes for an insulin dose (e.g., administered subcutaneously) to be absorbed in subject's blood. In some examples, the peak time for insulin absorption in blood is referred to as Tmax. In some other examples. In some other examples, Tmax may be the time at which the concentration of insulin reaches its maximum value following the delivery of a specific dose of insulin. In some such examples, Tmax may be measured from the time that insulin is provided to the subject (e.g., subcutaneously using an infusion set).

In some examples, setting the time constants in the PK model (e.g., α₁ and α₂ in equation 2) may be equivalent to setting Tmax that is inherently assumed by the model; conversely, setting Tmax may set the time constants of the PK model. Since the values of the time constants may be used to determine the online calculation of the accumulation of insulin by a control system, the value of the time constants may consequently control the control system's insulin dosing response to a given blood glucose level excursion. Thus, varying Tmax or time constants associated with Tmax controls the aggressiveness of the control system's insulin doses.

In certain embodiments, the control system implements a method to adapt the control system's PK model's Tmax (hence time constants) setting online. This method may be performed either by the control system periodically making online assessments and calculations that produce recommendations of modifications in Tmax or by the control system autonomously modulating Tmax online. In either case, the calculations may be based on the control system's performance over some time period. In some cases, adaptations to Tmax online, whether autonomously occurring or issued as recommendations can be based on the glucose-control performance by the control system over some time interval, including trends in glucose level, mean glucose level, or extent and/or duration of low glucose level (hypoglycemia) and/or high glucose level (hyperglycemia) occurrence. Alternatively, the calculation can be based on the usage of a counter-regulatory agent, the otherwise intended usage of a counter-regulatory agent had it been available (e.g., in insulin-only systems or in cases where the counter-regulatory agent or its delivery channel are temporarily unavailable). The method can impose upper and/or lower (static or dynamic) bounds for the range over which the Tmax can vary. The degree of adaptation in Tmax for a given situation can be different depending, for example, on the specific insulin being administered by the control system.

In certain embodiments, the described method may be applicable regardless of whether the continuous glucose monitor (which can provide the input glucose signal to the control system) is online or offline. For example, the method disclosed herein can be applied to system described in International Publication No. WO 2015/116524. Further, the described method can coexist with other aspects of the system being activated or not, such as, but not limited to, having a glucose target that is adapted automatically by the system, e.g., as in the system described in International Publication No. WO 2017/027459, published on Feb. 16, 2017, which is hereby incorporated by reference herein for all purposes.

As previously described, the absorption of subcutaneously administered insulin into blood may be governed by the bi-exponential PK model of equation 2. Setting the time constants in the PK model may set a measure of the pending effect of the accumulated amount of insulin in the subcutaneously administered dose, as that can be taken to be the difference between the total area (∫₀^{∞}p(t)dt, which can describe a measure of the total action over time due to a dose U0) and ∫₀^{t}p(t)dt, which can represent a measure of the expended portion of U0. The peak time, Tmax, of the absorption of insulin doses into blood may be given by equation 3. Thus, setting Tmax may set the PK model time constants, which can directly govern the magnitude (e.g., aggressive or conservative) of the control system's online insulin dosing response to a given glucose profile. Although not limited as such, for simplicity, assume that α₁ and α₂ are related, e.g. α₂=1.5 α₁.

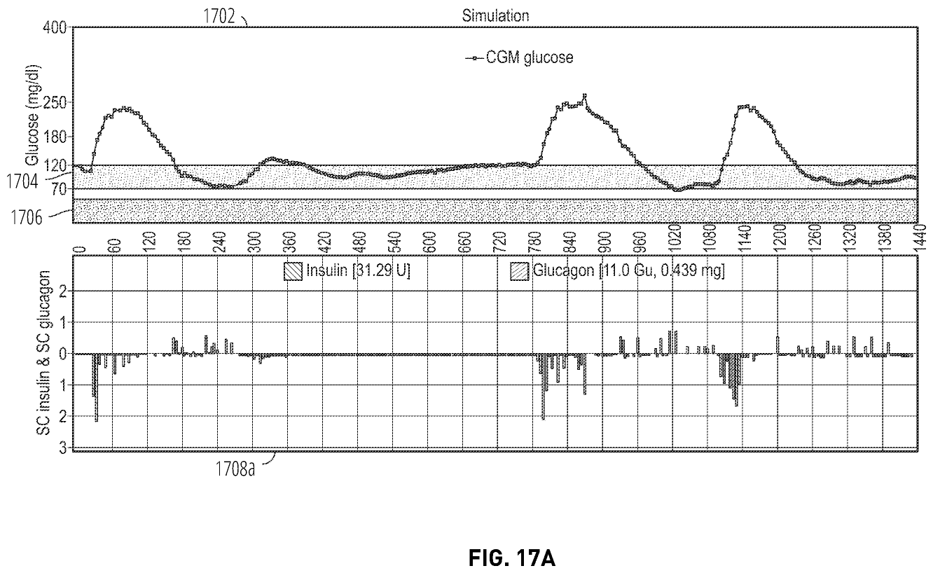

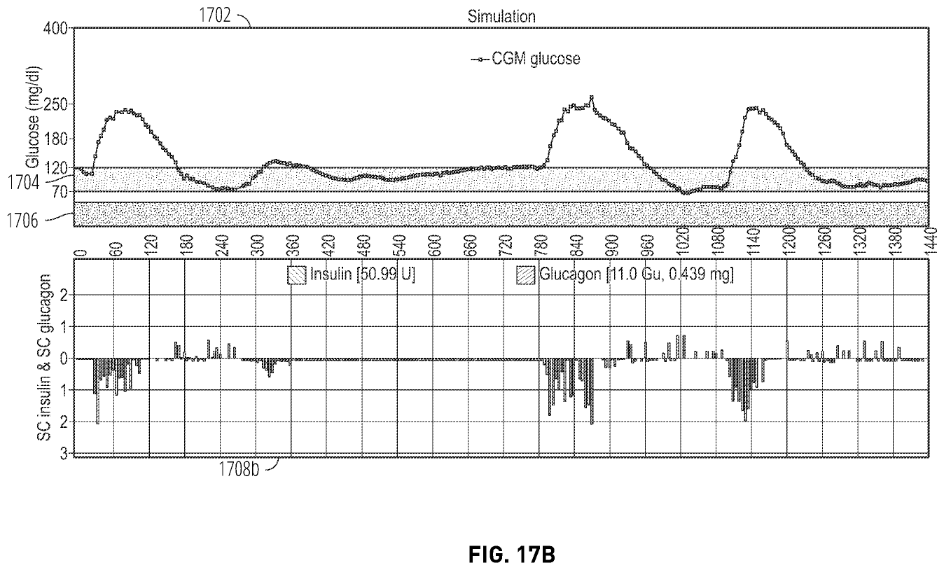

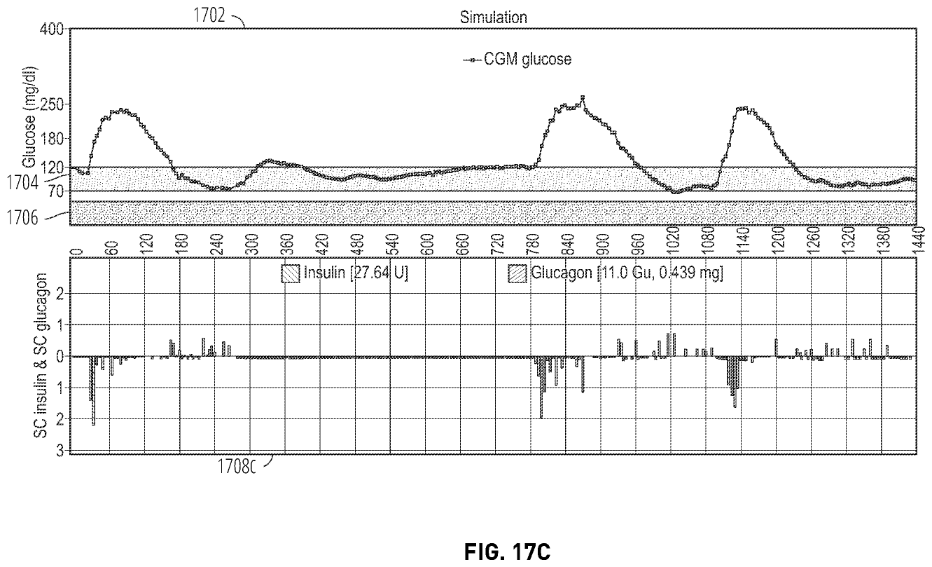

The bi-exponential PK model may be used to simulate the relation between a glucose profile and the medicament (e.g., insulin or glucagon) doses delivered to a subject. FIG. 17A-FIG. 17C illustrate a simulation demonstrating an effect that increasing or decreasing the Tmax setting, or value for a control parameter corresponding to Tmax, may have on the glucose level control system's glucose level control system 1102 online insulin and glucagon dosing response to a given glucose profile (e.g., temporal variation of blood glucose level over 24 hours).

FIG. 17A illustrates a simulation of glucose control of a subject with Tmax set to 65 minutes. The graph 1702 illustrates the variation of blood glucose level (BGL) of a subject over 24 hours. The range 1704 indicates the desired target setpoint range (e.g., between 70 and 120 mg/dL) for the subject's blood glucose level. Further, the range 1706 indicates the range in glucose level (e.g., below 60 mg/dL) for the subject that is associated with hypoglycemia or a risk of hypoglycemia. The graph 1708a illustrates the administering of medicament (insulin or glucagon) to the subject over the same 24-hour time period as graph 1702 based at least in part on the blood glucose level variation illustrated in the graph 1702.

FIG. 17B illustrates a simulation of glucose control of a subject with Tmax set to 15 minutes. The graph 1708b corresponds to the graph 1708a, but with Tmax set to 15 minutes instead of 65 minutes. As illustrated by comparing the graph 1708b to 1708a, reducing Tmax to 15 minutes may result in an increase in insulin dosing required to maintain the given glucose profile 1400.

FIG. 17C illustrates a simulation of glucose control of a subject with Tmax set to 130 minutes. The graph and 1708c corresponds to the graph 1708a, but with Tmax set to 130 minutes instead of 65 minutes. As illustrated by comparing the graph 1708c to 1708a, increasing Tmax to 130 minutes may result in a decrease in insulin dosing required to maintain the given glucose profile 1400.

Even if the glucose profile of a subject is unchanged, increasing or decreasing insulin (or counter-regulatory agent) dosing may affect care of the subject 1104. For example, the subject may experience different degrees of symptoms (e.g., dizziness, nausea, etc.) attributable to maintenance of the subject's diabetes. Advantageously, autonomous optimization of one or more control parameters of a glucose level control system, may reduce the amount and/or frequency of the medicament doses required to maintain a normal glucose profile.

The simulations illustrated in FIG. 17A-FIG. 17C illustrate one non-limiting example of the impact of modifying a control parameter of a glucose level control system. In some cases, different dosing may subsequently lead to different blood glucose excursions which in turn may vary the determined insulin-glucagon doses subsequently. Nonetheless, the simulations shown in FIG. 17A-FIG. 17C, demonstrate the correlation between Tmax as a control parameter and the determined medicament doses by the glucose level control system 1102 for each therapy. Further these simulations demonstrate that the determined therapy doses may be used as a feedback to adjust Tmax as descried below.

Example Automated Glucose control Refinement Process

In some implementations, the value of Tmax can be varied automatically online based on glycemic control in a receding time period. For example, Tmax can be described using the following the equation:

Tₘₐₓ(k)=Tₘₐₓ^{o}+f(yₖ,gₖ), (4)

where Tₘₐₓ^{o} is a baseline value of Tmax, f(yₖ, gₖ) is a parameter control adjustment max function (herein referred to as adjustment function), based on glycemic control of the glucose signal, yₖ, and/or the amount of counter-regulatory dosing, gₖ, that is computed by the control system (whether delivered or not). Evaluation of f(yₖ, gₖ) could be over a time period (e.g., one week, two weeks, four weeks or other time intervals). For example, f(yₖ, gₖ)=Σ_{k-N}^{k}f(yₙ, gₙ). In some examples, k may represent a current therapy period and N may indicate a receding time period that may include one or more therapy periods.

The parameter control adjustment function f(yₖ, gₖ) can cause an increase in Tₘₐₓ(k) relative to Tₘₐₓ^{o} for an increase in hypoglycemia (in severity and/or duration) or impending hypoglycemia in glycemic control of the glucose signal, yₖ, over the receding time period (that may include one or more therapy periods) and, conversely, can cause a decrease in Tₘₐₓ(k) relative to Tₘₐₓ^{o} for an increase in hyperglycemia (in severity and/or duration) in glycemic control of the glucose signal, yₖ, over the receding time period. Moreover, f(yₖ, gₖ) can cause an increase or decrease Tₘₐₓ(k) in relative to Tₘₐₓ^{o} respectively for an increase or decrease in amount of counter-regulatory dosing, gₖ, over the receding time period. The adjustment f(yₖ, gₖ) to Tₘₐₓ(k) can be evaluated and effected at discrete times, which can be at scheduled periodic intervals (e.g., once every 24 hours, once every three days, once a week, etc.), in response to a user command, or based on a physiological measurement of the subject. Alternatively, or in addition, adjustments can be evaluated and effected online when some metric satisfies a threshold or meets certain criteria within the current computation window (e.g., a week, a month, etc.). This criterion can include when hypoglycemia in yₖ reaches or crosses a certain threshold or the level of counter-regulatory dosing in gₖ reaches or crosses a certain threshold. Alternatively, or in addition, the adjustment can be effected after some evaluation related to the glucose signal yₖ (e.g., mean value) in the current computation window has attained a statistically significant difference from its evaluation in a preceding computation window (e.g., the week before). These described implementations allow for having dynamic instances that are mathematically determined online as to when Tₘₐₓ(k) gets adjusted and/or the magnitude by which it is adjusted.

In some examples, therapy periods can be scheduled regular or periodic time intervals (e.g., 24 hour periods, two day periods, one week periods, etc.), based on a user command, or based on a physiological measurement of the subject. In some other examples, therapy periods may be defined as the time interval between two subsequent therapy deliveries, and each therapy period may be identified based on the therapy delivery time that marks the beginning of the therapy period. In either case, f (yₖ, gₖ) may be the adjustment to Tₘₐₓ for the k^{th} therapy period and f(yₖ, gₖ) may be evaluated based on the equation f(yₖ, gₖ)=Σ_{k-N}^{k}f(yₙ, gₙ) wherein yₙ is the glucose signal measured during the nₜₕ therapy period, gₙ is the computed dose of a counter-regulatory hormone for the nₜₕ therapy period and N indicates the receding time period that may include one or more therapy periods. In some examples, N may be the number of the therapy periods receding the kₜₕ therapy period.

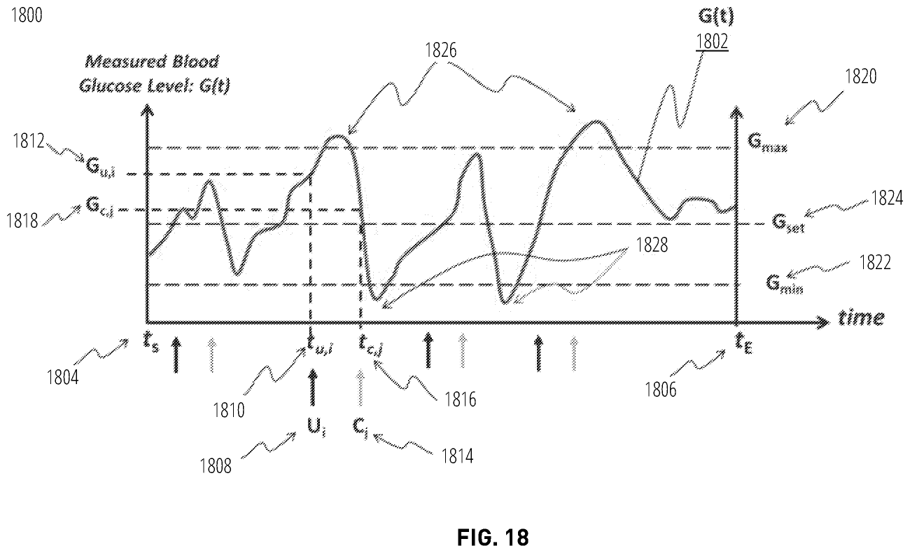

FIG. 18 illustrates a graph 1800 of an example of blood glucose level signal G(t) 1802 (e.g., a CGM trace received from a CGM sensor) over a therapy period (starting from t_{S} 1804 and ending at t_{E} 1806) during which one or several doses of insulin and/or a counter-regulatory agent (e.g., glucagon) are determined and/or administered by the glucose level control system 1102. For example, an insulin dose of Uᵢ 1808 units may be provided at time t_{u,i} 1810 at a measured glucose level of G_{u,i} 1812 (where i varies from 1 to the number of insulin deliveries between t_{S} 1804 and at t_{E} 1806). Similarly, the control system may have calculated a dose of Cⱼ 1814 units, that may have been administered or not, a glucose level G_{c,j} 1818 at which glucagon may have been delivered and the time t_{c,j} 1816, at which glucagon may have been delivered, (where j varies from 1 to the number of glucagon deliveries between t_{S} 1804 and at t_{E} 1806). The control system may be configured to provide therapy in order to maintain the BGL within a normal range defined by an upper bound Gₘₐₓ 1820 and a lower bound Gₘᵢₙ 1822 and close to a Gₛₑₜ 1824. In some examples, the glucose levels above upper bound Gₘₐₓ 1820 may indicate hyperglycemia and glucose levels below lower bound Gₘᵢₙ 1822 may be considered hypoglycemia. For example, during the therapy period shown in FIG. 18, two instances of hyperglycemia 1826 and two instances of hypoglycemia 1828 may be identified by the control system. In some examples, during each therapy period the control system may store blood glucose level signal G(t) 1802, t_{u,i} 1810, t_{c,j} 1816, Uᵢ 1808 and Cⱼ 1814, for all therapy deliveries (all values of i and j). In some examples, the value of one or more control parameters (e.g., Tmax, Gₛₑₜ) may not change during the therapy period between t_{S} 1804 and t_{E} 1806.

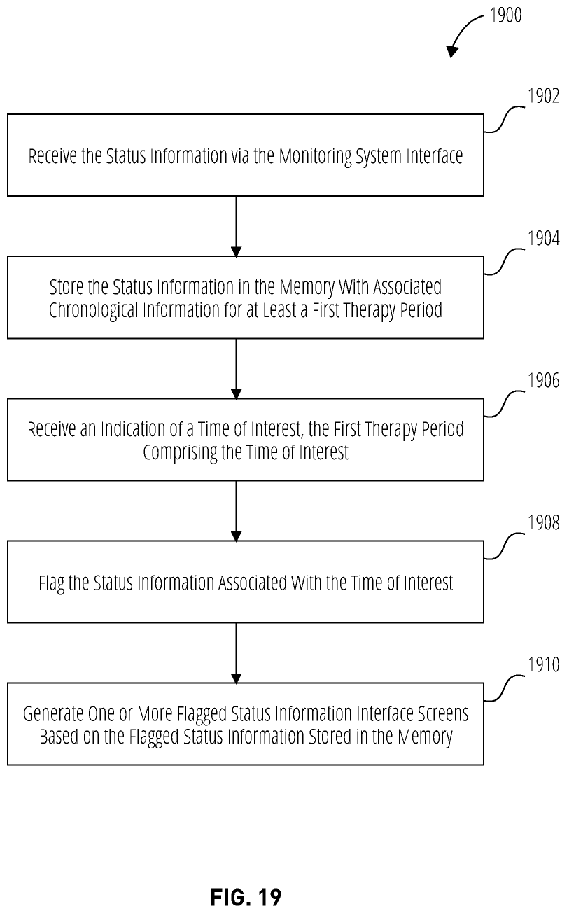

FIG. 19 shows a flow diagram illustrating an example method 1900 that may be used by a control system (e.g., the glucose control system 308, the glucose control system 200b, the AMP 600, the glucose level control system 1102) to receive status information via a monitoring system interface. At block 1902 the monitoring system interface can be configured to receive status information. The monitoring system interface may be part of another interface described herein, such as the user interface 1124 and/or the user interface system 616. The status information can include one or more kinds of information. For example, the status information can include device information pertaining to or at least associated with a condition of an associated ambulatory medicament device (e.g., AMD 100, AMD 202, AMP 600, AMP 702, ambulatory medical device 500, etc.). Additionally or alternatively, the status information can include or subject information pertaining to a condition of a subject. The control status may flag information when at least one of the condition of the ambulatory medicament device or the condition of the subject satisfies a preset flag condition. Preset flag conditions may include when one or more thresholds are exceeded or other triggering event.

At block 1904 the control system may store the status information in the memory with associated chronological information for at least a first therapy period. In some embodiments, the control system can flag the status information with the chronological information. The control system may transmit the flagged status information to a remote server. The transmitted information may be used to identify an issue or problem associated with the use of the ambulatory medicament device and/or associated with the function of the ambulatory medicament device. The chronological information can include a time associated with when the information was obtained. The time associated with the chronological information can include a past time and/or a current time. For example, the chronological information may include real-time information, which may be beyond the first therapy period.

As noted above with regard to FIG. 11, the first therapy period may be based at least in part on a glucose level signal received by the control system and/or on a first value of a control parameter, which may include any control parameter that is used to generate a dose control signal to cause the to administer a dose of insulin or other medicament. The first therapy period may be a time period that corresponds to a single medicament dose or a time period that encompasses a plurality of medicament doses. Additionally or alternatively, the time first therapy period may be a time period associated with a defined length of time, based on a number of medicament delivery periods, and/or based on a specified number of doses of medicament (of any type or of a particular type).

At block 1906 the control system can receive an indication of a time of interest. The first therapy period can include the time of interest. The indication of the time of interest may be received via a user interaction request. The user interaction request may be received via a user interface described herein, such as via the monitoring system interface. The monitoring system interface may be configured to display one or more screens, as described below. The time of interest may be between about 30-90 seconds, between about 1-45 minutes, between about 1-8 hours, between about 1-20 days, and/or between about 1-3 weeks, as specified via user interaction (e.g., with the user interface).

The indication of the time of interest may be determined in response to a triggering event. The triggering event may be one or more events that initiate a sequence of events. For example, the triggering event may be a user interaction with the ambulatory medicament device, a device status change, and/or a change in a subject health status. For example, the triggering event can include at least one of a determination of a hypoglycemia event or other subject health status event.

At block 1908 the control system can flag the status information associated with the time of interest. The control system may flag the status information in response to the receipt of the indication of the time of interest and/or based at least in part on the associated chronological information. The flagged status information may be transmitted remotely, such as to a remote computing system. The transmission may be done via a wide area network transceiver, such as is described above. The wide area network transceiver can be configured to communicate via the wireless wide area network using an address of the remote computing system of a networked computing environment. In some embodiments, the address of the remote computing system can be obtained from a server configured to store the status information. The transceiver may be configured to support communication over one or more communication standards. The one or more communication standards can include one or of a low power wide area network (LPWAN) communication standard, a Narrowband Long-Term Evolution (NB-LTE) standard, a Narrowband Internet-of-Things (NB-IoT) standard, and/or a Long-Term Evolution Machine Type Communication (LTE-MTC) standard. The control system can encrypt the status information and/or transmit the encrypted status information to the remote computing system.

The status information can include therapy data related to therapy delivered by the ambulatory medical device to the subject. The therapy data can include additionally or alternatively at least one of dose data or subject data. The dose data can corresponds to one or more doses of medicament provided by the ambulatory medical device to the subject. The subject data can correspond to one or more medical and/or physiological states of the subject as determined by the ambulatory medical device. The physiological characteristics of the subject may be provided by the readings of a sensor (e.g., the subject sensor 808) and/or according to parameter values received.

The flagged status information may be annunciated to a user, such as displayed via a user interface. For example, at block 1910 the control system may generate one or more flagged status information interface screens (e.g., via the monitoring system interface) based on the flagged status information stored in the memory. The one or more flagged status information interface screens can display the flagged status information and/or the associated chronological information. The display may include a replay of device operation history corresponding to the time of interest. The monitoring system interface may include other screens. For example, the monitoring system interface may generate a replay request interface screen. The control system may receive the indication of the time of interest via user interaction with the replay request interface screen. The control system can selectively transmit the flagged status information and/or non-flagged status information to the remote computing system (e.g., via user interaction). Additionally or alternatively the control system can recognize and store information (e.g., location information, chronological information, therapy data, triggering events, etc.) of the ambulatory medicament device when storing the flagged status information. For example, the control system can synchronize one or more elements of the stored information (e.g., a recording and time plot), such as at least one of the therapy data, one or more triggering events, a glucose level time plot, and/or alarms.

In some embodiments the control system can automatically crop out certain information (e.g., non-flagged status information) and generate the flagged status information without the non-flagged status information and/or generated the flagged status information within the first therapy period. For example, the control system may discard the flagged status information within the time of interest (e.g., via user interaction with the monitoring system interface).

The method 1900 can include determining a triggering event from the flagged status information. The triggering event can include at least one of a device malfunction, a subject abnormality, or a clinical event. In response to determining the triggering event, the control system may be configured to determine whether the determined triggering event requires urgent resolution and/or whether the determined triggering event can be resolved by the user. In response to determining that the determined triggering event does not require urgent solution and can be resolved by the user, the control system can set an alarm at a single or recurring time interval, the alarm being at least one of visual, auditory, and/or haptic annunciation. In response to determining that the triggering event requires urgent resolution, the control system can provide an alarm (e.g., via the ambulatory medicament device) to alert the user and/or transmit an alarm signal to a central server or a customer service. In response to determining that the triggering event is the device malfunction and/or that the determined triggering event requires urgent resolution, the control system may halt operation of the ambulatory medicament pump. This can reduce or prevent potential injury to a subject, such as by failing to perform an expected operation or by performing an operation that was not authorized. In some embodiments the control system can discard the flagged status information (e.g., in response to user interaction) when the triggering event is resolved. This can free up memory that is no longer needed and/or improve performance of the control system by speeding up processing. Conditions for determining whether the triggering event requires urgent resolution can include one or more criteria, such as troubleshooting, pump motor failing, incorrect insulin, and/or in a case in which a control algorithm does not meet a desired level of glycemic control

The control system can provide an instant alarm notification based on a user setting. The instant alarm notification may be at least one of visual, auditory, and/or haptic annunciation. The determined triggering event may include one or more device malfunctions. Device malfunctions can include at least one of: a battery failing to meet a manufacturer's specification, an input and output communication error, an electrical failure, a mechanical failure, a fluid pressure outside a pressure threshold, a pump controller malfunction, an error associated with the non-transitory memory, the pump exceeding a manufacturer's warranty, the pump having an indication of tampering, the pump exceeding a usage threshold criterion, and/or the pump being subject to a manufacturer recall. In response to determining that the triggering event is the device malfunction, the control system may perform a diagnostic test on the ambulatory medicament pump. As noted above, the triggering event may include a subject abnormality, which can can include one or more of: a change in meal consumption schedule, a heart rate outside a threshold (e.g., below a minimum threshold, above a maximum threshold), euglycemia being outside a threshold, and/or a clinical event having a glucose level outside a threshold.

The status information can include a threshold or threshold value described above. The control system can flag the status information associated with the time of interest and/or store the flagged status information for a triggering event time period that includes the triggering event. The control system can display (or otherwise annunciate) the flagged status information during the triggering event time period. The triggering event time period may include a certain amount of time before and/or after the instance of the triggering event. For example, the time may between about 30-90 seconds, between about 1-3 minutes, between about 5-15 minutes, between about 0.5-3 hours, and/or any time therebetween.

In some embodiments, the method 1900 can include outputting speaker signals. For example, the control system can include a speaker controller that is configured to generate annunciations on a speaker of the ambulatory medicament device. The control system may generate one or more flagged status information annunciations from the flagged status information stored in the memory. The one or more flagged status information annunciations can be indicative of the flagged status information with chronological information as the replay of device operation history associated with the time of interest. For example, the speakers may provide a verbal description of the flagged status information, the chronological information, and/or other information. The speaker controller may be configured to annunciate the one or more flagged status information annunciations on the speaker to illustrate via the speaker the replay of device operation history.

In some embodiments, the display controller includes a touchscreen controller (such as the touchscreen controller 1128). The control system may be configured to generate a replay request interface screen using the touchscreen controller and receive the indication of the time of interest via user interaction with the replay request interface screen.

In some embodiments, the control system includes a haptic controller that is configured to output haptic signals that are configured to generate vibrations on the ambulatory medicament device. The control system can generate one or more flagged status information vibrations. The flagged status information vibrations may be based on the flagged status information stored in the memory. The one or more flagged status information vibrations can be indicative of the flagged status information and the associated chronological information as the replay of device operation history associated with the time of interest. The haptic controller can be configured to annunciate the one or more flagged status information vibrations on the haptic controller to illustrate via the haptic controller the replay of device operation history. For example, Morse code or other signaling code may be used to annunciate the replay of the device operation history. The haptic controller may be configured to control the haptic signals to be different based on preset severity levels. For example, the control system can generate vibrations with different intensities based on a severity level of the flagged status information. The severity level can range from 0 to 5, with 0 being least or nonurgent alarm conditions and 5 being urgent or most urgent alarm conditions. The alarm conditions may correspond to alarm conditions described above.

In some embodiments the control system can discard from the memory the stored information not flagged in association with the indication of the time of interest after a predetermined period of time. Additionally or alternatively the control system can store the status information in the memory data with chronological information for at least a second therapy period after the stored information that is not flagged has been discarded, the second therapy period comprising the time of interest. For example, the chronological information associated with the second therapy period may replace the chronological information associated with the first therapy period.

In some embodiments the control system can selectively display one or more flagged status information interface screens. These screens may be displayed in response to user interaction with the monitoring system interface. The one or more flagged status information interface screens can include time data and/or status information comprising the device information and/or the subject information synchronized for the first therapy period. The device information can include cartridge usage information, battery information, and/or the subject information. The subject information may include a glucose level (e.g., in real-time) and/or meal consumption recognition. The one or more flagged status information interface screens may be generated in a simplified graphical representation. The graphical representation can include timeline information. Thresholding may be applied to the status information in the past and/or receding time horizon. The graphical representation includes settings adjustable via user interaction with the monitoring system interface, the settings include screen scale, colors, brightness, and units. download and review the stored status information in the memory for a period of time selected via user interaction with the monitoring system interface. When no triggering event is detected, one or more flagged status information interface screens may be generated on the display in a fast forward manner. Additionally or alternatively the display of any of the flagged status information interface screens may be played forward at a normal speed, played forward in a fast forward speed, paused, and/or played in reverse at any speed.

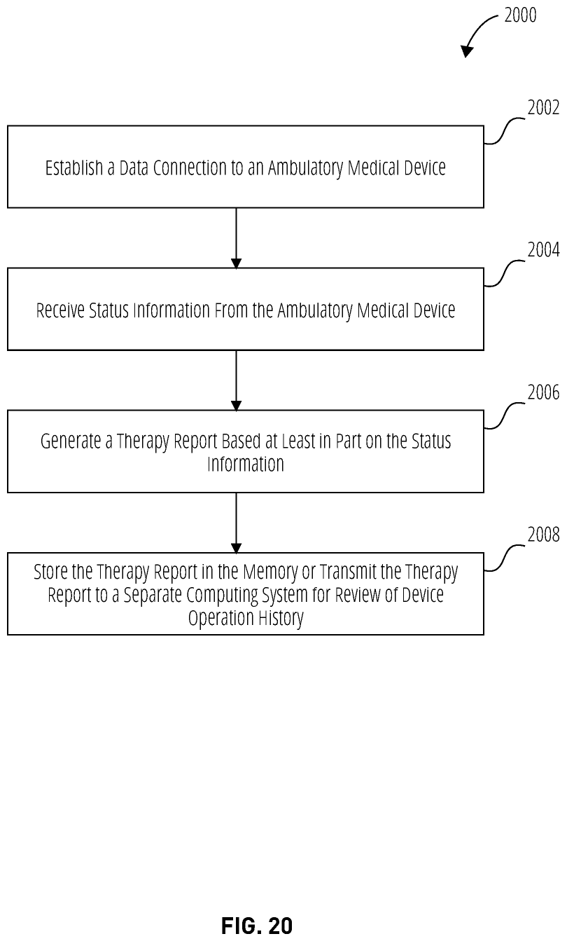

FIG. 20 shows a flow diagram illustrating an example method 2000 that may be used by a control system (e.g., the glucose control system 308, the glucose control system 200b, the AMP 600, the glucose level control system 1102) for reviewing device operation history of an ambulatory medicament device. At block 2002 the control system can receive status information via a monitoring system interface. The monitoring system interface may be part of another interface described herein, such as the user interface 1124 and/or the user interface system 616.

At block 2004 the control system can store the status information in the memory with chronological information for at least a first therapy period. At block 2006 the control system may receive an indication of a time of interest. The first therapy period can include the time of interest. In response to the receipt of the indication of the time of interest, the system can flag the status information associated with the time of interest. At block 2008, the control system can store the flagged status information with chronological information in the memory or transmit the flagged status information with chronological information for review of device operation history.

In some embodiments the control system can generate a therapy report based at least in part on the status information. The therapy report can include time-series therapy data relating to therapy that is delivered by the ambulatory medical device. The time-series therapy data may be obtained and/or generated over a particular time period. The therapy report can additionally or alternatively include a condition of the ambulatory medicament device over a particular time period and/or a condition of the subject for the particular time period. The particular time period may be associated with a time of interest (e.g., described herein). The particular time period may be automatically determined based on the status information. For example, a condition of the subject that exceeds a threshold level described herein (e.g., threshold risk of hypoglycemia) may cause the control system to generate the therapy report starting with or near in time to the threshold level being exceeded. The control system may further store the therapy report in the memory and/or transmit the therapy report to a separate computing system (e.g., remote server) for review of device operation history. The time of interest may be based on an indication for the time of interest received from a user. The control system may display the indication for the particular time period

The control system may receive (e.g., via the data connection from the ambulatory medicament device, via user interaction with a user interface of the control system) an alarm signal indicating a triggering event. The triggering event may include a device malfunction associated with the ambulatory medicament device, a subject abnormality, a clinical event, and/or another triggering or threshold described herein. The alarm signal may include flagged status information obtained by the ambulatory medicament device. The control system can determine (e.g., automatically based on the status information) that a triggering event has occurred, such as any triggering event described herein. For example the triggering event may include a device malfunction associated with the ambulatory medicament device, a subject abnormality, a clinical event, and/or any other triggering event or threshold.

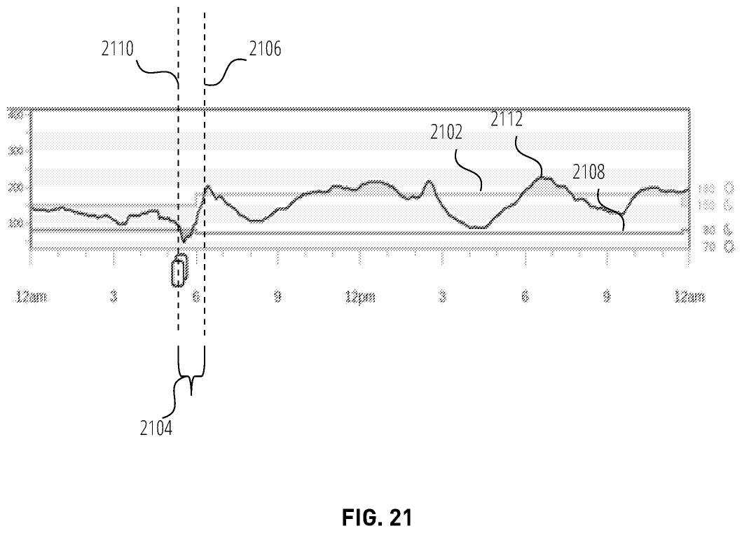

FIG. 21 shows an example graph of a subject's blood glucose level 2112 (in mg/dL) over a 24-hour period. The graph of FIG. 21 may be an example graph that could be displayed by the control system and/or on the ambulatory medicament pump. The graph includes a display of the blood glucose level 2112 that is generally between a minimum threshold 2108 and a maximum threshold 2102. The minimum threshold 2108 and maximum threshold 2102 may change over time based on one or more conditions such as a time of day. For example, as shown in FIG. 21 the minimum threshold 2108 and the maximum threshold 2102 may be based on whether the subject is awake or asleep. Other conditions are possible, depending on the thresholds that apply.

A user may select a time of interest 2104. The time of interest 2104 may be selected via a display interface (e.g., a displaying interface screen). Additionally or alternatively the 2014 may be set automatically, based on one or more of a triggering event 2110 and/or a threshold condition 2106. As shown, the triggering event 2110 corresponds to the blood glucose level 2112 falling below the minimum threshold 2108. The threshold condition 2106 shown in FIG. 21 corresponds to the blood glucose level 2112 rising above the maximum threshold 2102. Other thresholds and/or conditions are possible and these are used for illustration purposes. During the time of interest 2104, the control system may record the results of relevant variables associated with the ambulatory medicament device and/or the subject. The control system may be able to display one or more aspects of the blood glucose level 2112, which may only include just the time of interest 2104 (e.g., in a cropped fashion). Other display options are possible, as described herein.

AMD with Alarm System

In some cases, a condition may occur that impacts the operation of the ambulatory medicament device. This condition may be associated with the ability of the ambulatory medicament device (AMD) to operate as intended by the manufacturer, a subject receiving therapy from the ambulatory medicament device, and/or user (e.g., healthcare provider, parent, or guardian of the subject). In some cases, the AMD may be operating as intended, but the condition of the subject may not satisfy a desired level of health. In either case, it is generally desirable to generate an alarm to inform the subject and/or one or more users of the condition of the AMD and/or the subject. Moreover, it is desirable to track the alarm until the condition that caused the alarm is resolved. Further, it is desirable to issue different types of alarms for different conditions to enable a subject or user to easily distinguish the severity of the condition that triggered the alarm. The user may be a subject receiving medicament or therapy, or may be another user, such as a clinician or healthcare provider, or a parent or guardian.

As mentioned above, an ambulatory medicament device may include an alarm system configured to monitor the ambulatory medicament device and/or the subject, and to generate an alarm when it is determined that a condition has been detected that satisfies an alarm condition. In some examples, the alarm system that may organize a list of alarms, notifying a user of these alarms, and allowing the user to acknowledge alarms.

In some embodiments, the alarm system may comprise a plurality of sensors that monitor the AMD and/or the subject, a monitoring system interface that receives the data from sensors, and alarm annunciation and control system that process the received data and generate alarms if an alarm condition is met. In some examples, the monitoring system interface and the alarm annunciation and control module are implemented using one or more hardware processors and machine readable instructions. In some examples, the monitoring system interface and the alarm generation module are separate hardware modules.

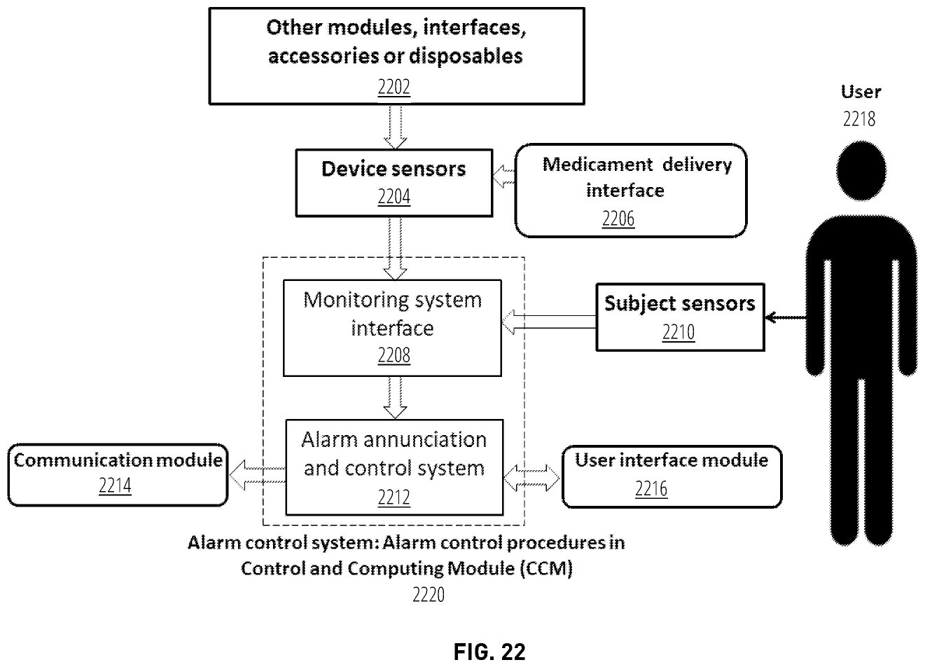

With reference to FIG. 22, in some embodiments, an alarm system 2220 implements alarm control procedures in the controller 602 of the AMD. The 602 may be referred to herein as a control and computing module (CCM). The alarm system 2220 can be implemented as instructions stored in a memory of the CCM (e.g., the memory 610) and executed by a processor (e.g., processor 604) to generate an alarm upon detection of a condition of the ambulatory medicament device and/or the subject. In some cases, the hardware processor of the monitoring system is a hardware processor of the ambulatory medicament device that controls medicament delivery. In some cases, the hardware processor of the monitoring system may be a separate hardware processor.

In some examples, the alarm system 2220 includes a monitoring system interface 2208 and an alarm annunciation and control system 2212. The alarm annunciation and control system 2212 may include sub-systems for determining the severity of an alarm condition, user notification processing and receiving alarm control commands from the user interface module 2216. The user interface module 2216 may include one or more of the embodiments described with respect to the user interface module 1808. The monitoring system interface 2208 may monitor the condition or status of the AMD and/or the subject at least partially based on signals or status values received from a set of device sensors 2204 and a set of subject sensor 2210. In some examples, the device sensors 2204 may be configured to track the status of the components or the elements of the AMD, and the subject sensors 2210 can be configured to obtain measurements of one or more physiological characteristics of the subject.

In some examples, device sensors 2204 are sensors that generate a signal or status value associated with the condition of modules, interfaces, accessories, disposables of the AMD. In some examples, the device sensors 2204 may generate signals that correspond to parameters associated with a component in a module or interface. For example, one device sensor may record the voltage of a battery and another device sensor may record the follow rate of a pump the medicament delivery interface 2206.

In some examples, a subject sensor 2210 may be any sensor that generates a signal or status value associated with one or more physiological indicators (or parameters) of a subject (e.g., heart rate, blood pressure, body temperature, level of blood sugar, serum levels of various hormones or other analytes). In some such examples, the subject sensor can be a continuous glucose monitoring sensor (CGS). The device and subject monitoring system interface 2208 may continuously receive and analyze signals from device sensors 2204 and subject sensors 2210 to determine the condition of the AMD, the subject, a sensor, and/or other accessories.

In some cases, a single sensor may be used to monitor both the condition of the subject and the ambulatory medicament device or accessories and sensors connected to AMD. For example, a continuous glucose monitoring CGM sensor may be used to monitor the condition of the subject, and may also be monitored to determine whether the condition of the CGM satisfies an alarm condition (e.g., to alarm a user that the CGM should be replaced).

Although described as sensors of the AMD, one or more of the sensors may be accessories that may or may not be part of the AMD, but that may communicate with the AMD.

In some examples, the alarm system 2220 implements procedures for allowing a user or the subject to change the alarm settings and/or acknowledging an alarm annunciation via the user interface module 2216. In some examples, the user may be able to see one or more alarms annunciated on a user interface (e.g., as a list of alarms), even if the AMD is in locked state. In these examples, the user may not be able to acknowledge or respond to alarm when the AMD is in locked state.

In some such examples, a user or the subject may get access to an alarm setting screen or acknowledge an alarm annunciation by providing a wake action or a wake action followed by a first gesture on, for example, a touchscreen display. In some cases, the first gesture may be created by entering predetermined or particular characters on the alphanumeric pad. In some such examples, the alarm system 2220 distinguishes inadvertent alarm control inputs from intentional alarm control inputs. An inadvertent alarm control input is an alarm acknowledgment input that was made without the intent of the user 3227 to acknowledge the alarm that the ambulatory medical device 600 is delivering to the user. An example of an inadvertent alarm acknowledgment is one that was accidentally executed by the user 3227 by putting pressure on the ambulatory medical device 600 in the jacket pocket of the user 3227.

In some examples, the alarm system 2220 implements processes for determination and categorization of an alarm condition based on its severity level (e.g., a severity level between 0 and 5), according to the information received through the monitoring system interface 2208. In some examples, once an alarm condition is detected, the alarm annunciation and control system 2212 may place it in the appropriate queue, for example, based on severity or category. In one or more embodiments, a list of alarms may be generated wherein alarms may be sorted numerically in descending order with the highest priority fault displayed at the top.

In some examples, the alarm system 2220 implements procedures for controlling the annunciation of alarm conditions via the user interface module 2216, at least partially, based on their severity level. In some such examples, a user interface (e.g., a touchscreen display) may be configured to allow the user to navigate directly to the issue or fault for which an alarm is being delivered and to address the fault causing the alarm so that it could be corrected thereby stopping the alarm

### Alarm Conditions

In some examples, the device and subject monitoring system interface 2208 may provide a status information received from the device sensors 2204 and/or subject sensors 2210 to the alarm annunciation and control system 2212. In some examples, the status information may comprise one or more status values. In some examples, the status information may comprise device information pertaining to a condition of the ambulatory medicament device or subject information pertaining to a condition of the subject. In some such examples, the alarm annunciation and control system 2212 is configured to determine based at least in part on the status information received from the monitoring system interface 2208, whether an alarm condition is satisfied.

Determining whether the alarm condition is satisfied may include comparing one or more status values associated with the ambulatory medicament device and/or the subject to one or more alarm thresholds or alarm conditions. In some cases, each alarm threshold or alarm condition may be associated with an alarm profile. In some such cases, determining whether the alarm condition is satisfied may include comparing the status information to one or more alarm thresholds or alarm conditions included in one or more alarm profiles. In some examples, the alarm profile may be stored in the storage 618 of the CCM 610. In some such examples, at least some of the alarm profiles may be provided to the CCM by an authorized user or the subject via a user interface or directly transferred from another device to the storage (e.g., from USB drive, a laptop, smart phone, PC and the like). In some examples, at least some of the alarm profiles may be stored in the storage 618 at the time of manufacture,

Each of the alarm profiles may indicate the characteristics or status of the AMD and/or subject that triggers an alarm corresponding to the alarm profile. For example, at least some alarm profiles may indicate the threshold status values below or above which an alarm should be triggered. For example, one alarm profile may indicate that when a blood glucose level of the subject exceeds a particular threshold, a particular alarm is to be generated and/or annunciated. As another example, an alarm profile may indicate that when an available amount of medicament is below a particular threshold, a particular alarm is to be generated and/or annunciated. The type of alarm and/or the alarm frequency or intensity associated with the medicament level may differ from the alarm triggered based on the blood glucose level. Although the previous examples described a single condition associated with a single alarm profile, it should be understood that multiple conditions may be associated with an alarm profile. For example, a blood glucose level that exceeds an upper threshold or is below a lower threshold may be associated with different alarm profiles or the same alarm profile. As another example, a blood glucose level that is above an upper threshold or a medicament pump that is unable to supply insulin may be associated with the same alarm profile. On the other hand, a medicament pump that is unable to supply insulin due to an empty insulin cartridge may be associated with a different alarm profile than if the medicament pump is unable to supply insulin due to damage to the medicament pump.

Some non-limiting examples of conditions of the AMD or of the subject that may be associated with an alarm profile include conditions relating to a battery capacity (e.g., below a threshold charge capacity or below a capacity associated with a particular amount of operating time (e.g., one day)), a battery condition (e.g., high temperature or low voltage), a medicament or drug delivery condition (e.g., medicament is empty or below a threshold, motor is stalled, catheter is occluded, etc.), subject sensor condition (e.g., blood glucose sensor is expiring, or signal was not received from sensor), calibration failure, high or low glucose levels, network (e.g., Bluetooth® or BN-LTE) communication errors, haptic interface errors (e.g., motor non-responsive), speaker errors (e.g., noise or low volume), medicament cartridge errors (e.g., empty cartridge, cartridge detection error, etc.), and the like. As explained below, each of these errors or conditions may be associated with different severity levels that cause the annunciation of different alarms.

In some cases, each alarm profile may be associated with a severity level of the alarm. The severity level may be associated with how urgently the condition that triggered the alarm should be addressed or resolved. Further, the severity level may be associated with an amount of harm that may be caused to a subject if the condition that triggered the alarm is not resolved or is not resolved within a particular time period. The number of severity levels may vary based on the type of ambulatory medicament device. Generally, there is no limit to the number of severity levels. However, there may be a point of diminishing returns as the number of severity levels exceeds a particular number because, for example, it may be difficult for a user to distinguish between the different numbers of severity levels or to identify with which severity level a particular alarm is associated. Thus, the number of severity levels may be limited to a particular number, such as 3, 5, 6, 9, or some number in between. However, it is possible for there to be more than 9 severity levels.

There may be multiple alarm profiles associated with a severity level. Or each condition of the AMD and/or subject that is associated with the same severity level may be associated with the same alarm profile.

The AMD may determine a severity of an alarm condition based on the condition of the ambulatory medicament device and/or the subject that triggered the alarm condition. In some cases, the ambulatory medicament device may determine the severity of the alarm condition based at least in part on an alarm profile associated with the alarm condition.

Generally, if the alarm condition does not prevent the AMD from providing therapy, the AMD may continue to provide therapy. However, in some examples, if the alarm condition interferes with the delivery of therapy, operation of the AMD may be suspended or partially suspended. Generally, alarm conditions that interfere with the provisioning of therapy may be associated with a higher severity level. However, some alarm conditions that interfere with the provisioning of therapy may be associated with lower severity levels. For example, a determination that the AMD cannot supply insulin may normally be associated with a highest severity alarm. But if a user indicates that the site location is currently in process of being changed, the alarm condition may be associated with a lower severity level (e.g., an informational alarm reminding the user that insulin cannot be delivered during site change). In some examples, in response to determining that the severity level of the alarm condition matches an unsafe operation (e.g., a condition that may cause the AMD to provide doses of the medicament that are above or below certain values, or to unreliably determine the subject's condition), the AMD may suspend delivery of the medicament to the subject. Once the condition is resolved, the AMD may resume delivery of medicament to the subject. If on the other hand it is determined that the alarm condition matches a safe operation severity level, the AMD may be configured to maintain delivery of medicament to the subject

### Alarm Annunciation

When an alarm condition is satisfied, the alarm annunciation and control system 2212 can implement an annunciation pattern selected based at least in part on the status information generated by and/or received from the monitoring system interface 2208. The annunciation pattern may be selected from a plurality of annunciation patterns based at least in part on the alarm condition and/or the status information. The annunciation pattern may include one or more different text patterns or text information, audible alarms, visual alarms, or haptic alarms. Determining whether the alarm condition is satisfied may include comparing one or more status values associated with the ambulatory medicament device and/or the subject to one or more alarm thresholds or alarm conditions associated with an alarm profile.

Upon verifying that an alarm condition associated with an alarm profile or alarm condition is satisfied, the alarm annunciation and control system 2212 annunciates the alarm condition. In some cases, at least some of the alarm conditions may be associated with a unique annunciation pattern. Advantageously, by having unique annunciation patterns for at least certain alarm conditions, a user can instantly know the stated of the AMD and/or subject based on the annunciation pattern for the alarm.

In some cases, the AMD may have a wireless electronic communications interface that can be used to transmit an alarm signal, status information, alarm condition data, and/or an annunciation pattern to a remote electronic device. In some such cases, the remote electronic device may annunciate an alarm if an alarm condition is met. The remote electronic device may include any device that can receive alarm information or status information from the AMD. For example, the remote electronic device may be a smartphone, smartwatch, smart glasses, a laptop, a tablet, or any other computing device.

In some examples, the alarm system may generate a list of pending alarm conditions and store it in a memory of the AMD (e.g., storage 618 in CCM 610). In these examples, any time an alarm condition associated with an alarm profile is satisfied, the alarm system may update the list of pending alarm condition by adding the new alarm condition to the list of pending alarm conditions. In some examples, the list of pending alarm conditions may comprise a list of elements (e.g., icons, text, and the like) each indicating an alarm condition (e.g., an alarm condition that has been annunciated). In some examples, the AMD may display an alarm state icon comprising a visual indication of a count of alarm conditions on the list of pending alarm conditions.

In some examples, the list of pending alarm conditions may be sorted according to the severity level associated with the alarm conditions.

In some examples, the alarm system may annunciate the alarm conditions via the user interface module 2216 of the AMD 600. For example, the alarm condition may be annunciated via one or more user interfaces (e.g., a display, a touchscreen display, a speaker, and the like). In some such examples, an alarm may comprise an audio alarm, a text message, a graphical message, a text or graphical message with audio, vibrations, flashing light and any combination of these.

In some examples, the alarm conditions may be transmitted to other devices, via the communication module 2214 of the AMD where, for example, an authorized user (e.g., guardians or parents of the subject), the subject or an emergency provider can view the alarm condition. In some examples, the alarm annunciation and control system 2212, may establish a direct end-to-end connection with a computing system (e.g., a cloud computing system) using the communication module 2214 and send the alarm condition to the computing system through the end-to-end connection.

Based on the severity of the alarm condition and/or the alarm profile corresponding to the alarm condition, an alarm may be generated and/or annunciated that is associated with the severity of the alarm condition and/or the type of alarm condition. Different alarm conditions and/or alarm profiles may result in different types of alarms or different annunciations of the alarm. For example, an alarm associated with the highest severity level may include an audible alarm with a loudness that exceeds a particular decibel level (e.g., above 70 or 80 decibels), a visible alarm (e.g., a flashing or steady light) with a luminance above a particular luminance value (e.g., a luminance between 10⁵ or 10⁶ candelas per square meter), and/or a vibrational alarm. Further, the alarm associated with the highest severity level may not be snoozed or dismissed. Alternatively, the alarm associated with the highest severity level may be snoozed for a shorter time period than alarms of lower severity levels (e.g., for 5 minutes, for 10 minutes, etc.). An alarm associated with a different severity level than the highest severity level may include a different combination of audible, visible, and vibrational alarms. Not only may the existence of audible, visible, and vibrational alarms differ for different severity levels, but so may the characteristics of each of the alarm types. For example, audible alarms may have different sound patterns, loudness, frequencies, etc. Visible alarm may be of different intensity, color, pattern, etc. Vibrational alarms may be of different pattern, intensity, etc. Further, an alarm with a different severity level than the highest severity level may be permitted to be snoozed or dismisses or snoozed for a longer period of time. In some examples, the severity of the alarm condition may determine the type of type of the alarm generated (e.g., audio, text, graphical, or any combination of these).

Further, the display of alarm conditions on the user interface may include an icon for each type of alarm condition. The user interface may display the number of alarm conditions and/or the number of alarm conditions of a particular type or severity level. In some cases, a duplicate alarm may be omitted from the list of alarms. In some cases, a count of the occurrence of alarms may be increased to reflect the duplicate alarm. In some cases, a duplicate alarm may result in the annunciation of the duplicate alarm. In some cases, the duplicate alarm is ignored. In some cases, the occurrence of a duplicate alarm may cause an escalation of the existing alarm. For example, if an alarm condition that causes an annunciation of an alarm with a first severity level is detected as occurring a second time, the alarm may be annunciated with a second severity level that indicates a greater degree of severity than the first severity level. It should be understood that an alarm occurring after an alarm condition is resolved may not be considered a duplicate alarm, but instead may be a reoccurrence of the alarm condition and/or an indicator that the resolution for the alarm condition failed (e.g., an insulin cartridge replacement is faulty or is empty)

In some cases, the list of alarms may be observed via a user interface (e.g., a touchscreen display) when the user interface is locked. In some such cases, further, details about the alarms may be accessible when the user interface is locked. In some cases, in order to access more details about the alarms and/or resolve the alarms, it may be necessary to unlock the user interface unlocked (e.g., by a wake action and/or a gesture).

Each of the alarm conditions, or information associated therewith, may be added to an indicator or user interface (e.g., a list, or other data structure or user interface element) that may be accessed by a user. This user interface may maintain the alarm condition on the user interface until the alarm condition is resolved. Further, the alarm conditions may be sorted or ranked based on the severity level of the alarm condition, the time that the alarm condition occurred, whether the alarm condition relates to the subject or the ambulatory medicament device, any combination of the foregoing, or any other factor for sorting or ranking the alarm conditions.

In some cases wherein the alarm is presented on a display, the displayed information may include details about what caused the alarm, the severity of the alarm, how to respond to or address the alarm, or any other information that may be informative regarding why the alarm was generated and/or how to respond to the alarm. In some cases, the information may provide a workflow or instructions on how to respond to the alarm. The instructions may include a link to a workflow provided by a manufacturer of the ambulatory medicament device or of another entity, such as an entity that provides medicament or site changing kits.

In some cases, different views of an alarm or different information associated with the alarm may be provided based on an identity of the user, or a role of the user, viewing the alarm. For example, a child may be instructed to contact a parent to address an alarm. But a parent may be provided with information to resolve the alarm. The parent may receive simplified information (e.g., blood glucose level is high) about what caused the alarm, but a healthcare provider may receive more detailed information regarding the alarm (e.g., internal control parameter values, insulin flow rates, curvature of insulin diminishment predictions, etc.) that facilitates the healthcare provider caring for the subject.

The alarm conditions may be displayed on a display of the AMD. Alternatively, or in addition, the alarm conditions may be displayed on a remote display that is separate from the ambulatory medicament device. The remote display may be a display that is authenticated or associated with a computing device that is authenticated to access data, such as alarm conditions, from the AMD. In some cases, the list of alarms may be presented on a mobile device (e.g., a smartwatch or smartphone) or on a computing device (e.g., a laptop or desktop) that can obtain data directly or indirectly from the AMD.

In some cases, annunciating the alarm may include contacting a manufacturer and/or user (e.g., a healthcare worker, a parent or guardian, or other registered user). Further, the alarm may include instructions on repairing the ambulatory medicament device and/or on addressing the alarm condition. For example, the alarm may provide a user with instructions to replace the insulin cartridge and how to replace the insulin cartridge. As another example, the alarm may provide instructions on how to change the battery of the device or on how to change a site where the insulin pump connects to the subject. In some cases, the alarm may include one or more operations associated with the alarm. For example, the alarm may trigger reordering of insulin or may request that the user confirm a reorder request to reorder insulin.

### Resolving an Alarm

Certain alarms, such as informational alarms, may be dismissible. However, generally the alarm may remain on the alarm list until the condition that caused the alarm is resolved.

A user may be able to acknowledge and/or snooze alarms via a user interface. In some examples, in order to acknowledge and/or snooze alarms, the user may first need to activate the user interface (e.g., by providing a wake action) and then provide a gesture to unlock the user interface. For example, the user may use the wake button to activate a touchscreen display and then provide a gesture on the screen to unlock display. In some example, the touchscreen display may be configured to allow the user or subject to navigate directly to the issue or fault for which an alarm is being delivered. This capability provides the user with access to address the fault causing the alarm so that it could be corrected thereby stopping the alarm.

Resolving the alarm may include any action that addresses the condition that caused the alarm to be generated. For example, resolving the alarm may include replacing an insulin cartridge, changing a site where the ambulatory medicament device is connected to the subject, charging a battery of the ambulatory medicament device, providing insulin or a counter-regulatory agent to the subject and/or the ambulatory medicament device, or any other action that may be performed to address an alarm condition. In some cases, the resolution action may be acknowledging the alarm. For example, if the alarm is informational (e.g., to inform the user that more insulin has been ordered), acknowledging the alarm may be a sufficient resolution action.

In some cases, whether the alarm condition is resolved may depend on an identity of the user. For example, if a child interacts with an alarm related to reordering of insulin, the alarm may remain until a parent or guardian acknowledges the alarm. However, the child may be able to snooze the alarm. In some cases, a user interface that displays alarms may differ based on who is viewing the alarm. For example, a child may view the alarms, but may not be able to interact with the alarms. However, a parent or guardian may be able to snooze or dismiss an alarm. Further, a child may be instructed to bring the device to a parent or adult to address an alarm. In some cases, the child may be informed of how urgently to contact the parent (e.g., contact a parent immediately, within a day, within a week, etc.). Moreover, a designated adult may separately be alarmed (e.g., via a text or email alarm). The parent or guardian may receive additional information not provided to the child or subject (e.g., a link to repair instructions or a workflow to address the alarm condition).

In some cases, certain conditions may self-resolve over time. For example, a low battery alarm may resolve as the battery is charged. In such cases, the alarm may be cancelled automatically as the battery charge level exceeds a particular threshold. Further, in some cases, one or more alarms and/or the alarm list can be viewed and/or accessed on a home screen, a main screen, or other non-alarm based user interface screen in addition to a user-interface screen designated for display alarms. The alarm list may be accessed from the ambulatory medicament device and/or a computing system in communication with the ambulatory medicament device.

However, in some cases, the alarm condition may or may not be resolvable when the ambulatory medicament device is locked.

A user may interact with the alarms generated based on the alarm condition. In some cases, the user can only interact with the alarms when the AMD and/or the user interface is unlocked. In some cases, the user can interact with the alarms to snooze them or to obtain further information, when the AMD is locked. However, the user may not be able to dismiss the alarm without unlocking the ambulatory medicament device. Interacting with the alarms may include providing information associated with the alarm to a user in response to the user interacting with the alarm, or an indicator representative of the alarm.

Example AMD with Alarm Management System

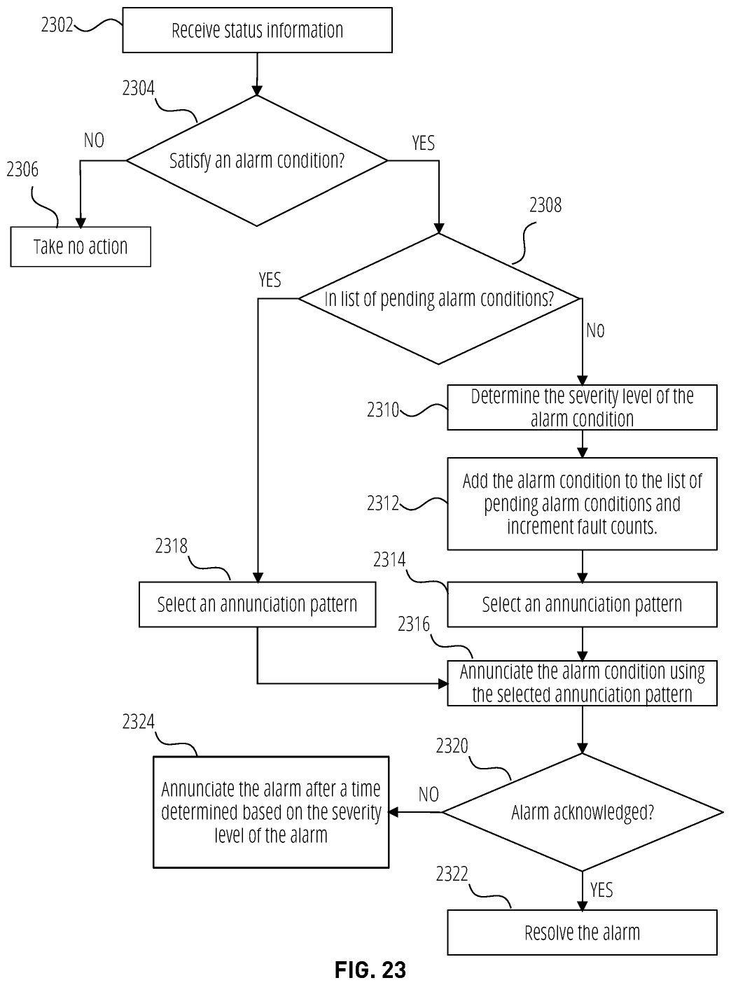

FIG. 23 shows a flow diagram illustrating an example procedure that may be used by the alarm system of an AMD to annunciate an alarm condition upon receiving a status information that satisfies an alarm condition. In some examples, the alarm annunciation and control system 1012 (deleted) implements an annunciation process by execution of instructions by a processor in CCM of the AMD, where the instructions can be stored in the main memory, storage of the AMD, or in a memory of a connected electronic device or computing system.

The alarm system may receive status information at block 2302 from one or more device sensors 1004 (deleted) and/or one or more subject sensors 1010 (deleted) via the monitoring system interface 1008 (deleted). The one or more device sensors 1004 (deleted) may include any type of sensor that can determine a condition of the AMD. For example, the one or more device sensors 1004 (deleted) may include a battery charge sensor that determines a charge of a battery, a battery condition sensor that determines a condition of a battery, a medicament sensor that determines a quantity of medicament remaining, or any other type of sensor that can determine a condition of one or more electronic or mechanical components of the AMD. The one or more device sensors may determine if a quantity of medicament is below a threshold or if a battery charge is below a threshold.

The one or more subject sensors 1010 (deleted) can include any type of sensor that can determine a health-related characteristic or physiological parameter of the subject. For example, the one or more subject sensors 1010 (deleted) can determine a blood glucose measurement, a blood pressure measurement, a respiratory rate, a blood oxygenation level, a pulse rate, or any other physiological characteristics of a subject. In particular, although not limited as such, the one or more subject sensors 1010 (deleted) may measure any physiological parameter of the subject that may relate to monitoring, managing, or treating a subject's diabetes.

In some examples, the alarm annunciation and control system 1012 (deleted) determines whether the received status information satisfies an alarm condition at decision block 2304. In some examples, the alarm condition may be an alarm condition in an alarm profile. If the received status information does not satisfy an alarm condition, no action may be taken at block 2306. If the received status information satisfies an alarm condition at decision block 2304, the alarm system may determine whether the alarm condition is already present in the list of pending alarm conditions at decision block 2308. If the alarm condition is not present in the list of pending alarm conditions, the alarm system may determine the severity level of the alarm condition at block 2310, add the alarm condition to the list of pending alarm conditions at block 2312, and increment an alarm count that tracks a number of occurrences of an alarm or a fault count that tracks a number of faults occurring in the AMD. In some examples, the placement of the alarm condition in the list of pending alarm conditions may depend on the determined severity level of the alarm condition. In some such examples, the alarm conditions may be categorized numerically in descending order with the highest priority fault displayed at the top.

Next, based on the determined level of severity, the alarm annunciation and control system 1012 (deleted) may select an annunciation pattern at block 2314 and annunciate the alarm condition using the selected annunciation pattern at block 2316. If the alarm condition is present in the list of pending alarm conditions, the alarm system may select an annunciation pattern at block 2318 and annunciate the alarm condition using the selected annunciation pattern at the block 2316. In some examples, the annunciation pattern selected at block 2318, may be an annunciation pattern that is different than the previously used annunciation patterns for the alarm condition. In some such examples, the annunciation pattern selected at block 2318, may be selected based at least in part on a number of times that the same alarm condition is satisfied by a received status information. The process of the alarm detection and control function may repeat periodically, intermittently, according to a particular schedule, or while the ambulatory medical device is in use. The frequency with which the process is repeated may depend on the particular alarm condition detected from the status information. In some examples, after an alarm is annunciated, the alarm system may wait for user acknowledgment of the alarm at decision block 2320. If the user acknowledges the alarm, the system proceeds to resolve the alarm at block 2322. In some cases, resolving the alarm may include providing instructions to a user or indicating where a user can locate instructions to resolve the alarm condition. For example, the user may be provided with repair instructions for repairing the AMD. Further, in some cases, resolving the alarm may include automatically ordering or requesting that the user confirm an order is to be placed to replenish a medicament. If the user fails to acknowledge the alarm, the annunciation may be repeated after a certain time period at block 2324 determined based on the severity level of the alarm. In some examples, if the user fails to acknowledge the alarm, the annunciation continues and may escalate depending on the severity level of the alarm.

As mentioned above, the alarm conditions may be categorized based and annunciated based on their severity level. In some examples, the alarms are categorized numerically in descending order with the highest priority fault displayed at the top of the list. In some examples, a level 0 severity, may be for a trivial fault that does not require any action by the user thus not warranting an alarm notification. In some examples, a level 1 severity may be an informational type notification that repeats at a certain frequency (e.g. every 30 minutes) until acknowledged by the user which results in it being reset. The annunciation may include a brief vibration and a beep, for example. In some examples, a level 2 severity, may be one relating to an imminent loss of system function. Thus, such an annunciation may include two brief vibrations and two beeps, for example, and repeating at a certain frequency (e.g. every 30 minutes). Thus, the user would still need to address the situation creating the fault to completely stop the annunciation. In some examples, a level 3 fault may be for when the system is no longer fully functional thus requiring user intervention to correct the issue. The annunciation may begin with a base level intensity with three brief vibrations and three audio beeps, for example, and repeating at a certain frequency (e.g. every 5 minutes). The annunciation escalates at a second frequency, e.g. every 30 minutes, up to a maximum intensity level. The escalation may be a change in vibration intensity and/or audio level, for example. The escalation may be cleared to a base level when the user acknowledges the fault; however, the base alarm may remain if the underlying condition persists. Thus, the user would still need to address the situation creating the fault to completely stop the annunciation. In some examples, a level 4 severity, may be for when the system is no longer functional and not correctable by the user. The annunciation may begin with a base level intensity with three audio beeps, for example, and repeating at a certain frequency (e.g. every 5 minutes). The annunciation escalates at a second frequency, e.g. every 30 minutes, up to a maximum intensity level. The escalation may be a change in audio level, for example. The escalation may be cleared when the user acknowledges the fault; however, the base alarm remains because the underlying condition persists. In some examples, a level 5 severity, may be for high priority alarms per IEC 60601-1-8. The annunciation when activated may be cleared only when the underlying issue is resolved, e.g., glucose level is raised.

Additional embodiments relating to determining a severity of an alarm condition and annunciating the alarm based at least in part on the severity of the alarm condition that can be combined with one or more embodiments of the present disclosure are described in U.S. Provisional Application No. 62/911,017, which was filed on Oct. 4, 2019 and is titled “ALARM SYSTEM AND METHOD IN A DRUG INFUSION DEVICE,” the disclosure of which is hereby incorporated by reference in its entirety herein for all purposes.

### Non-Critical AMD Condition Management

In some cases, a condition may occur that impacts the operation of the ambulatory medical device (AMD) that provides therapy to a subject. In some examples, an AMD can be an ambulatory medicament device. This condition may be associated with the ability of the AMD to operate as intended by the manufacturer, a subject receiving therapy from the AMD, and/or user (e.g., healthcare provider, parent, or guardian of the subject). In some cases, the condition or malfunction of the AMD may prevent the AMD from providing therapy to the subject. In some cases, the condition or malfunction may permit, at least for a period of time, the AMD to continue providing at least partial therapy to the subject. It is generally desirable to generate an alert to inform the subject and/or one or more users of the condition of the AMD and/or the subject to provide and facilitate non-critical malfunction management in an ambulatory medical device. Moreover, it is desirable to track the alert until the condition that caused the alert is resolved. Further, it is desirable to issue different types of alerts for different conditions to enable a subject or user to easily distinguish the severity of the condition that triggered the alert.

In many cases, if the nature of the alert is non-critical, it may be safer to continue the underlying therapy and notify the user of the condition than to cease therapy. In some such cases, the best response to a problem with the device for a subject is to notify the device manufacturer, or other user that can address the problem, while the subject continues to receive therapy until a replacement device can be obtained or a repair can be made.

Additionally, alert fatigue can be an issue with medical devices due to excessive alerts which do not necessarily require user interaction. Alert fatigue can be dangerous because it can lead users to ignore serious alerts or alerts that require action in the short term.

The method described herein may be performed by an AMD (e.g., by one or more processor of the AMD) to detect device malfunctions for the AMD and that can generate alerts corresponding to the AMD and prioritize the alerts to enable the subject or user to quickly and easily determine whether the device malfunction will impact therapy, should be addressed in the short-term (e.g., immediately, in 1-2 hours, within the day, etc.), and/or can be addressed at the subject's convenience (e.g., within a month, or more). In some cases, the method of device malfunction alert prioritization may be used by other systems.

In certain embodiments, the system disclosed herein can detect a condition in which the AMD does not meet a manufacturer's specification (e.g. a failure of a haptic annunciator, a Bluetooth® radio malfunction, glucagon or insulin runs out, there is a medicament delivery malfunction, a touchscreen failure, etc.). In some cases, there can be several tiers of critical and/or non-critical faults. If it is determined that the underlying condition is not sufficient to stop therapy (e.g., stop delivery of insulin), the fault may be deemed non-critical. In some cases, the fault may not be a fault of the device, but may be indicative of required maintenance (e.g., recharge battery indicator, order more medicament indicator, etc.). The condition may be annunciated to the user with appropriate instructions (e.g., call manufacturer for replacement medicament or parts) for addressing the fault or issue.

After the annunciation is acknowledged, the alert may be re-annunciated, or annunciated again, as a reminder at some later period in time (e.g. the alert may be re-annunciated daily at 4:00 PM or on Saturdays at noon providing, for example, for a fixed static time period or periodically between alerts). The length of time between annunciations may depend on the severity of the fault. In some cases, the re-annunciation cannot be stopped by the user, but may only cease if the underlying condition is resolved. In some cases, the re-annunciation time period may be a variable time period and may gradually increase to minimize fatigue alert. In some cases, the re-annunciation time period may be a variable time period and may gradually decrease if the alert becomes more urgent or increase in urgency. In some cases, the re-annunciation time period may change during the day based on time of day. For example, alerts may be provided during the day, but silenced or reduced during the night.

The method may include detecting a condition of the AMD. In some examples, the condition of the AMD may comprise a set of operating parameters of the AMD. In some such examples, the condition of the AMD may be determined using one or more sensors of the AMD. Further, the condition of the AMD may be determined by the presence or absence of one or more errors when performing one or more functions of the AMD. For example, if the AMD fails to establish a communication connection with a control system or a data storage system, it may be determined that there is a possible malfunction with the AMD. As another example, if the AMD fails to deliver medicament or detects an error when attempting to deliver medicament, there may be a malfunction with the medicament pump. In some cases, the condition of the AMD may be determined based on one or more configuration values being outside a normal operating range. For example, if the speed of delivery of medicament is faster or slower than a configured operating range, then it may be determined that there is a malfunction with the medicament pump or a connection with a medicament delivery tube (e.g., a catheter). In some examples, the condition of the AMD may be determined based on the performance of the AMD over period.

The method may include comparing the detected condition of the AMD to a set of normal operating parameters. In some examples, the set of normal operating parameters may be the specifications set by the manufacturer for when the AMD is operating as intended by the manufacturer. In some examples, at least some of the normal operating parameters may be provided by a healthcare provider. In some examples, at least some of the normal operating parameters may be provided by the subject or an authorized user. In some cases, the normal operating parameters may be associated with a range of values. For example, the operating parameter for a speed of medicament delivery may be associated with a range of speeds, which may vary based on user settings, medicament type, site location of medicament delivery, or manufacturing tolerances, among other parameters. Comparing the detected condition of the AMD to the set of normal operating parameters may include comparing each operating parameter in the specification to a corresponding detected operating parameter of the AMD. The AMD may generate a user alert or non-critical malfunction alert based on the determined condition of the AMD. For example, the AMD may generate an alert when the detected condition of the AMD does not satisfy a set of normal operating parameters.

The method may further include determining whether the detected condition satisfies a minimum set of operating parameters. In some cases, the minimum set of operating parameters may match the normal operating parameters. However, typically the minimum set of operating parameters differ from the normal operating parameters. The minimum operating parameters may include the minimum specifications, minimum parameters, or minimum condition required by the AMD to maintain or continue providing therapy to the subject. In other words, the minimum operating parameters are the operating parameters sufficient to provide therapy. However, the minimum operating parameters may not be sufficient to enable all features of the AMD. For example, the minimum operating parameters may permit the AMD to deliver insulin to the subject, but may not be sufficient to deliver the insulin at a normal delivery speed for the particular AMD. As another example, the minimum operating parameters may permit the delivery of therapy, but may not be sufficient to track a log of therapy or to transmit a therapy log to another computing system. In some cases, the normal operating parameters and/or minimum operating parameters may be specified by be specified by the manufacturer at the time of manufacturing. In some some cases the normal operating parameters and/or minimum operating parameters may be specified by a subject or healthcare provider (e.g., the minimum amount of medicament that is to be provided in each bolus may be specified by a healthcare provider). In some cases, the normal or minimum operating parameters may be modified.

When it is determined that the condition of the AMD satisfies at least the minimum operating parameters, the AMD may be configured to maintain delivery of therapy to the subject. Maintaining delivery of therapy may include maintaining therapy at the same rate, at a reduced rate (e.g., providing only basal therapy and therapy responsive to a meal announcement), or at a minimum rate or minimum maintenance rate (e.g., providing only basal insulin). Advantageously, the ability of the AMD to distinguish between a minimum set of operating parameters and a normal set of operating parameters enables an AMD with a malfunction to continue providing therapy, which sometimes includes life-saving treatment, to a subject until the AMD can be repaired or until the condition of the device worsens to a point where the minimum operating parameters cannot be maintained. In some cases, the AMD may temporarily maintain delivery of therapy. Temporarily maintaining therapy may provide a subject time to address the issue that caused the AMD to not satisfy the normal operating parameters before the subject loses access to therapy. In some cases, the AMD temporarily maintains therapy until the device condition makes it no longer possible to maintain therapy.

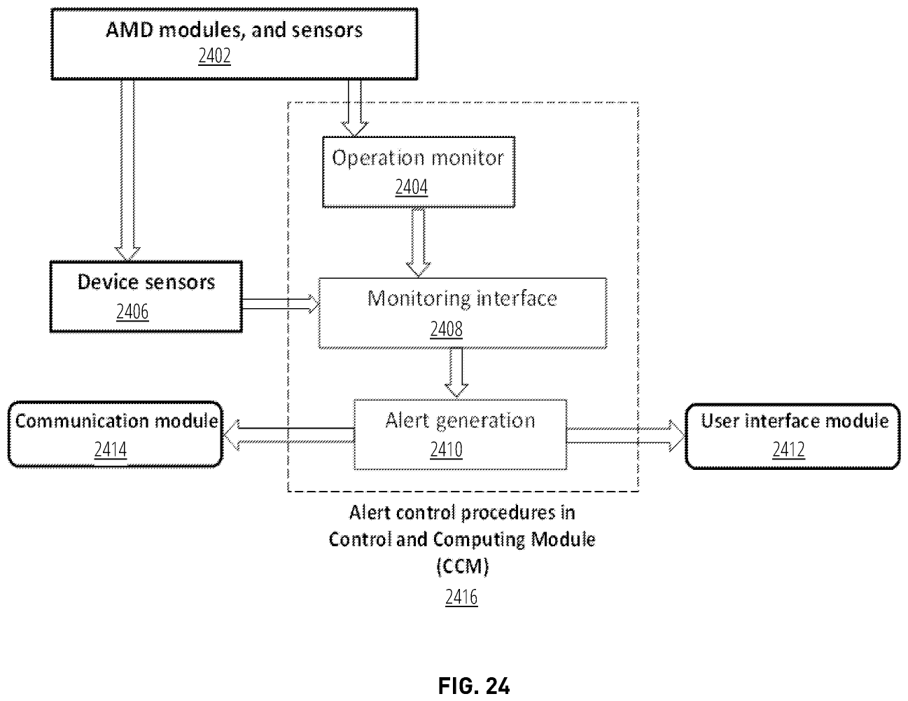

FIG. 24 is a block diagram illustrating an example of the interconnection among modules and procedures in AMD that may be involved in monitoring the condition of the AMD and generating an alert when a device malfunction is detected. In some examples, the condition of AMD may include the status of the modules and components of the AMD and/or operation of modules and procedures of the AMD. In some embodiments, the alert system may be implemented as a set of alert control procedures 2416 in the control and computing module 610 (CCM) of the AMD. The alert control procedures 2416, may be implemented as instructions stored in a memory of AMD (e.g., a memory in CCM 610) and executed by a hardware processor 614 of the AMD (e.g., a processor in CCM 610) to generate an alert upon detection of a malfunction of the AMD. In some cases, the hardware processor may be a hardware processor of the AMD that controls medicament delivery. In some cases, the hardware processor of the monitoring system may be a separate hardware processor.

In some examples, the alert control procedures 2416 may include a monitoring interface 2408, a set of operation monitoring procedures 2404 and a set of alert generation procedures 2410. In some examples, a set of device sensors 2406 may be configured to track the status of the components of the AMD. A set of operation monitoring procedures 2404 may be configured to monitor the operation of the components, modules and other procedures (e.g., temporal behavior of the signals provided by the components, communication between different devices and modules, performance of the procedures implemented in the CCM 610 and the like). For example, a device sensor may determine that a component is properly connected and it is functional, while the operation monitoring procedures 2404 may provide data related to signals generated by the component over a period. The monitoring interface 2408 may monitor and evaluate a set of operating parameters of the AMD at least partially based on the information received from the operation monitoring procedures 2404 and device sensors 2406.

In some embodiments, the alert generation procedures 2410 may compare the determined operating parameters of the AMD, received from the monitoring interface 2408, with a set of normal operating parameters. In some examples, the alert generation procedures 2410 may also determine whether the operating parameters of the AMD satisfies a minimum set of operating parameters. In some examples, if it is determined that one or more operating parameters of the AMD do not satisfy the normal operating parameters, the alert generation procedures 2410 may generate an alert. In some examples, the alert may be transmitted to the user interface module 2412 and displayed on a display of the AMD (e.g., a touchscreen display). In some examples, once an alert is generated the AMD may establish a connection (e.g., a wireless connection) with another device using the communication module 2414. This other device may include a local device (e.g., a laptop, smartphone or smartwatch of the user) or a computing system of a cloud-based service. In some such examples, the alert may be transmitted by the communication module 2414 to the local device and/or the computing systems where it may be displayed on user interface associated with the local device or the computing system. In some cases, the local devices and/or the computing system may receive data from the AMD device enabling the user to monitor the operating parameters of the AMD.

The type of the alert, and the frequency at which the alert is repeated, or whether an alert is dismissible or not, may be determined by the alert generation procedure based on the detected condition of the AMD and the alert information stored in a memory of the AMD. In some examples, the alert information may be provided by the subject, an authorized user or a healthcare provider. In some examples, the alert information may be stored in the AMD at time of manufacturing.

In some examples, upon determination that the detected AMD condition (e.g., comprising a set of operating parameters) does not satisfy a normal condition (e.g., a set of normal operating parameters), the alert generation procedures 2410 may cause the medicament delivery interface 606 to stop therapy delivery or modify one or more delivery parameters (e.g., therapy delivery rate). In some examples, upon determination that the detected or determined that the operational parameters of AMD do not satisfy a set of normal operating parameters, but satisfy a set of minimum operating parameters, the therapy delivery may be maintained at a normal rate.

The alert may include any type of alert. For example, the alert may be a visual alert (e.g., a light or changing light), an audible alert (e.g., a beep or series of beeps), a haptic or vibration alert, an email alert, a text alert, or any other type of alert. In some examples, different AMD conditions or different operational parameters of the AMD may be associated with or may trigger different types of alerts. Thus, the alert may enable the user to determine the device condition of the AMD based on the type of alert. For example, an indication that the AMD failed to deliver a medicament may trigger one type of alert while an indication that the level of a medicament in the AMD has dropped below a particular level may trigger a different type of alert. In some cases, the user alert or non-critical malfunction alert is dismissible and/or may be snoozed by the user. In some cases, such as when the AMD fails to satisfy a set of minimum operating parameters, the user alert or non-critical malfunction alert may not be dismissible or cannot be snoozed.

A dismissible alert may be scheduled to repeat on a particular schedule until an alert modification condition occurs. The frequency with which the dismissible alert repeats may depend on the severity of the condition or the particular operating parameters that do not satisfy normal or minimum operating parameters. More urgent device conditions may result in alerts that repeat more frequently. Further, alerts may vary based on when the condition was detected, the time of day, or the detected activity of a subject (e.g., sleep, abnormal activity, or elevated activity, such as exercise). Similarly, the snooze options may vary for different alerts or any of the aforementioned conditions. In some cases, the AMD may escalate or prioritize an alert if it detects that the condition of the AMD has become more critical. In some cases, the re-annunciation time period or variable time period may gradually increase to minimize fatigue alert, or the condition of the AMD has become less critical. In some cases, the re-annunciation time period or variable time period may gradually decrease if the condition of the AMD has become more critical.

The alert frequency may be for a static time period (e.g., every 5 hours) or may ramp towards more frequency (e.g., every 5 hours for 1 to 3 reminders, every 4 hours for 3 to 6 reminders, etc.), or may change based on time of day (e.g., snooze alerts during sleeping hours for non-urgent alerts), etc.

In some examples, the alert modification condition may include any action that causes the operating parameters of the AMD to return to normal operating parameters. For example, the alert modification condition may be a repair or replacement of a faulty component. In some cases, the alert modification condition may be an acknowledgement of the alert. In some examples, the alert modification condition may include a worsening of the AMD condition. In such cases, the modification to the alert may include the substitution or prioritization of the alert to a different alert that indicates a different or more serious condition of the AMD. For example, an urgent condition may become critical if the detected malfunction is not addressed after generating certain number of alerts. When an urgent condition becomes critical, it may be prioritized, trigger a different alert types or different user/non-critical malfunction alert types (e.g., a louder sound, different sound, different frequency, brighter image or the like), and/or escalation in the alert frequency. For example, the audible alert may become louder and may be combined with a vibration alert from a haptic annunciator. Moreover, if the condition reaches a critical state, the AMD may cease providing therapy to the subject.

In some cases, generating the alert may further include contacting a manufacturer and/or healthcare provider (e.g., clinician). Further, generating the alert may include ordering replacement parts. In some cases, the alert may instruct a subject or user on how to repair the ambulatory medical device.

Once the malfunction is addressed, the AMD is repaired, or the condition that caused the alert is resolved, a user may permanently (or until the next time a device condition triggers the alert) dismiss the alert. Alternatively, or in addition, the AMD may automatically dismiss the alert when it determines that the device condition that caused the alert has been resolved (e.g., using the alert control procedures 2416). In some cases, the AMD may periodically recheck the device condition to determine whether the alert condition has been resolved.

In some cases, the manufacturer or healthcare provider may remotely clear or stop an alert using, for example, using a device or computing systems that is connected to the AMD (e.g., via a wireless connection such as an NB-LTE connection). In some cases, only the manufacturer and/or healthcare provider can clear or stop the alert. Further, in some cases, a manufacturer and/or a healthcare provider may notify a user (e.g., a subject, or parent or guardian) of an issue or impending issue with the AMD. The notification may be received by the AMD device via a wireless connection (e.g., NB-LTE connection). Alternatively, or in addition, the notification may be received via a computing device, such as a smartphone or laptop.

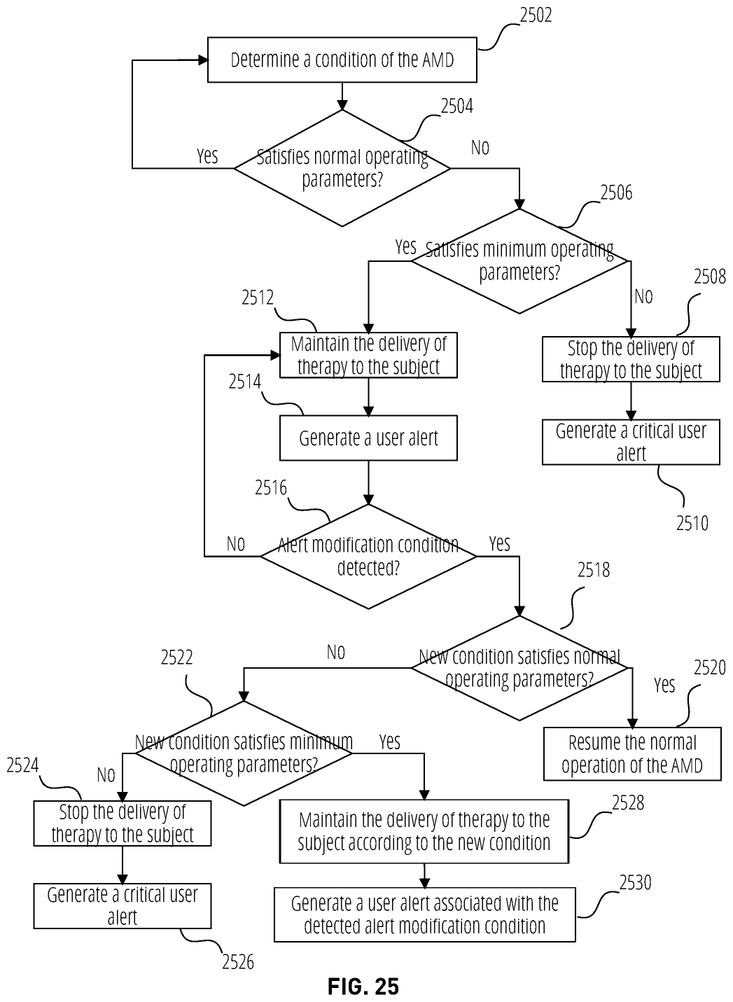

FIG. 25 is a flow diagram illustrating an example procedure that may be used by the alert system of an AMD to monitor the operation of an AMD and generate alerts when a device malfunction is detected. In some examples, the alert system continuously monitors the status of all modules and components associated with AMD as well as the operation of all modules and procedures of the AMD. When a condition of the AMD is detected or determined at block 2502, the alert system may determine whether the detected device condition satisfies a set of normal operating parameters at the decision block 2504. If it is determined that the detected device condition satisfies a set of normal operating parameters, the alert system takes no action and continuous monitoring the AMD at block 2502.

If it is determined that the device condition does not satisfy a set of normal operating parameters, the alert system determines whether the detected device condition satisfies a set of minimum operating parameters at decision block 2506. If, at decision block 2506, it is determined that the device condition does not satisfy a set of minimum operating parameters, the alert system may send a signal to the therapy delivery module 606 or medicament delivery interface 1004 to stop delivery of therapy to the subject at block 2508, and to generate a critical user alert or critical alert at block 2510 that is prioritized by indicating that immediate or urgent action is required. In some examples, upon generation of a critical alert the alarm system of the AMD, may contact a healthcare provider or certified user (e.g., parent or guardian of the subject) and also send the critical alert to one or more computing devices (e.g., laptop, cell phone, personal computer, and the like) of the healthcare provider or certified user.

If, at decision block 2506, it is determined that the device condition satisfies a set of minimum operating parameters, the alert system may maintain the delivery of therapy to the subject at block 2512 and generate a user alert or non-critical malfunction alert at block 2514. In some such examples, the alert system may maintain the delivery of the therapy at rate associated with the detected condition of the AMD (e.g., normal rate or minimum maintenance rate) until an alert modification condition is detected at decision block 2516.

Upon detection of an alert modification condition at decision block 2516, the alert system may determine whether the new device condition satisfies a normal set of parameters at decision block 2518. If, at decision block 2518, it is determined that the new device condition satisfies a set of normal operating parameters, the alert system may resume the normal operation of the AMD at block 2520 (e.g., deliver the therapy at a normal rate). If at decision block 2518, it is determined that the new device condition does not satisfy a set of normal operating parameters, the alert system may determine whether the new device condition satisfies a minimum set of parameters at decision block 2522. If, at decision block 2522, it is determined that the new device condition satisfies a set of minimum operating parameters. The alert system may maintain or modify the rate of therapy delivery according to the new device condition at block 2528 and generate a user alert or non-critical malfunction alert at block 2530 according to the new device condition. If, at decision block 2522, it is determined that the new device condition does not satisfy a set of minimum operating parameters, the alert system may send a signal to the therapy delivery module to stop delivery of therapy to the subject at block 2524, and generate a critical user alert at block 2526 indicating that immediate or urgent action is required. The critical user alert may be prioritized over other types of alerts and alarms. In some examples, upon generation of a critical alert the alarm system of the AMD, may contact a healthcare provider or certified user (e.g., parent or guardian of the subject) and also send the critical alert to one or more computing devices (e.g., laptop, cell phone, personal computer, and the like) of the healthcare provider or certified user.

### Pump Replacement

It may be useful to have a control system, which may be part of an ambulatory medicament pump or other medicament device, to transmit information related to the functionality of the ambulatory medicament pump to a remote computing device. For example, if the ambulatory medicament pump begins to malfunction or in some other way fails to meet a threshold level of functionality, this could impact the safety of the ambulatory medicament pump. For example, the system may identify that the system fails to meet a threshold manufacturer specification (e.g., a failure of a haptic annunciator, a Bluetooth® radio malfunction, glucagon or insulin runs out, there is a medicament delivery malfunction, a touchscreen failure, etc.). The control system may be able to automatically detect the malfunction and provide an alarm to the user or to a remote computing device. In some embodiments the control system can cause the ambulatory medicament pump to reduce operability on a temporary or permanent basis. For example, the ambulatory medicament pump may be shut down until the malfunction has been repaired.

It can be beneficial for an ambulatory medicament device to function properly. As discussed herein, an improperly functioning ambulatory medicament pump can be dangerous for a subject. Accordingly, a control system that can monitor the ambulatory medicament pump and/or provide alerts when the ambulatory medicament pump or one or more parts thereof require replacement. It may be particularly beneficial to have the control system automatically send a request for the replacement part/pump, such as via user interaction with a user interface. It may be beneficial to provide additional functionality as well.

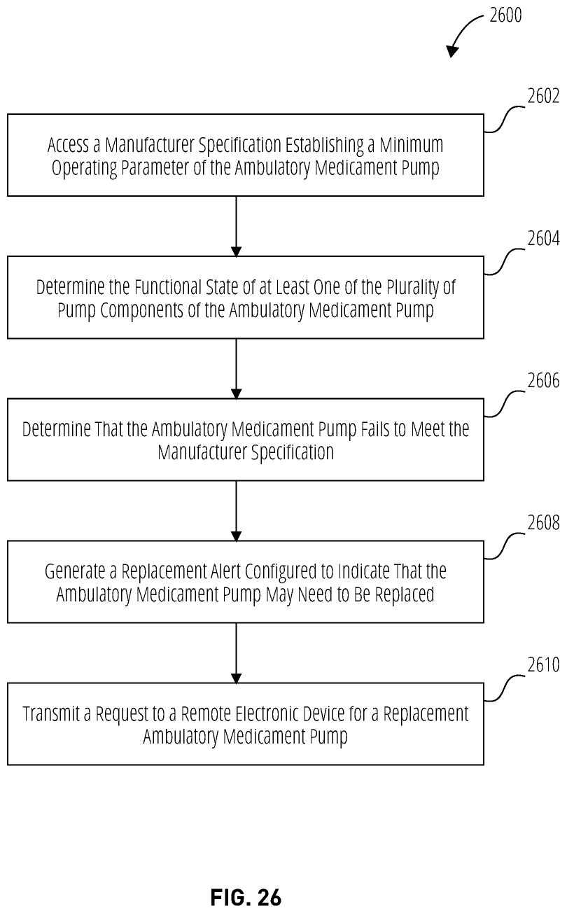

FIG. 26 shows a flow diagram illustrating an example method 2600 that may be used by a control system, such as one described herein (e.g., the glucose control system 308, the glucose control system 200b, the AMP 600, the glucose level control system 1102), to determine that the ambulatory medicament pump fails to meet a manufacturer specification and to transmit a request for a replacement ambulatory medicament pump or other ambulatory medicament device (e.g., AMD 100, AMD 202, AMP 600, AMP 702, ambulatory medical device 500, etc.).

At block 2602 the control system can access a manufacturer specification configured to establish a minimum operating parameter of an ambulatory medicament pump. The control system may be in electronic communication with the ambulatory medicament pump, such as via one or more data interfaces. In some embodiments the one or more data interfaces includes a wireless data interface. The data interface may be any data interface described herein. In some embodiments, the data interface may be or consist of software instructions. The manufacturer specification can indicate a minimum or maximum threshold outside ether of which the control system can identify a potential problem or issue that may need to be addressed.

At block 2604 the control system may determine the functional state of the at least one of the plurality of pump components of the ambulatory medicament pump. Determining the functional state of pump component(s) can include automatically receiving the functional state via the pump monitoring system. In some embodiments, the functional state may be received from a remote electronic device, such as a remote computing environment (e.g., the “cloud”). The remote computing environment may be configured to track the functional state of the ambulatory medicament pump over time (e.g., by receiving updates transmitted wirelessly via a data interface). The plurality of pump components can include one or more of a user interface, a battery, a charging element, a power management controller, a medicament reservoir, a wireless interface, a pump controller, and/or a pump motor. The determination may be made via a pump monitoring system associated with the ambulatory medicament pump. The pump monitoring system may be coupled to the ambulatory medicament pump in some embodiments. The pump monitoring system may include one or more sensors configured to track one or more performance metrics of the ambulatory medicament pump. For example, the pump monitoring system can include an electrical sensor that tracks a charge and/or discharge rate of the battery. The pump monitoring system can include a sensor that tracks the output of a user interface associated with the ambulatory medicament pump, such as one described herein. The pump monitoring system can include a resistive sensor to determine whether a mechanical failure has occurred (e.g., a pump failure, an improper fluid pressure indicative of a fluid leak or pressure spike, etc.). The pump monitoring system may include software instructions configured to identify a software error or malfunction.

The functional state of the pump components that the control system may identify can include a variety of functional states. The functional state may be indicative of a functional ambulatory medicament pump. Additionally or alternatively, the functional state may be indicative of a non-functional or sub-optimal state of the ambulatory medicament pump. For example, the functional state may include a battery failing to meet a manufacturer's specification, an input/output communication error, an electrical failure, a mechanical failure, a fluid pressure outside a pressure threshold, a pump controller malfunction, an error associated with the non-transitory memory, the pump has exceeded a manufacturer's warranty, a software malfunction, the pump has an indication of being tampered with, the pump has exceeded a usage threshold criterion, the pump is subject to a manufacturer recall, and/or some other similar functional state. The functional state may correspond to an error discussed above, which may be associated with an alarm of a particular severity.

As noted above, the severity level of the error or alarm may be associated with how urgently the condition or malfunction should be addressed or resolved. Additionally or alternatively, the severity level may be associated with an amount of harm that may be caused to a subject if the condition that triggered the alarm is not resolved or is not resolved within a particular time period. The number of severity levels may be limited to a particular number, such as 3, 5, 6, 9, or some number in between. However, it is possible for there to be more than 9 severity levels. Additional details related to the discussion of alarm conditions above can be applied to the functional states identified here. In order to avoid unnecessary repetition, those details are not repeated here, but can be applied as applicable.

At block 2606 the control system can determine that the ambulatory medicament pump fails to meet the manufacturer specification. The determination may be in response to determining the functional state of the at least one of the plurality of pump components, for example if the functional state is one that requires attention and/or needs to be resolved. The determination may be based additionally or alternatively on a severity level of the malfunction.

In response to determining that the ambulatory medicament pump fails to meet the manufacturer specification, the control system may take one or more actions. In some embodiments, the control system may maintain the delivery of therapy by the ambulatory medical device to the subject, for example if the error or malfunction is not critical and/or related to safe delivery of treatment to a subject. For example, the control system may not prevent the AMD from providing therapy so long as the AMD is able to provide therapy. However, in some examples, if the functional state or other condition interferes with the delivery of therapy, operation of the AMD may be suspended temporarily or permanently. Additionally or alternatively, the rate of the medicament delivery may be reduced (e.g., only basal insulin, higher threshold for delivering insulin boluses, etc.), such as when the functional state is of a medium concern and/or related to a user error. Generally, if the control system reduces therapy based on the functional state, then the functional state may be associated with a higher severity level. However, some functional conditions that do interfere with the provisioning of therapy may be associated with lower severity levels. For example, a determination that the AMD cannot supply insulin may normally be associated with a most critical functional state that requires immediate action (e.g., alarm, termination of ambulatory medicament pump functionality, etc.).

In response to determining that the medicament pump fails to meet the manufacturer specification, the control system can automatically generate a replacement alert. The replacement alert can indicate that the ambulatory medicament pump may need to be replaced, depending on the severity of the failure to meet the manufacturer specification as described herein. The replacement alert may be delivered manually via user interaction with a pump replacement control element (e.g., one of the user interfaces described herein). The replacement alert can be annunciated in any way described herein, such as a video, audio, haptic, or other form of annunciation.

In some embodiments, the control system may determine that alarm condition is associated with a lower severity level (e.g., an informational alarm reminding the user that insulin cannot be delivered during a site change, lack of proper charging of battery, user error associated with the ambulatory medicament pump, etc.). In some examples, in response to determining that the severity level of the alarm condition matches an unsafe operation (e.g., a condition that may cause the AMD to provide doses of the medicament that are above or below certain values, or to unreliably determine the subject's condition), the AMD may suspend delivery of the medicament to the subject. Once the condition is resolved, the AMD may resume delivery of medicament to the subject for a period of time. The period of time can include a preset period of time, a selected period of time, or some other option. The period of time may be determined at least in part on the severity and/or seriousness of the alarm condition. If on the other hand it is determined that the alarm condition matches a safe operation severity level, the AMD may be configured to maintain delivery of medicament to the subject. The control system may repeat generation of the replacement alert on a schedule until an alert modification condition occurs, such as one described herein. Other aspects of the delivery of the alert can include any aspect discussed above with respect to alert delivery (e.g., timing, scheduling, annunciation pattern, threshold(s), etc.).

The replacement alert may be accessible by a user. The user may be able to provide a request to send a replacement pump and/or replacement pump part in response to the replacement alert. For example, the control system may receive, via a user interface described herein, a selection of the request (e.g., via a touch interface). In some embodiments, the control system can confirm the selection. The confirmation may also be via the user interface and may include a question. The question may require the user to present credentials confirming the user's identity and/or knowledge with respect to the ambulatory medicament pump and/or the subject receiving therapy. For example, personal identifying information may be required and/or a secret passcode. In some embodiments, in response to determining that the medicament pump fails to meet the manufacturer specification, the control system may prompt the subject to authorize a replacement of the ambulatory medicament pump. The control system may receive (e.g., via the pump replacement control element) a command to authorize the replacement of the ambulatory medicament pump. Other variants described herein within to security may be present depending on the embodiment.

In some embodiments, the control system may, in response to determining that the medicament pump fails to meet the manufacturer specification, prompt the subject to select a time period for which the replacement of the ambulatory medicament pump is desired. The selection may be via a graphical user interface described herein, which may include the pump replacement control element. The control system may receive a selected time period for which the replacement of the ambulatory medicament pump is desired. For example, the user may select the time period, which may be a range of days, weeks, or months. This selection can provide the user more control of when the replacement part(s) are to be received. In response to receiving the selected time period for which the additional supply of medicament is desired, the control system can transmit the selected time period to one or more remote electronic devices, such as at a manufacturer or doctor.

If the system determines that the pump replacement request should be transmitted (e.g., after verification of the user credentials, in response to a user selection, automatically based on certain thresholds, etc.), the control system can transmit, via the data interface, the request to a remote electronic device for the replacement ambulatory medicament pump. The transmission may be automatic, instant, delayed, and/or according to a schedule set by a user. In some embodiments, generating the replacement alert can include contacting a third party, such as a manufacturer or a healthcare provider associated with the subject.

In some embodiments, the functional state is additionally or alternatively transmitted to the remote electronic device. This transmission can alert necessary personnel of a level of urgency and/or emergency, if any, that is associated with the replacement request. The control system may be configured to receive an indication of a status of a delivery of the replacement of the ambulatory medicament pump. This status indication may be delivered via a user interface described herein.

In some cases, the alert may be modified based on a change in the status of the ambulatory medicament pump. For example, after one or more notifications, the user may take steps to mitigate the cause of the alert. Additionally or alternatively, the modification may be a natural (e.g., automatic) change in the functional state of the ambulatory medicament pump. Such an alert medication can cause the alert to be dismissed manually or automatically, such as if the functional state is no longer beyond a threshold that triggered the alert in the first place.

In some embodiments, the control system can receive (e.g., via the data interface), a confirmation signal from the remote electronic device. The confirmation signal may indicate that the request for the replacement ambulatory medicament pump was received by the remote electronic device.

The control system may generate (e.g., via a pump replacement control element) a success alert configured to indicate that the request for the replacement ambulatory medicament pump was successfully received by the remote electronic device. This confirmation may reassure a user that the request has been properly received. The pump replacement alert and/or the success alert may be provided via any user interface described herein, such as the pump replacement control element, which can include a graphical user interface.

### Training Content Generation

Because medical devices such as ambulatory medicament devices have so much influence over the health of a subject, and because ambulatory medicament devices are becoming more complex and sophisticated, it can be beneficial to a subject's health for a user to operate the ambulatory medicament device in a proper way. It may be useful to have a control system (e.g., a pump use training system), which may be part of an ambulatory medicament pump or other medicament device, that can track user behavior relative to the determine that a use state satisfies at least one of a plurality of user interaction criteria and to automatically generate a user alert comprising a link to training content.

A system that can monitor the use of the ambulatory medicament device and provide training content to a user can help ensure that the ambulatory medicament device is properly used and therefore maintain and/or improve care to the patient via the ambulatory medicament device. A system may be able to monitor the status of the ambulatory medicament pump to determine if and/or when a replacement pump or pump part is required. This can aid a user in avoiding a lapse in therapy by helping ensure that a suitable pump and/or pump parts are available for use by the user (e.g., subject).

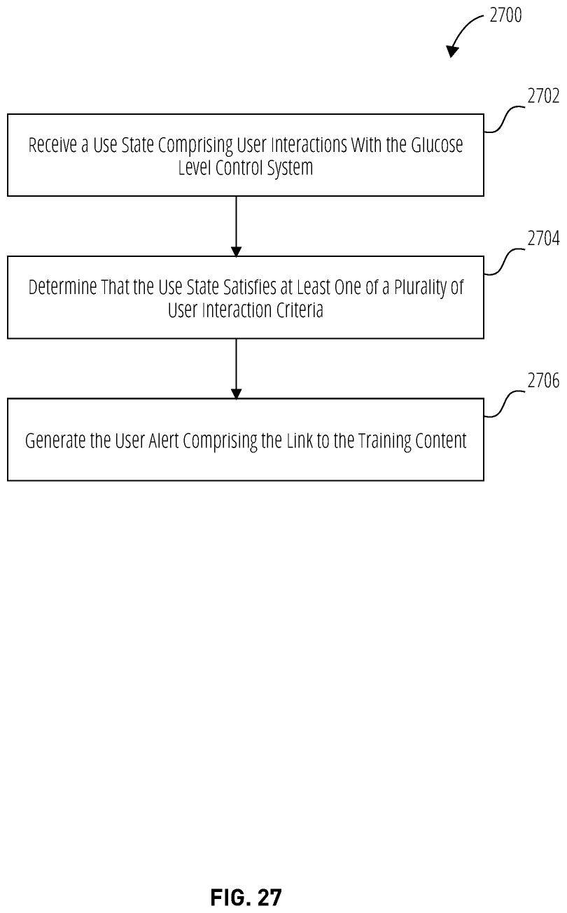

FIG. 27 shows a flow diagram illustrating an example method 2700 that may be used by a control system, such as one described herein (e.g., the glucose control system 308, the glucose control system 200b, the AMP 600, the glucose level control system 1102) to determine that a use state of an ambulatory medicament device (e.g., AMD 100, AMD 202, AMP 600, AMP 702, ambulatory medical device 500, etc.) satisfies at least one of a plurality of user interaction criteria and to automatically generate a user alert comprising a link to training content.

At block 2702 the control system can receive a use state comprising user interactions with the glucose level control system. The receipt of the use state may be via the data interface. The control system may be in electronic communication with an ambulatory medicament pump, such as via one or more data interfaces. In some embodiments the one or more data interfaces includes a wireless data interface. The data interface may be any data interface described herein. The use state can refer to how a user interacts with the ambulatory medicament pump. The use state can include one or more variables associated with the use of the ambulatory medicament pump. For example, the use state can relate to a how a user interacts with a user interface of the ambulatory medicament pump (e.g., a meal announcement control interface, a therapy change control interface, a pump settings interface, a glucose sensor replacement interface, a cartridge replacement interface). Misuse of the therapy control interface can include over-making of therapy changes, misuse of a G-burst associated with the therapy control interface, over-modification of control parameters, excessive correction boluses, excessive treatment parameter modification, and/or excessive dosing feature modification or use.

Additionally or alternatively, the use state may relate to a frequency and/or nature of one or more malfunction status alerts, such as malfunction alerts described above. The use state can relate to a frequency and/or duration of a certain threshold of battery level (e.g., a low battery level frequency) and/or of medicament level being below or above a threshold level. In some embodiments, the use state can relate to a frequency of an occlusion state within the ambulatory medicament pump. The occlusion state can refer to a level of interference of medicament delivery to the subject by the ambulatory medicament pump. The use state can relate to a level and/or duration of a glucose level signal being above or below a threshold availability frequency and/or of the glucose level signal being available below or above a threshold availability frequency.

At block 2704 the control system may determine that the use state satisfies at least one of a plurality of user interaction criteria. The user interaction criteria can relate to whether the use state is indicative of proper use of the ambulatory medicament pump. An indication that the user may be using the ambulatory medicament pump improperly may be based at least in part on a frequency and/or duration of a threshold being below and/or above a given threshold for a certain variable. The user interaction criteria can include one or more of a frequency of a low battery level being above a threshold battery level frequency, a misuse of a meal announcement control interface, a misuse of a cartridge replacement interface, a misuse of an infusion set replacement interface, a misuse of a glucose sensor replacement interface, a misuse of a therapy change control interface, a misuse of a pump settings interface, a frequency of device malfunction status alerts above an ordinary alert frequency, and/or other user interaction criteria. For example, the user interaction criteria may include a frequency of an occlusion state within a pump component above a threshold occlusion frequency. The occlusion state interferes with medicament delivery to the subject by the ambulatory medicament pump. The user interaction criteria can include a frequency and/or a duration of medicament level being below a medicament level threshold and/or a frequency of a glucose level signal being available below a threshold availability frequency. Other criteria are possible and these are listed by way of illustration only.

The misuse of the meal announcement control interface can include one or more of a plurality of misuses of the meal announcement control interface. For example, the misuse may include a number of mistimed uses of the meal announcement control interface, an amount of a meal announcement above a maximum threshold, an amount of a meal announcement below a minimum threshold, a number of a meal announcements above a maximum threshold, a number of a meal announcements below a minimum threshold, and/or a failure of use of the meal announcement control interface over a period of time. The ambulatory medicament pump and/or the control system may include the meal announcement interface. The meal announcement interface may be a graphical user interface that includes one or more features of other user interfaces described herein.

In some embodiments, a user may be able to select the training content and/or a time/duration associated with the training content. For example, the control system can receive (e.g., via user interaction with the meal announcement control interface) a request to access training content. The control system can transmit to a remote electronic device a request signal that is configured to request the additional training content. The transmission of the request signal may be in response to receiving the request to access additional training content.

in response to receiving the request to access additional training content, transmit, via the data interface to the remote electronic device, a request signal configured to request the additional training content

At block 2706 the control system can automatically generate the user alert comprising the link to the training content. The user alert may be generated in response to determining that the use state satisfies the at least one of the plurality of user interaction criteria. The link can direct a user to the training content in response to user interaction with the link. In some embodiments, the control system can transmit to a remote electronic device (e.g., remote server, a user device) an indication that the use state satisfies the at least one of the plurality of user interaction criteria. This may allow a different user (e.g., caretaker, manufacturer, etc.) to determine that the use state is improper and may require intervention.

In some embodiments, the control system can receive a user interaction with the link to access the training content. Based on the user interaction with the link, the control system may transmit to a remote electronic device (e.g., via the data interface) a report of the user interaction with the training content. Additionally or alternatively, the control system may receive a confirmation signal that can indicate that the report was received by the remote electronic device. The control system may generate via a user interface (e.g., a meal announcement control interface) a success alert configured to indicate that the report was successfully received by the remote electronic device. In some embodiments, the success alert may further indicate that the report constitutes a satisfaction of a training. In response to the satisfaction of the training, the control system may be prevented from transmitting additional training content for a period of time (e.g., a day, a week, etc.) to a user interface. The additional training content may be related to the specific use state for which the original training content was generated. For example, the control system may, in some implementations, not be prevented from generating additional training content related to a second different use state relative to a first use state.

The report of the user interaction may include a determination of a level of success associated with the user interaction. For example, a proper completion of the training content may result in generation of a success alert. The control system can transmit a third-party alert to a remote electronic device based at least in part on the user's interaction with the training content. The third-party alert can include an indication that the interaction with the training content was successful. Additionally or alternatively, the indication may include an attribute of the interaction with the training content, such as a score associated with a quiz or some other metric of success. Additionally or alternatively, a pausing of the training content may result in generation by the control system of an indication that the training content has not yet been completed. The system may identify that the training content has not been completed by a preset time. Based on this determination, the control system may generate a follow-up alert to the user to remind the user to complete the training content. The report of the user interaction may include results of the user interaction with test materials (e.g., test questions, analysis questions, fill-in-the blank, free form questions, etc.). The training content can include video (e.g., images, text, motion picture) and/or audio content.

In some embodiments, the control system may generate follow-up content. For example, in response to the user interaction with the link to access the training content, the control system may prompt the subject to participate in the follow-up content. The follow-up content may include at least one of a quiz, a survey, a setting of an alarm or reminder, and/or additional training content.

The control system may further transmit a request to receive the use state of the ambulatory medicament pump. This may allow the control system, which may be remote from the ambulatory medicament pump in some embodiments, to obtain the use state. Additionally or alternatively, the control system may receive (e.g., via the data interface from the remote electronic device) a confirmation signal that can indicate that the use state was received by the remote electronic device. Additionally or alternatively, the control system can generate a success alert configured to indicate that the use state was successfully received by the remote electronic device.

### Automated Support Contact

Medical devices such as ambulatory medicament devices can be complicated for users. Users may benefit from improved communication with support services that can ensure that the devices are properly used. It may be useful to have a control system (e.g., a glucose level control system), which may be part of an ambulatory medicament pump or other medicament device, that can transmit a request for a remote support system to contact a user and possibly identify a category of concern related to the request.

A system that can monitor the use of the ambulatory medicament device and reach out and/or contact a user when help is needed can promote proper use of the ambulatory medicament device and promote high quality care to the patient via the ambulatory medicament device. The system may be able to determine a use state of one or more of a plurality of pump components of an ambulatory medicament pump configured to be operatively connected to a subject. Based on the use state, the system can transmit a request for a remote support system to contact a user. The remote support system may be a help line, a support line, a manufacturer, a hospital, a caregiver (e.g., doctor, nurse, etc.), and/or some other person or other entity that can provide support to the user. This can aid a user in proper use of the medicament device, helping to avoid a lapse in therapy by helping ensure that the medicament device is properly attached, properly controlled, and/or properly used by a user (e.g., subject).

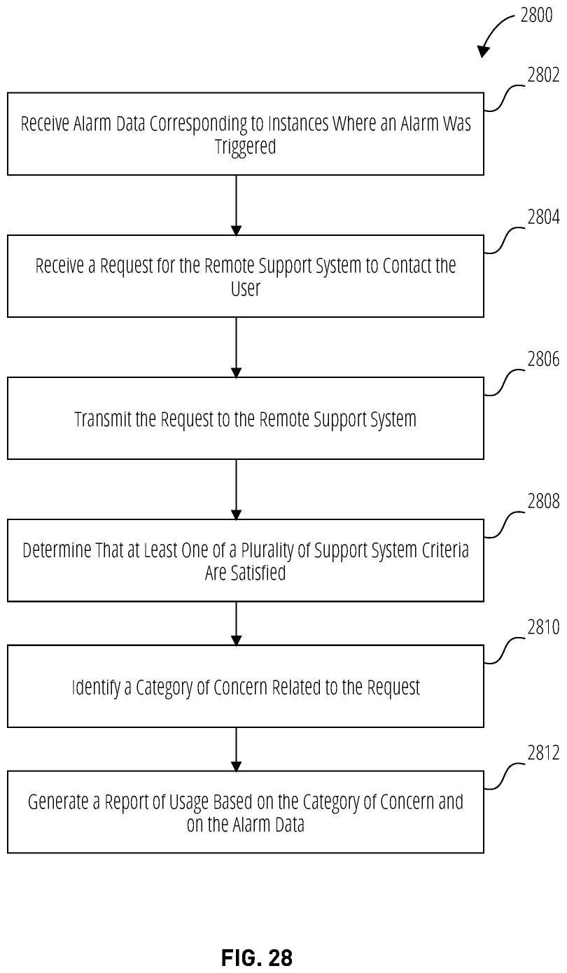

FIG. 28 shows a flow diagram illustrating an example method 2800 that may be used by a control system, such as one described herein (e.g., the glucose control system 308, the glucose control system 200b, the AMP 600, the glucose level control system 1102) to transmit a request for a remote support system to contact a user, such as via an ambulatory medical device (e.g., AMD 100, AMD 202, AMP 600, AMP 702, ambulatory medical device 500, etc.) and/or a related user interface.

At block 2802 the control system can receive (e.g., intermittently) from a pump monitoring system (e.g., one described herein) alarm data corresponding to instances where an alarm was triggered. The alarm may be one of the alarms described herein. At block 2804, the system can receive (e.g., via user interaction with a support request interface) a request for the remote support system to contact the user. At block 2806, the system can transmit, via the data interface, the request to the remote support system.

At block 2808, the system can determine that at least one of a plurality of support system criteria are satisfied, wherein the plurality of support system criteria include one or more criteria. Such support system criteria can include a determination that the alarm data comprise a number of alarms that exceeds a numerical threshold and/or a determination that the alarm data comprise a severity level of alarms that exceeds a severity threshold. The severity level can include a composite severity level of each alarm of the alarm data. Additionally or alternatively, the severity threshold can include at least one of a minimum threshold and/or a maximum threshold. The severity level may be based at least on a number of alarms and/or an elapsed time since generation of at least one of the alarms. The criteria can additionally or alternatively include a received request to contact the remote support system (e.g., a phone call, a transmitted text or audio message, an image, etc.). The received request to contact the remote support system can include an explanation for the requested contact with the remote support system. In some embodiments, the support system criteria may be provided by a manufacturer of the ambulatory medicament pump, a healthcare provider, the subject, an authorized user, or some other entity described herein. The control system may transmit a signal to the ambulatory medicament pump. The signal may be configured to reduce a rate of delivery of therapy to the subject and may be based on the determination that the at least one of the plurality of support system criteria are satisfied. Reducing the rate of delivery of therapy to the subject may include stopping the rate of delivery of therapy to the subject.

In some embodiments, the control system can determine that a threshold amount of time has passed since the determination that the alarm data comprises the severity level of alarms that exceeds the severity threshold. In response to the determination that the threshold amount of time has passed, the system can generate a follow-up alert, which may be configured to notify an individual that the threshold amount of time has passed, that the alarm data comprises the severity level of alarms that exceeds the severity threshold, and/or an inquiry into whether an action item associated with the at least one of a plurality of support system criteria has been addressed. The follow-up alert may be generated periodically, randomly, or according to a different alert timing.

At block 2810, the system can automatically identify a category of concern related to the request. The identification may be based on the use state of the at least one of the plurality of pump components of the ambulatory medicament pump.

At block 2812, the system can generate a report of usage based on the category of concern and on the alarm data. In some embodiments, the system can transmit the report of usage to a remote electronic device. Additionally or alternatively, the system can determine, based on the report of usage, a support resource that is configured to receive the report of usage associated with the glucose level control system. The system can receive a signal from the support resource. The signal can include one or more of a variety of signals. The signal can include an indication of a time when the user is expected to receive contact from the support resource. The signal can include an indication of a category of concern related to the request. The signal can include an indication of an identity of the support resource, an indication of the report of usage of the ambulatory medicament pump, and/or an indication of whether contact with the support resource is required, recommended, or optional. Additionally or alternatively, the signal can include an indication of a severity level associated with one or more alarms triggered by the glucose level control system.

In some embodiments, the control system can transmit (e.g., via the data interface) the report of usage to the support resource and/or generate a connection alert that is configured to request contact with a support request interface operatively coupled to the ambulatory medicament pump. The connection alert may be generated in response to user interaction with the connection alert. The control system can receive availability data associated with the remote support system and/or receive user selection of availability data associated with a user's availability. The system may generate the connection alert based at least on a number of different support system criteria that are satisfied, on a number of times that the at least one of the plurality of support system criteria are satisfied, on the category of concern, and/or on the report of usage. The connection alert may be generated periodically, randomly, or according to a different alert timing. For example, the connection alert may be repeated at variable time periods. The variable time periods may increase or decrease as time from an initial connection alert increases. For example, it may be beneficial to repeat a connection alert more frequently if a long time has passed since the initial connection alert was sent. Additionally or alternatively, a timing and/or nature of the connection alert may be based on a severity level of the alarms and/or on the time of the data. For example, the connection alert may be repeated more frequently during waking hours associated with the subject. Additionally or alternatively the system may transmit a signal to the ambulatory medicament pump that is configured to maintain rate of delivery of therapy to the subject at a minimum rate. The minimum rate may be configured to maintain a certain minimal level of medicament delivery while any issues surrounding the connection alert are addressed.

If the system determines that the appropriate support system criteria are satisfied, the system may transmit a signal to the ambulatory medicament pump that is configured to maintain rate of delivery of therapy to the subject at a normal rate. This may occur for example, if the support system criteria that are satisfied do not pose a health risk to the subject or are otherwise of a lower severity level.

Generating the connection alert can include contacting a manufacturer of the ambulatory medicament pump and/or a healthcare provider. Additionally or alternatively, generating the connection alert can include ordering one or more of medicament, a part for the ambulatory medicament pump, and/or an accessory for the ambulatory medicament pump, for example as described above.

The systems described herein can include a support system that is configured to receive a request to contact a user of a glucose level control system remote signal and to receive a report of usage comprising use data associated with the glucose level control system. The support system may be configured to receive the information that is sent by the control system and to send at least some of the data that is received by the control system. For example, the support system can receive, via a data interface, the remote signal and receive a report of usage that includes use data associated with the glucose level control system. The remote signal can include at least one of a request to contact the user of the glucose level control system (e.g., based on the report of usage) and/or status information associated with the glucose level control system. The support system may determine, based on the use data, a category of concern related to the request. The support system can transmit status information associated with the glucose level control system or availability data associated with the support system or to the glucose level control system. The support system may contact the user of the glucose level control system.

The support system can receive an indication that the at least one of the plurality of support system criteria (e.g., the support system criteria described above) associated with an ambulatory medicament pump are satisfied. The support system can receive user selection of availability data associated with a user's availability and/or receive user selection of availability data associated with a user's availability. Based on the category of concern, the support system may determine a target recipient of the report of usage and transmit the report of usage to the target recipient. The target recipient may be a department within an organization, such as a manufacturer help desk or support desk, a segment of a healthcare provider, or some other individual or group within an entity.

The support system may transmit (e.g., to the glucose level control system) data configured to generate a selector. The selector may be configured, in response to user selection of the selector, to allow a user to order supplies (e.g., via a user interface) for an ambulatory medicament pump. The support system may be configured to allow, based on user selection of the selector, a user to perform other actions remotely, as described herein.

### Distress Notifications

Delivering medication to a subject can require great care. Often the health of a subject may be volatile depending on the circumstances of the medicament needs of the subject, so there may be instances where a sudden or gradual circumstance arises that requires attention by the user. In such circumstances, it may be helpful for a medicament delivery system and/or an associated control system (e.g., a glucose level control system), which may be part of an ambulatory medicament pump or other medicament device, to track user behavior relative to a determination that a use state satisfies at least one of a plurality of user interaction criteria. Additionally or alternatively, the system may automatically generate a user alert that includes a link to training content.

A system that can monitor the use of the ambulatory medicament device and determine that certain information (“status information”) fails to satisfy one or more safety thresholds or satisfies one or more safety thresholds. Based on this determination, the system can automatically generate a distress alert. The status information can broadly include any device information pertaining to a condition of the ambulatory medicament device or subject information pertaining to a condition of a subject. Thus the status information can refer to any information that may be worthwhile in determining whether something about the system or how effective the medicament device is delivering medicament to the subject. Using the status information, the system can identify a problem or potential problem and take action to notify someone (e.g., the subject, a caretaker, another user, etc.) and/or modify therapy delivery, for example as described herein. This can help a user avoid a lapse in therapy by helping ensure that the pump parts are functioning properly and that they are delivering medicament properly.

In some embodiments, the system may receive sensing data from a sensing system which may include a geospatial location sensor, a motion sensor, a heart rate sensor, a temperature sensor, a skin-conductance sensor, and/or some other kind of physiological sensor. For example, the system may include a photoplethysmography (PPG) sensor to track heart-rate variability features such as a standard deviation of RR intervals and/or respiration rates. Such physiological responses can be triggered by changes in glucose level or physiological changes that may be related to glucose level (e.g., stress, anxiety, etc.). Additionally or alternatively, the sensing system may include an electrodermal activity (EDA) sensor, which can measure peak rise time, amplitude, and/or energy etc. These and/or other physiological measurements can help detect whether a person is in a normal or healthy state or whether the person is in need of urgent medical attention.

Additionally or alternatively, the sensing system may include a temperature sensor to track changes in a user's (e.g., patient's) skin temperature. Physiological or psychological stress may trigger peripheral vasoconstriction, causing a rapid, short-term drop in skin temperature.

Based on the sensing data from the sensing system, the system may determine that one or more safety thresholds are satisfied or are not satisfied and automatically generate a distress alert. The sensing data may broadly indicate a level of motion of the subject, an abnormal health condition of the subject, and/or another indicator relevant to other status information. The system may use the status information or the sensing data or a combination of both to identify a problem or potential problems and take action to ensure that the medicament is properly delivered to the subject.

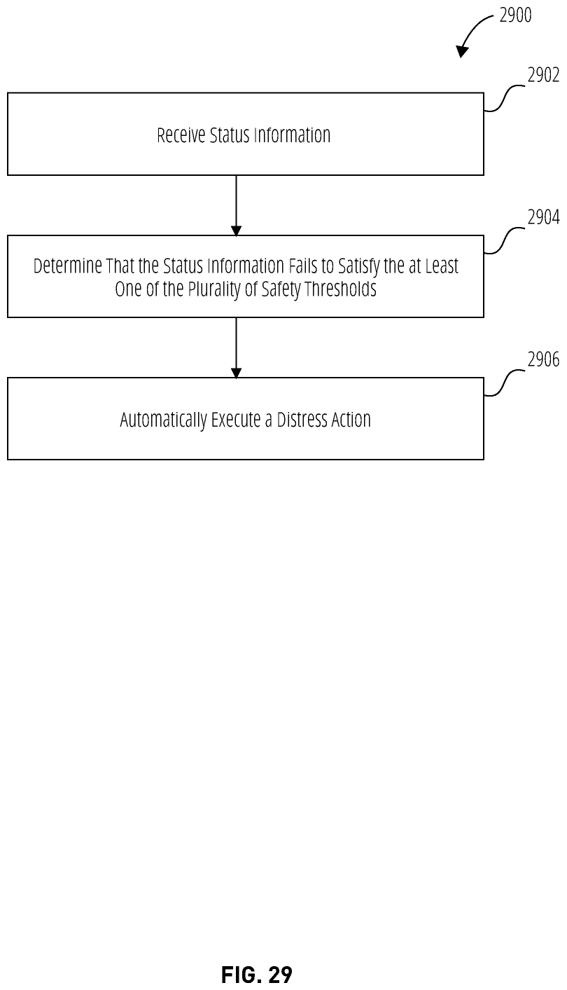

FIG. 29 shows a flow diagram illustrating an example method 2900 that may be used by a control system, such as one described herein (e.g., the glucose control system 308, the glucose control system 200b, the AMP 600, the glucose level control system 1102) to determine that status information fails to satisfy or satisfies at least one of a plurality of safety thresholds and to automatically generate a distress alert.

At block 2902 the control system can receive status information via a data interface. The receipt of the use state may be via the data interface. In some embodiments, the control system can receive sensing data from the sensing system via a sensing system interface. The sensing system interface may include software instructions for receiving and interpreting sensor system data. For example, in some embodiments the sensing system interface may be or consist of software instructions. At block 2904 the control system may determine that the status information fails to satisfy or satisfy the at least one of the plurality of safety thresholds. In some embodiments, this determination may be based at least in part on the sensing data received by the sensing system. The safety thresholds may be stored on a memory associated with the control system and/or may be received from a remote device (e.g., via the data interface). The safety thresholds can include one or more (e.g., a combination of) thresholds. Some examples of safety thresholds include a glucose level of a subject being less than a minimum threshold (e.g., indicative of a risk of hypoglycemia) and/or a glucose level of a subject being greater than a maximum threshold (e.g., a risk of hyperglycemia). The glucose level of the subject may be measured (e.g., by the glucose sensor) or predicted for a current or future time point or period of time.

Other thresholds may involve a movement of the subject as indicated by sensing data from a geospatial sensor, a motion sensor (e.g., accelerometer, gyroscope, magnetometer, etc.), and/or a physiological sensor. For example, a threshold may include movement that is less than a minimum movement threshold for a threshold amount of time (e.g., indicative of a subject that is possibly unconscious or otherwise unresponsive). The threshold may be configured to identify an unchanged position of the subject for at least a threshold amount of time. Additionally or alternatively, the threshold may include be configured to indicate a lack of motion of the subject for at least a threshold amount of time. This might indicate, for example, that a subject is not moving even while sleeping, which may be suggestive of a urgent problem that needs to be addressed. The threshold may take into account the time of day or other relevant factor. For example, a lack of motion from a geospatial sensor may generally trigger an alert during most times of the day but in some embodiments may not trigger an alert if the system determines that the subject is not expected to be in motion during that time (e.g., at night, during a nap, at a massage parlor, etc.).

The movement may be related to an abnormal or problematic health condition of the subject and indicate the subject's level of alertness (e.g., a subject that is unconscious, comatose, or lethargic). Such levels of alertness may be indicative of the subject being less responsive to stimuli and exhibit a decreased amount of responsiveness to their surroundings. The determination of movement may involve monitoring the subject's movement within a frame of reference (e.g., static frame of reference, constant velocity frame of reference, accelerating frame of reference) using sensing data received from a sensing system. The sensing system can be configured to hibernate or go into sleep mode during times of the day or periods when the subject is asleep and/or during periods of rest when the subject exhibits a negligible amount of movement. Distress alerts may be suppressed while the sensing system is in hibernation or sleep mode. The periods of hibernation may be manually set by a user and/or triggered by a negligible amount of movement or by a lack of activity of the subject as indicated by sensing data from motion sensors and/or sleep tracking devices (e.g., wearable devices, bedside sensors or bedside sensors). The sensing data can include body movement, breathing (e.g., breathing rate, etc.), heart data (e.g., heart rate, blood pressure, etc.), information about the room environment (e.g., temperature, humidity, ambient noise, light intensity, light color, etc.), and/or other data described herein. In some embodiments, for example, the system may use sound information (e.g., from a microphone) to determine an amount of movement of the user and/or a different condition of the user.

In some embodiments, the thresholds may include the movement of the subject being less than a minimum movement threshold of zero or a negligible amount of movement within a frame of reference for a threshold amount of time. The threshold amount of time may be between about 5 minutes and about 2 hours. Additionally, the minimum movement threshold may be combined with glucose level thresholds and/or amount of medicament thresholds to provide status information.

Other thresholds may related to an amount of medicament available, such as amount of medicament in a medicament cartridge being below a minimum threshold amount of medicament (e.g., indicative of a possible loss of critical therapy). Additionally or alternatively, a threshold can include a user input, such as a user selection of a distress notification (e.g., via a selection interface associate with the ambulatory medicament pump).

Other thresholds may relate to user (e.g., subject) behavior. For example, a threshold can involve a lack of data from a glucose level sensor (e.g., from improper use of the glucose level sensor, malfunction of the glucose level sensor, or lack of connectivity) for at least a threshold amount of time. Additionally or alternatively, the threshold can involve aberrant data from the glucose level sensor beyond an aberrant data threshold and/or aberrant usage of the glucose level control system beyond an aberrant usage threshold. Such aberrant data can include glucose level artifacts, which may not be associated with and/or reflective of a physiological glucose level of the subject. Such artifacts may be transient abnormal changes that can be identified based on characteristics of the corresponding glucose level signals (e.g., signal magnitude or temporal behavior of the signal magnitude). Some of the abnormal behaviors may include abrupt changes of magnitude or oscillations, discontinuous data trends, lack of smoothness, and/or glucose level readings that are not within an acceptable reference range. Such aberrant usage can include instances where there is a lack of interaction between the glucose level control system and the pump for a long period of time. In some embodiments, the threshold may include an alarm signal being active without being acknowledged for at least an alarm threshold period. For example, a problem may arise if the user does not acknowledge the alarm, thus potentially indicating that the user is in a state of emergency. A time that a user fails to acknowledge the alarm that exceeds the threshold period may trigger a distress action alone or in combination with other thresholds being exceeded, such as a low or empty cartridge, a low battery, an improper attachment and/or use of an ambulatory medicament pump, and/or some other threshold described herein. Low-level alarms may never trigger this threshold. In some embodiments, a user (e.g., the subject) may configure (e.g., select) one or more of the thresholds listed above. Additional features related to these thresholds are described in U.S. Patent Application Publication No. 2022/0080118, filed on Sep. 27, 2021, entitled “AMBULATORY MEDICAMENT DEVICE ALARM SYSTEM,” the entirety of which is hereby incorporated by reference herein for all purposes.

In some embodiments, a threshold can be related to malfunctioning of the system, including those thresholds already described above. For example, a threshold may include the ambulatory medicament device being in a non-functional state, such as when the device is not able to deliver medicament or otherwise unable to deliver therapy in the manner desired. Additionally or alternatively, a threshold can include a geolocation position of the glucose level control system being beyond a safety position threshold, such as when the data interface and/or the sensing system are located far from the glucose level control system or from a treatment and/or medicament delivery mechanism in communication with the glucose level control system. A safety position threshold may additionally or alternatively correspond to a position where a subject is expected or needed to be, such as at a particular time. Examples of such locations may be a treatment facility, a caregiver's location, a home of the subject, and/or some other location associated with the safety of the subject. The threshold may be based on a length of a time period (e.g., time away from the safety location) and/or on a time of the day/week. For example, a subject may be expected to be at the subject's own home during sleeping hours so a threshold distance away from the home for a particular amount of time may exceed the threshold. Other thresholds may be included as described herein.

The minimum threshold may be about 25 mg/dL, about 30 mg/dL, about 35 mg/dL, about 40 mg/dL, about 45 mg/dL, about 50 mg/dL, about 55 mg/dL, about 60 mg/dL, about 65 mg/dL, about 70 mg/dL, about 75 mg/dL, about 80 mg/dL, about 85 mg/dL, any glucose level therein, or fall within a range having endpoints thereof. For example, in some embodiments the minimum threshold is between 50 mg/dL and 70 mg/dL. The maximum threshold may be about 250 mg/dL, about 275 mg/dL, about 300 mg/dL, about 325 mg/dL, about 350 mg/dL, about 375 mg/dL, about 400 mg/dL, about 425 mg/dL, about 450 mg/dL, about 475 mg/dL, about 500 mg/dL, any glucose level therein, or fall within a range having endpoints thereof. For example, in some embodiments the maximum threshold is between 300 mg/dL and 400 mg/dL. The minimum movement threshold may be zero or a negligible amount of movement within a frame of reference. For example, even if the subject is moving within a vehicle, the system may determine that the subject has not moved within the frame of reference of the vehicle. The minimum threshold amount of medicament may be about 0 units, about 2 units, about 4 units, about 5 units, about 6 units, about 7 units, about 8 units, about 10 units, about 12 units, about units, any amount of units therein, or fall within a range having endpoints thereof. For example, in some embodiments the minimum threshold amount is between 0 units and 10 units of medicament.

The safety thresholds may be a combined set of thresholds. For example, the system may not take action unless a particular combination of thresholds is satisfied. For example, one combination may be the glucose level of a subject being less than the minimum threshold and the movement of the subject being less than the minimum movement threshold for the threshold amount of time. This combination may suggest that a user is unresponsive due to lack of medicament. Another combination may be the glucose level of a subject being greater than the maximum threshold and the movement of the subject being less than the minimum movement threshold for the threshold amount of time. Yet another combination may be the amount of medicament in the medicament cartridge being below the minimum threshold amount of medicament and the movement of the subject being less than the minimum movement threshold for the threshold amount of time. Other combinations are possible.

At block 2906 the system can automatically execute a distress action in response to determining that the status information fails to satisfy or satisfies the at least one of a plurality of safety thresholds. The distress action can include generating a distress alert, such as on a user interface described herein. Such distress alerts can include an audio call, a text message, and/or other data transmission, which can be sent to remote computing device, (e.g., to a caregiver, to an emergency response service, a family member). The distress alert can include at least one of the plurality of safety thresholds and/or a geolocation position (e.g., global positioning system (GPS), wireless network positioning, near-field location, Bluetooth Low Energy (BLE), Magnetic field detection, Near Field Communication (NFC), ultra-wideband, etc.) of the ambulatory medicament pump. For example, the distress alert may indicate what the safety threshold is that was exceeded. In some embodiments, the system can transmit the distress alert to a remote electronic device, such as one described herein. Additionally or alternatively, the distress alert can include a signal to suspend function of one or more elements of the ambulatory medicament pump or a signal to modify therapy delivered by the ambulatory medicament pump for a period of time. The therapy modification can include reducing an amount of insulin delivered by the ambulatory medicament pump. The period of time can be preset (e.g., by a user or a caregiver). This may help the medicament device avoid committing harmful actions, allow repair or analysis of the element, or some other benefit. Additionally or alternatively, the period of time may be automatically determined, and/or may be based at least in part on a duration or severity level associated with the failure to satisfy or satisfying the at least one of the plurality of safety thresholds.

In some embodiments, the system can determine that the status information no longer satisfies or fails to satisfy the at least one of the plurality of safety thresholds (e.g., after a matter of time). If the system determines this, it may generate a signal to resume the function of the one or more elements of the ambulatory medicament pump if they were suspended. Determining that the status information no longer satisfies or fails to satisfy the at least one of the plurality of safety thresholds can include receiving user interaction with the selection interface or with a selection interface of the ambulatory medicament pump. The user interaction with the selection interface can include the user overriding the distress alert and/or preventing the execution of further distress actions for a period of time, even if the status information still satisfies or fails to satisfy the at least one of the plurality of safety thresholds. For example, a user may be aware that an alert is improperly generated and so may choose to override the alert.

In some embodiments, a user may select (e.g., via a selection interface of a system described herein) an identity of a recipient of a distress alert, a time the recipient is to receive the distress alert, a condition under which the recipient is to receive the distress alert, a protocol for escalating delivery of the distress alert or subsequent alerts, and/or some other aspect of the alerts described herein or any of their associated thresholds.

Additionally or alternatively, the system (e.g., distress notification system, glucose level control system) may transmit a dose instruction (e.g., a dose control signal) to an associated ambulatory medicament pump.

The control system may receive (e.g., via the data interface from the remote electronic device) a confirmation signal that is configured to indicate that the distress alert was received by the remote electronic device and generate, via the selection interface, a success alert configured to indicate that the distress alert was successfully received by the remote electronic device.

In some embodiments, the system can transmit a request to receive the status information associated with the ambulatory medicament pump. Additionally or alternatively the system may transmit to the remote electronic device an indication that the status information no longer satisfies or fails to satisfy the at least one of the plurality of safety thresholds, which may signal a resumption of medicament delivery, etc. The system may transmit a third-party alert to the remote electronic device based on a duration or severity level associated with the failure to satisfy or satisfying the at least one of the plurality of safety thresholds. The third-party alert can include an indication of at least the duration or severity level associated with the failure to satisfy or satisfying the safety threshold(s).

### Terminology

It is to be understood that not necessarily all objects or advantages may be achieved in accordance with any particular embodiment described herein. Thus, for example, those skilled in the art will recognize that certain embodiments may be configured to operate in a manner that achieves or optimizes one advantage or group of advantages as taught herein without necessarily achieving other objects or advantages as may be taught or suggested herein.

All of the processes described herein may be embodied in, and fully automated via, software code modules executed by a computing system that includes one or more computers or processors. The code modules may be stored in any type of non-transitory computer-readable medium or other computer storage device. Some or all the methods may be embodied in specialized computer hardware. Further, the computing system may include, be implemented as part of, or communicate with an automated blood glucose system, an ambulatory medicament system, or an ambulatory medical device.

Many other variations than those described herein will be apparent from this disclosure. For example, depending on the embodiment, certain acts, events, or functions of any of the algorithms described herein can be performed in a different sequence, can be added, merged, or left out altogether (for example, not all described acts or events are necessary for the practice of the algorithms). Moreover, in certain embodiments, acts or events can be performed concurrently, for example, through multi-threaded processing, interrupt processing, or multiple processors or processor cores or on other parallel architectures, rather than sequentially. In addition, different tasks or processes can be performed by different machines and/or computing systems that can function together.

The various illustrative logical blocks and modules described in connection with the embodiments disclosed herein can be implemented or performed by a machine, such as a processing unit or processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable device that performs logic operations without processing computer-executable instructions. A processor can also be implemented as a combination of computing devices, for example, a combination of a DSP and a microprocessor, a plurality of microprocessors, one or more microprocessors in conjunction with a DSP core, or any other such configuration. Although described herein primarily with respect to digital technology, a processor may also include primarily analog components. A computing environment can include any type of computer system, including, but not limited to, a computer system based on a microprocessor, a mainframe computer, a digital signal processor, a portable computing device, a device controller, or a computational engine within an appliance, to name a few.

Conditional language such as, among others, “can,” “could,” “might” or “may,” unless specifically stated otherwise, are otherwise understood within the context as used in general to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Disjunctive language such as the phrase “at least one of X, Y, or Z,” unless specifically stated otherwise, is otherwise understood with the context as used in general to present that an item, term, etc., may be either X, Y, or Z, or any combination thereof (for example, X, Y, and/or Z). Thus, such disjunctive language is not generally intended to, and should not, imply that certain embodiments require at least one of X, at least one of Y, or at least one of Z to each be present.

Any process descriptions, elements or blocks in the flow diagrams described herein and/or depicted in the attached figures should be understood as potentially representing modules, segments, or portions of code which include one or more executable instructions for implementing specific logical functions or elements in the process. Alternate implementations are included within the scope of the embodiments described herein in which elements or functions may be deleted, executed out of order from that shown, or discussed, including substantially concurrently or in reverse order, depending on the functionality involved as would be understood by those skilled in the art.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

Many variations and modifications may be made to the above-described embodiments, the elements of which are to be understood as being among other acceptable examples. All such modifications and variations are intended to be included herein within the scope of this disclosure.

What is claimed is:

1. A glucose level control system configured to determine that status information satisfies at least one of a plurality of safety thresholds and to automatically execute a distress action, the glucose level control system comprising:
a data interface configured to connect to a remote electronic device and to an associated ambulatory medicament pump;
a sensing system comprising at least one of a geospatial location sensor or a motion sensor;
a non-transitory memory configured to store specific computer-executable instructions and the status information comprising at least one of device information pertaining to a condition of the ambulatory medicament device or subject information pertaining to a condition of a subject; and
a hardware processor in communication with the non-transitory memory and configured to execute the specific computer-executable instructions to at least:
receive the status information via the data interface;
receive sensing data from the sensing system, the sensing data indicating at least a level of motion of the subject;
determine that the status information satisfies the at least one of the plurality of safety thresholds, wherein the plurality of safety thresholds comprises:
a glucose level of a subject being less than a first threshold;
a glucose level of a subject being greater than a second threshold;
a lack of data from a glucose level sensor for at least a threshold amount of time;
the sensing data from the geospatial sensor indicating an unchanged position of the subject for at least a second threshold amount of time;
the sensing data from the motion sensor indicating a lack of motion of the subject for at least a third threshold amount of time;
aberrant data from the glucose level sensor beyond an aberrant data threshold;
aberrant usage of the glucose level control system beyond an aberrant usage threshold; and
the ambulatory medicament device being in a non-functional state; and
in response to determining that the status information satisfies the at least one of the plurality of safety thresholds and based at least in part on the sensing data, automatically execute a distress action and transmit, via the data interface, a dose instruction to the associated ambulatory medicament pump, wherein executing the distress action comprises transmitting a distress signal to a remote electronic device.

2. The glucose level control system of claim 1, wherein executing the distress action comprises generating a distress alert.

3. The glucose level control system of claim 2, wherein generating the distress alert comprises receiving a user input configured to indication of receipt of the distress alert.

4. The glucose level control system of claim 1, wherein executing the distress action comprises transmitting, via the data interface, an indication of the status information satisfying the at least one of the plurality of safety thresholds.

5. The glucose level control system of claim 2, wherein the distress alert comprises an indication of the at least one of the plurality of safety thresholds or of a geolocation position of the ambulatory medicament pump.

6. The glucose level control system of claim 2, wherein the electronic processor is further configured to execute the specific computer-executable instructions to at least:
transmit the distress alert to a remote electronic device.

7. The glucose level control system of claim 6, wherein transmitting the distress alert to a remote electronic device comprises initiating an audio call or transmitting a text message or other data to a remote computing device.

8. The glucose level control system of claim 6, wherein the electronic processor is further configured to execute the specific computer-executable instructions to at least:
receive, via the data interface from the remote electronic device, a confirmation signal configured to indicate that the distress alert was received by the remote electronic device; and
generate, via the selection interface and based on receiving the confirmation signal, a success alert configured to indicate that the distress alert was successfully received by the remote electronic device.

9. The glucose level control system of claim 1, wherein executing the distress action comprises generating a signal to suspend function of one or more elements of the ambulatory medicament pump for a period of time.

10. The glucose level control system of claim 1, wherein executing the distress action comprises generating a signal to modify therapy delivered by the ambulatory medicament pump.

11. The glucose level control system of claim 10, wherein modifying the therapy delivered by the ambulatory medicament pump comprises reducing an amount of insulin delivered by the ambulatory medicament pump.

12. The glucose level control system of claim 10, wherein the period of time comprises a preset period of time.

13. The glucose level control system of claim 10, wherein the period of time is based at least in part on a duration or severity level associated with the satisfying the at least one of the plurality of safety thresholds.

14. The glucose level control system of claim 10, wherein the electronic processor is further configured to execute the specific computer-executable instructions to at least:
determine that the status information no longer satisfies the at least one of the plurality of safety thresholds; and
in response to the determination that the status information no longer fails to satisfy the at least one of the plurality of safety thresholds, generate a signal to resume the function of the one or more elements of the ambulatory medicament pump.

15. The glucose level control system of claim 14, wherein determining that the status information no longer satisfies the at least one of the plurality of safety thresholds comprises receiving user interaction with the selection interface or with a selection interface of the ambulatory medicament pump.

16. The glucose level control system of claim 15, wherein receiving the user interaction with the selection interface comprises preventing execution of further distress actions for a period of time.

17. The glucose level control system of claim 1, wherein the electronic processor is further configured to execute the specific computer-executable instructions to at least:
transmit a request to receive the status information associated with the ambulatory medicament pump.

18. The glucose level control system of claim 1, wherein determining that the status information satisfies the at least one of the plurality of safety thresholds comprises determining that the status information satisfies two or more of the plurality of safety thresholds.

19. The glucose level control system of claim 18, wherein the two or more of the plurality of safety thresholds comprises:
the glucose level of a subject being less than the first threshold; and
the movement of the subject being less than the minimum movement threshold for the threshold amount of time.

20. The glucose level control system of claim 18, wherein the two or more of the plurality of safety thresholds comprises:
the glucose level of a subject being greater than the second threshold; and
the movement of the subject being less than the minimum movement threshold for the threshold amount of time.

21. The glucose level control system of claim 18, wherein the two or more of the plurality of safety thresholds comprises:
the amount of medicament in the medicament cartridge being below the minimum threshold amount of medicament; and
the movement of the subject being less than the minimum movement threshold for the threshold amount of time.

22. The glucose level control system of claim 1, further comprising the selection interface.

23. The glucose level control system of claim 1, wherein the electronic processor is further configured to execute the specific computer-executable instructions to at least:
transmit, via the data interface to the remote electronic device, an indication that the status information no longer satisfies the at least one of the plurality of safety thresholds.

24. The glucose level control system of claim 1, wherein the electronic processor is further configured to execute the specific computer-executable instructions to at least:
transmit a third-party alert to the remote electronic device based at least in part on a duration or severity level associated with the satisfying the at least one of the plurality of safety thresholds, the third-party alert comprising indication of at least the duration or severity level associated with the satisfying the at least one of the plurality of safety thresholds.

25. The glucose level control system of claim 1, wherein the data interface comprises a wireless wide area network modem.

26. The glucose level control system of claim 25, wherein the wireless wide area network modem comprises a long-term evolution (LTE) modem.

27. The glucose level control system of claim 1, further comprising a pump monitoring system configured to determine the device information pertaining to the condition of the ambulatory medicament device.

28. The glucose level control system of claim 1, wherein the first threshold is between 50 and 70 mg/dL.

29. The glucose level control system of claim 1, wherein the second threshold is between 300 and 400 mg/dL.

30. The glucose level control system of claim 1, wherein the threshold amount of time is between 5 minutes and 2 hours.

31. The glucose level control system of claim 1, wherein the minimum movement threshold is zero or a negligible amount of movement within a frame of reference.

32. The glucose level control system of claim 1, wherein the minimum threshold amount of medicament is between 0 and 10 units of medicament.

33. The glucose level control system of claim 1, wherein the glucose level of the subject comprises a measured glucose level or a predicted glucose level.

34. The glucose level control system of claim 1, wherein the electronic processor is further configured to execute the specific computer-executable instructions to at least:
receive, from a distress notification system, the plurality of safety thresholds.

35. An ambulatory medicament pump, comprising:
the glucose level control system of claim 1; and
a pump mechanism configured to deliver medicament to a subject.

36. A glucose level control system configured to determine that status information satisfies at least one of a plurality of safety thresholds and to automatically generate a distress alert, the glucose level control system comprising:
a data interface configured to connect to a remote electronic device and to an associated ambulatory medicament pump;
a non-transitory memory configured to store specific computer-executable instructions and status information comprising at least one of device information pertaining to a condition of the ambulatory medicament device or subject information pertaining to a condition of a subject; and
a hardware processor in communication with the non-transitory memory and configured to execute the specific computer-executable instructions to at least:
receive the status information via the data interface;
determine that the status information satisfies the at least one of the plurality of safety thresholds, wherein the plurality of safety thresholds comprises:
(i) a glucose level of a subject being less than or equal to a first threshold between 50 mg/dL and 70 mg/dL and (ii) a movement of the subject being equal to or less than a negligible amount of movement within a frame of reference for at least a threshold amount of time between 5 minutes and 1 hour;
(i) a glucose level of a subject being greater than or equal to a second threshold between 300 mg/dL and 400 mg/dL and (ii) a movement of the subject being equal to or less than the negligible amount of movement within the frame of reference for at least the threshold amount of time;
(i) an amount of medicament in a medicament cartridge being below a minimum threshold amount of medicament and (ii) a movement of the subject being less than a minimum movement threshold for the threshold amount of time; and
a user selection of a distress notification via a selection interface associate with the ambulatory medicament pump; and
in response to determining that the status information satisfies the at least one of a plurality of safety thresholds, automatically execute a distress action.

37. A distress notification system configured to determine that status information satisfies at least one of a plurality of safety thresholds and to automatically execute a distress action, the distress notification system comprising:
a data interface configured to connect to a glucose level control system;
a sensing system interface configured to receive sensing data that indicates at least a level of motion of the subject;
a non-transitory memory configured to store specific computer-executable instructions and the status information comprising at least one of device information pertaining to a condition of the ambulatory medicament device or subject information pertaining to a condition of a subject; and
a hardware processor in communication with the non-transitory memory and configured to execute the specific computer-executable instructions to at least:
receive the status information via the data interface;
receive, via the sensing system interface, the sensing data;
determine that the status information satisfies the at least one of the plurality of safety thresholds, wherein the plurality of safety thresholds comprises:
a glucose level of a subject being less than a first threshold;
a glucose level of a subject being greater than a second threshold;
a movement of the subject determined from sensing data being less than a minimum movement threshold for a threshold amount of time;
an amount of medicament in a medicament cartridge being below a minimum threshold amount of medicament;
a lack of data from a glucose level sensor for at least the threshold amount of time;
the sensing data from a geospatial sensor indicating an unchanged position of the subject for at least a second threshold amount of time;
the sensing data from a motion sensor indicating a lack of motion of the subject for at least a third threshold amount of time;
aberrant data from the glucose level sensor beyond an aberrant data threshold;
an alarm signal being active without being acknowledged for at least an alarm threshold period;
aberrant usage of the glucose level control system beyond an aberrant usage threshold;
the ambulatory medicament device being in a non-functional state;
a geolocation position of the glucose level control system being beyond a safety position threshold; and
a user selection of a distress notification via a selection interface associated with the ambulatory medicament pump; and
in response to determining that the status information satisfies the at least one of a plurality of safety thresholds and based at least in part on the sensing data, automatically execute a distress action, wherein executing the distress action comprises transmitting a distress signal to a remote electronic device.

38. The distress notification system of claim 37, wherein executing the distress action comprises at least one of generating a distress alert or transmitting a distress notification to a remote computing device.

39. The distress notification system of claim 37, further comprising the selection interface.

40. The distress notification system of claim 39, wherein the electronic processor is further configured to execute the specific computer-executable instructions to at least:
receive, via the selection interface, user selection of at least one of the first threshold, the second threshold, the minimum movement threshold, the threshold amount of time, the minimum threshold amount of medicament, the aberrant data threshold, the alarm threshold period, the aberrant usage threshold, or the safety position threshold.

41. The distress notification system of claim 39, wherein the electronic processor is further configured to execute the specific computer-executable instructions to at least:
receive, via the selection interface, user selection configured to dismiss or override the distress alert.

42. The distress notification system of claim 39, wherein the electronic processor is further configured to execute the specific computer-executable instructions to at least:
receive, via the selection interface, user selection configured to identify a recipient of the distress alert, a time the recipient is to receive the distress alert, a condition under which the recipient is to receive the distress alert, or a protocol for escalating delivery of the distress alert or subsequent alerts.

43. The glucose level control system of claim 1, wherein the sensing system comprises at least one of a heart rate sensor, a temperature sensor, or a skin conductance sensor.
